(19) United States
(12) Patent Application Publication
(10) Pub. No.: US 2024/0315962 A1
(43) Pub. Date: Sep. 26, 2024

(54) EXTENDED RELEASE COMPOSITIONS COMPRISING PYRIDOSTIGMINE

(71) Applicant: AMNEAL COMPLEX PRODUCTS RESEARCH LLC, Bridgewater, NJ (US)

(72) Inventors: Siva Ram Kiran Vaka, Piscataway, NJ (US); Namdev B. Shelke, Somerville, NJ (US); Dipen Desai, Basking Ridge, NJ (US); Wantanee Phuapradit, Lewes, DE (US); Navnit H. Shah, Monmouth Junction, NJ (US)

(73) Assignee: AMNEAL COMPLEX PRODUCTS RESEARCH LLC, Bridgewater, NJ (US)

(21) Appl. No.: 18/119,477

(22) PCT Filed: Dec. 10, 2021
(86) PCT No.: PCT/US2021/062845

§ 371 Date: Mar. 9, 2023

Related U.S. Application Data
(60) Provisional application No. 63/123,529, filed on Dec. 10, 2020.

(51) Int. Cl. A61K 9/00; A61K 9/28; A61K 31/4425

(52) U.S. Cl. CPC A61K 9/0065; A61K 9/2813; A61K 9/282; A61K 9/284; A61K 9/2853; A61K 9/2866; A61K 31/4425

(57) ABSTRACT

The present disclosure provides extended release pyridostigmine compositions for symptomatic treatment of myasthenia gravis; pretreatment for exposure to the chemical nerve agent Soman; and/or the treatment of orthostatic dizziness, lightheadedness, or the “feeling that you are about to black out” in adult patients with symptomatic neurogenic orthostatic hypotension caused by primary autonomic failure (Parkinson's disease, multiple system atrophy, and pure autonomic failure). The compositions of the disclosure provide reduced Fluctuation Index; reduced/blunted Cmax; higher Cmin, reduced Cmax:Cmin ratio, and reduced initial burst release of the drug, as compared to marketed pyridostigmine products. The dosage forms include matrix tablets, gastroretentive tablets, and pellets, the latter being suitable for dosing in capsules, tablets, and sachets, as well as for sprinkling on foodstuffs.

### 1. RELATED APPLICATIONS

This application claims priority to U.S. Provisional Patent Application No. 63/123,529, filed Dec. 10, 2020, the disclosure of which is hereby incorporated by reference in its entirety.

### 2. TECHNICAL FIELD

The present disclosure provides extended release pyridostigmine compositions suitable for once-daily administration. The compositions are administered as a single dosage unit/day (QD) to provide extended release of pyridostigmine or a pharmaceutically acceptable salt thereof for at least about 8 hours (e.g., up to 24 hours). The extended release pyridostigmine compositions of the disclosure include matrix tablets, gastroretentive tablets, and pellets, the latter being suitable for dosing in capsules, tablets, and sachets, and for sprinkling on foodstuffs. In certain embodiments, the gastroretentive compositions of the disclosure include an immediate release (IR) layer (containing pyridostigmine or a pharmaceutically acceptable salt thereof) and an extended release (ER) component. The compositions are suitable for once-a-day administration and provide reduced/blunted Cmax; higher Cmin; reduced Cmax:Cmin ratio, and reduced initial burst release of the drug, as compared to marketed pyridostigmine products, e.g., MESTINON® and MESTINON® TIMESPAN®.

### 3. BACKGROUND

Pyridostigmine bromide is an active cholinesterase inhibitor that does not cross the blood-brain barrier. It works by increasing levels of acetylcholine, a chemical released by motor neurons to activate muscles. It is commonly used in muscle tone recovery in myasthenia gravis (MG), postoperative functional bowel bloating, and urinary retention. It has also been approved for combat use by United States military personnel, i.e., pyridostigmine bromide has been approved by the U.S. Food and Drug Administration (FDA) to increase survival after exposure to Soman “nerve gas” poisoning. Pyridostigmine is also known for modestly but significantly improving orthostatic hypotension without worsening supine hypertension.

The time-to-maximum peak plasma concentration of oral pyridostigmine is 1-2 hours and its elimination half-life is about 3-5 hours. Pyridostigmine undergoes hydrolysis by the enzyme cholinesterase and is metabolized in the liver. It is excreted in the urine as a combination of unchanged drug and pyridostigmine metabolites. The bioavailability of pyridostigmine is reported to be about 10-20% (NDA #020414). Due to suboptimal pharmacokinetics of pyridostigmine, including a short duration of action, MG patients must take multiple tablets, occasionally multiple times a day. The patients experience “wearing off” of the drug and worsening of symptoms prior to the next dose, suffer from poor tolerability at higher dose levels, and experience difficulty adhering to the required frequent dosing regimen.

The FDA has approved Valeant Pharmaceutical's MESTINON® (pyridostigmine bromide injection, suspension, tablets, and extended release (ER) tablets) for the treatment of MG. The MESTINON® injection contains 5 mg/ml pyridostigmine bromide; MESTINON® suspension contains 60 mg/teaspoon pyridostigmine bromide; MESTINON® tablets contain 60 mg pyridostigmine bromide; and ER MESTINON® TIMESPAN® tablets contain 180 mg pyridostigmine bromide. The average daily dose of pyridostigmine is ten 60 mg tablets, ten teaspoons of suspension, or between one and three 180 mg ER tablets, spaced to provide maximum relief. The ER 180 mg tablets are administered, as 1-3 tablets, depending upon severity of the condition, once- or twice-daily with an interval of at least 6 hours between doses.

The currently approved ER pyridostigmine products provide an initial burst release/dose dumping, followed by extended release of the remaining dose of pyridostigmine bromide. The approved ER formulations release about 35-55% of pyridostigmine after one hour, about 65-85% after four hours, and about 85% after eight hours (in vitro dissolution). As approximately 40-50% of the drug can be released during first hour with the approved/marketed ER product, it has limited clinical utility. Presently marketed pyridostigmine products are plagued by a spike in concentration, or dose dumping, while attempting to maintain therapeutic plasma concentrations of the drug for extended periods of time. Initial burst release/dose dumping of the drug is associated with various side effects, e.g., nausea, vomiting, diarrhea, abdominal cramps, fasciculations, weakness, increased peristalsis, increased salivation, increased bronchial secretions, miosis, and diaphoresis. Such an initial spike in vivo, causing unwanted side effects, can be compared with in vitro release of at least about 50% of the pyridostigmine bromide within two hours of dissolution into a dissolution medium mimicking gastric fluid condition.

It is particularly desirable for MG patients to have a constant level of pyridostigmine to improve therapeutic outcome and quality of life, and to reduce side effects.

Orthostatic hypotension (OH) refers to fall in blood pressure on standing that can result in hypoperfusion of organs, including brain. Neurogenic orthostatic hypotension (nOH) refers to OH caused by impairment of autonomic nervous system characterized by failure to provide adequate autonomic postural responses, most prominently systemic vasoconstriction and compensatory increase in heart rate (HR) sufficient to maintain blood pressure.

Neurogenic orthostatic hypotension may result from decreased availability of norepinephrine, leading to inadequate vasoconstriction on standing. Such decrease in availability of norepinephrine can be due to inadequate norepinephrine release, inadequate synthesis of norepinephrine, blunted central nervous system (CNS) drive, and/or impaired function of postganglionic sympathetic neurons. Benefits of norepinephrine replacement in treating nOH has been postulated based on role of norepinephrine in vasoconstriction.

Standing results in pooling of ˜500-1,000 ml of blood in lower extremities of the body and in splanchnic circulation (the blood flow to the abdominal gastrointestinal organs including the stomach, liver, spleen, pancreas, small intestine, and large intestine.). This may result in decrease in venous return to heart, and/or reduction in cardiac output. In normal healthy individuals, the autonomic nervous system maintains standard blood pressure upon standing by triggering of venous and arterial baroreceptors, which results in sympathetic activation providing vasoconstriction and increased venous return, increased heart rate, and increased blood pressure. However, patients with autonomic nervous system failure do not release required amount of norepinephrine (NE) upon standing, which results in the following: no increase in sympathetic outflow; no decrease in vagal nerve activity, no increase in peripheral resistance, venous return to the heart, or cardiac output; and not limited fall in blood pressure. Such patients with autonomic nervous system failure may exhibit hypoperfusion of brain (cerebral hypoperfusion), and hypoperfusion of other organs/tissues, which can result in symptomatic nOH.

Symptoms and signs of OH can include postural lightheadedness/dizziness, and/or sensation of blacking out, with or without syncope. Additional less common symptoms of OH can include orthostatic cognitive dysfunction, mental dulling, generalized weakness, leg buckling, fatigue, visual blurring, headache, neck pain or discomfort (“coat hanger” configuration), platypnea, orthostatic dyspnea, chest pain, and a combination thereof.

Primary causes of non-neurogenic orthostatic hypotension (OH) can include volume depletion, dehydration, vasodilation, hypovolemia, cardiac insufficiency, impaired venous return, and a combination thereof. Sometimes OH can result from iatrogenic effect caused by use of vasodilators, antihypertensives, and tricyclic antidepressants.

Orthostatic hypotension can be neurogenic hypotension (nOH). Neurogenic orthostatic hypotension can result from failure to release required amount of norepinephrine upon standing, primary autonomic failure (central and peripheral synucleinopathies), parkinson's disease, multiple system atrophy (MTA), pure autonomic failure (PAF), autonomic neuropathy, or a combination thereof. Additionally, dopamine β-hydroxylase deficiency, diabetic and nondiabetic autonomic neuropathy can also cause nOH. In patients with multiple system atrophy and Parkinson's disease, nOH can occur early and may precede other manifestations of the disease.

Neurogenic orthostatic hypotension has substantial impact on patient's life. Some patients with nOH may severely limit their movement and activity due to high levels of anxiety resulting from previous falls. Some patients with nOH exhibit reduction in physical fitness, decreased capacity to engage in everyday life, loss of independence, depression, social isolation, and/or increased morbidity. In some patients, incidence of syncope or presyncope is higher during orthostatic challenge at elevated body temperature. Incidence and severity of orthostatic hypotension is high in patients with primary autonomic failure during periods of elevated temperature. In Parkinson's disease patients, neurogenic orthostatic hypotension increases risk of fall.

NORTHERA™ is approved by the FDA for the treatment of orthostatic dizziness, lightheadedness, or the “feeling that you are about to black out” in adult patients with symptomatic neurogenic orthostatic hypotension caused by primary autonomic failure (Parkinson's disease, multiple system atrophy, and pure autonomic failure), dopamine beta-hydroxylase deficiency, non-diabetic autonomic neuropathy). However, the label for NORTHERA™ includes black box warning for supine hypertension. The label explicitly mentions monitoring supine blood pressure prior to and during treatment and more frequent when increasing doses; and warns to discontinue NORTHERA™ if supine hypertension cannot be managed by elevation of the head of the bed. It is desirable to modestly but significantly improve orthostatic hypotension without worsening supine hypertension. It is particularly desirable to treat orthostatic dizziness, lightheadedness, or the “feeling that you are about to black out” in adult patients with symptomatic neurogenic orthostatic hypotension caused by primary autonomic failure, without worsening supine hypertension.

There remains a need for ER pyridostigmine compositions for symptomatic treatment of Myasthenia Gravis, while minimizing the side effects, reducing pill-burden, and increasing patient compliance. There remains a need for ER pyridostigmine compositions for pretreatment for exposure to the chemical nerve agent Soman, while reducing pill burden. There remains a need for ER pyridostigmine compositions for treating all forms of neurogenic and symptomatic orthostatic hypotension, and orthostatic intolerance in patients with any etiology including nervous system disorders, endocrine disorders, and cardiovascular diseases. There remains a need in the art for t for ER pyridostigmine compositions for treating orthostatic dizziness, lightheadedness, or the “feeling that you are about to black out” in adult patients with symptomatic neurogenic orthostatic hypotension caused by primary autonomic failure, without worsening supine hypertension.

The present disclosure provides ER pyridostigmine compositions that are designed to reduce Fluctuation Index, increase Cₘᵢₙ, reduce Cₘₐₓ:Cₘᵢₙ ratio, prolong and maintain therapeutic plasma concentration of pyridostigmine or a pharmaceutically acceptable salt thereof, and minimize side effects by controlling the initial burst release of the drug. The ER pyridostigmine compositions minimize lag time to drug release, provide extended release with reduced initial burst release, and maintain a stable therapeutic plasma concentration of the drug for extended periods of time. The ER pyridostigmine compositions of the disclosure are suitable for once-a-day administration and reduce side effects associated with burst release of the drug.

### 4. SUMMARY

The present disclosure provides a method of treating at least one symptom of neurogenic orthostatic hypotension comprising orally administering to a person in need thereof an extended release composition comprising pyridostigmine or a pharmaceutically acceptable salt thereof, wherein the composition provides extended release of pyridostigmine or a pharmaceutically acceptable salt for at least about 8 hours. In certain embodiments, the at least one symptom is retinal hypoperfusion, muscle hypoperfusion, lung hypoperfusion, cerebral hypoperfusion, myocardial hypoperfusion, nonspecific symptoms, or a combination thereof. In certain embodiments, the retinal hypoperfusion is impaired vision; the muscle hypoperfusion is neck pain, shoulder pain, or a combination thereof; the lung hypoperfusion is orthostatic dyspnea; the cerebral hypoperfusion is dizziness, light headedness, pre-syncope, syncope, difficulty concentrating, headache, cognition, or a combination thereof; the myocardial hypoperfusion is angina; and the nonspecific symptoms are generalized weakness, falls, leg buckling, lethargy, fatigue, nausea, or a combination thereof.

In certain embodiments, the disclosure provides a method for treating neurogenic orthostatic hypotension, the method comprising orally administering to a person in need thereof an extended release composition comprising pyridostigmine or a pharmaceutically acceptable salt thereof, wherein the composition provides extended release of pyridostigmine or a pharmaceutically acceptable salt thereof for at least about 8 hours.

In certain embodiments, the disclosure provides a method for treating neurogenic orthostatic hypotension in a person that did not respond to other treatments, the method comprising orally administering to the person an extended release composition comprising pyridostigmine or a pharmaceutically acceptable salt thereof, wherein the composition provides extended release of pyridostigmine or a pharmaceutically acceptable salt thereof for at least about 8 hours.

In certain embodiments, the disclosure provides a method for treating neurogenic orthostatic hypotension as a first line treatment in persons that discontinued other treatments because of supine hypertension, the method comprising orally administering to the person an extended release composition comprising pyridostigmine or a pharmaceutically acceptable salt thereof, wherein the composition provides extended release of pyridostigmine or a pharmaceutically acceptable salt thereof for at least about 8 hours.

In certain embodiments, the disclosure provides a method for treating at least one symptom of neurogenic orthostatic hypotension caused by primary autonomic failure beta-hydroxylase deficiency, diabetic and/or non-diabetic autonomic neuropathy, the method comprising orally administering to a person in need thereof an extended release composition comprising pyridostigmine or a pharmaceutically acceptable salt thereof, wherein the composition provides extended release of pyridostigmine or a pharmaceutically acceptable salt thereof for at least about 8 hours. In certain embodiments, the primary autonomic failure comprises autonomic failure associated with Parkinson's disease (PD), autonomic failure associated with multiple system atrophy, or pure autonomic failure. In certain embodiments, the at least one symptom comprises orthostatic dizziness, lightheadedness, feeling like you might black out, cognitive slowing, sleepiness, presyncope, and syncope, increased risk of falls, cognitive impairment, exercise intolerance, or a combination thereof.

In certain embodiments, the disclosure provides a method for treating neurogenic orthostatic hypotension without worsening supine hypertension, the method comprising orally administering to a person in need thereof an extended release composition comprising pyridostigmine or a pharmaceutically acceptable salt thereof, wherein the composition provides extended release of pyridostigmine or a pharmaceutically acceptable salt thereof for at least about 8 hours.

In certain embodiments, the composition is suitable for once-a-day administration.

In certain embodiments, the composition is a gastroretentive composition.

In certain embodiments, the composition comprises from about 50 mg to about 300 mg, from about 100 mg to about 250 mg, or from about 105 mg to about 205 mg of pyridostigmine or a pharmaceutically acceptable salt thereof.

In certain embodiments, the composition comprises an immediate release portion and an extended release portion. In certain embodiments, the extended release portion comprises a core and a permeable elastic membrane comprising an orifice and surrounding the core. In certain embodiments, the immediate release portion comprises an immediate release drug layer comprising pyridostigmine or a pharmaceutically acceptable salt thereof.

In certain embodiments, the core comprises pyridostigmine or a pharmaceutically acceptable salt thereof, an acid, a gas generating agent, a filler, a wicking agent, a swellable water-soluble hydrophilic polymer, or combinations thereof.

In certain embodiments, the permeable elastic membrane comprises a plasticizer and a copolymer based on ethyl acrylate, methyl methacrylate, and trimethylammonioethyl methacrylate chloride. In certain embodiments, the copolymer is present in an amount of from about 60 wt % to about 95 wt %, based on the total weight of the membrane.

In certain embodiments, the treatment comprises an increase in diastolic blood pressure by at least about 5 mmHg post standing. In certain embodiments, the treatment comprises an increase in systolic blood pressure by at least about 10 mmHg post standing.

In certain embodiments, the disclosure provides a method for treating neurogenic orthostatic hypotension, the method comprising orally administering to a person in need thereof an extended release composition comprising pyridostigmine or a pharmaceutically acceptable salt thereof and midodrine or a pharmaceutically acceptable salt thereof, wherein the composition provides extended release of pyridostigmine or a pharmaceutically acceptable salt thereof and midodrine or a pharmaceutically acceptable salt thereof for at least about 8 hours.

In certain embodiments, the disclosure provides a method for making a gastroretentive dosage form comprising a core and a permeable elastic membrane containing an orifice and surrounding the core, the method comprising: mixing pyridostigmine or a pharmaceutically acceptable salt thereof with a glidant(s) to obtain a drug intermediate blend; mixing the drug intermediate blend with an acid(s), a gas generating agent(s), a wicking agent(s), a filler(s), and a swellable water soluble polymer(s) into a final blend; compressing the final blend into a tablet core; coating the tablet core with a functional coat/permeable elastic membrane comprising at least one copolymer of ethyl acrylate, methyl methacrylate, and trimethylammonioethyl methacrylate chloride, and a plasticizer(s), to obtain a membrane coated tablet core, and drilling an orifice through the membrane/functional coat.

In certain embodiments, the disclosure provides a method for making a gastroretentive dosage form comprising an immediate release portion comprising an immediate release drug layer; and an extended release portion comprising a core coated with a permeable elastic membrane comprising an orifice, the method comprising: mixing pyridostigmine or a pharmaceutically acceptable salt thereof with at least one glidant to obtain a drug intermediate blend; mixing the drug intermediate blend with at least one excipient comprising an acid, a gas generating agent, a wicking agent, a filler, a swellable water soluble polymer, or a combination thereof, into a final blend; compressing the final blend into a tablet core; coating the tablet core with a permeable elastic membrane comprising at least one copolymer of ethyl acrylate, methyl methacrylate, and trimethylammonioethyl methacrylate chloride, and at least one plasticizer, to obtain a membrane coated tablet core; drilling an orifice through the membrane to a obtain membrane coated tablet core comprising an orifice in the membrane, and coating the membrane coated tablet core comprising an orifice in the membrane with an immediate release drug layer comprising pyridostigmine or a pharmaceutically acceptable salt thereof.

In certain embodiments, the disclosure provides an extended release gastroretentive dosage form comprising pyridostigmine or a pharmaceutically acceptable salt thereof, wherein the dosage form is dose proportional at 105 mg, 205 mg, 275 mg, and 340 mg strengths, based on PK parameters comprising Cmax.

In certain embodiments, the disclosure provides a gastroretentive dosage form comprising an immediate release portion and an extended release portion, wherein the immediate release portion comprises an immediate release drug layer comprising pyridostigmine or a pharmaceutically acceptable salt thereof, wherein the extended release portion comprises a core, and a permeable elastic membrane comprising at least one orifice and surrounding the core, wherein the core comprises a drug intermediate blend, and at least one excipient comprising an acid, a gas-generating agent, a wicking agent, a filler, a swellable water-soluble hydrophilic polymer, or a combination thereof; wherein the drug intermediate blend comprises pyridostigmine or a pharmaceutically acceptable salt thereof and at least one glidant; wherein the permeable elastic membrane comprises at least one copolymer of ethyl acrylate, methyl methacrylate, and trimethylammonioethyl methacrylate chloride and at least one plasticizer.

In certain embodiments, the disclosure provides a gastroretentive dosage form comprising a core, and a permeable elastic membrane comprising at least one orifice and surrounding the core, wherein the core comprises a drug intermediate blend, and at least one excipient comprising an acid, a gas-generating agent, a wicking agent, a filler, a swellable water-soluble hydrophilic polymer, or a combination thereof; wherein the drug intermediate blend comprises pyridostigmine or a pharmaceutically acceptable salt thereof and at least one glidant; wherein the permeable elastic membrane comprises at least one copolymer of ethyl acrylate, methyl methacrylate, and trimethylammonioethyl methacrylate chloride and at least one plasticizer.

In certain embodiments, the acid is an organic acid. In certain embodiments, the organic acid is selected from the group consisting of succinic acid, citric acid, acetic acid, malic acid, fumaric acid, stearic acid, tartaric acid, boric acid, benzoic acid, and a combination thereof.

In certain embodiments, the gas generating agent is selected from the group consisting of carbonate and bicarbonate salts of alkali and alkaline earth metals. In certain embodiments, the gas generating agent is selected from the group consisting of sodium bicarbonate, sodium carbonate, magnesium carbonate, and calcium carbonate.

In certain embodiments, the wicking agent is crospovidone.

In certain embodiments, the filler is selected from the group consisting of lactose monohydrate, anhydrous lactose, directly compressible starches, hydrolyzed starches, pregelatinized starch, microcrystalline cellulose, silicified microcrystalline cellulose, carboxymethylcellulose and other cellulose polymers, sucrose and sucrose-based materials, dextrose, dibasic calcium phosphate anhydrous, dibasic calcium phosphate dihydrate, tricalcium phosphate, calcium sulfate dihydrate, and other alkaline inorganic salts, sugar alcohols such as mannitol, sorbitol, and xylitol, confectioner's sugar, and a combination thereof.

In certain embodiments, the swellable water-soluble hydrophilic polymer is hydroxypropyl methylcellulose. In certain embodiments, the hydroxypropyl methylcellulose is a mixture low viscosity hydroxypropyl methylcellulose and a high viscosity hydroxypropyl methylcellulose. In certain embodiments, the low viscosity hydroxypropyl methylcellulose has a viscosity of from about 50 mPa·s to about 2,400 mPa·s, and a weight average molecular weight of from about 150,000 Da to about 300,000 Da. In certain embodiments, the high viscosity hydroxypropyl methylcellulose has a viscosity of from about 2,500 mPa·s to about 300,000 mPa·s, and a weight average molecular weight of from about 350,000 Da and about 1,500,000 Da. In certain embodiments, the low viscosity hydroxypropyl methylcellulose and the high viscosity hydroxypropyl methylcellulose are present in equal wt %, based on the total weight of the core.

### 5. BRIEF DESCRIPTION OF THE DRAWINGS

FIGS. 1A and 1B depict schematic representations of pyridostigmine pellets, with and without an immediate release drug layer. FIG. 1A depicts a schematic representation of a pyridostigmine pellet containing a cellet core, an extended release drug layer, a seal coat and a functional coat. FIG. 1B depicts a schematic representation of a pyridostigmine pellet containing a cellet core, an extended release drug layer, a seal coat, a functional coat, a second seal coat, an immediate release drug layer, and an over coat.

FIGS. 2A and 2B depict schematic representations of pyridostigmine matrix tablets. FIG. 2A depicts a schematic representation of a pyridostigmine matrix tablets containing a matrix core containing pyridostigmine bromide, a functional coat and an over coat. FIG. 2B depicts a schematic representation of a pyridostigmine matrix tablets containing a matrix core containing pyridostigmine bromide, a functional coat, an immediate release drug layer containing pyridostigmine bromide, and an over coat.

FIGS. 3A and 3B depict schematic representations of pyridostigmine gastroretentive tablets. FIG. 3A depicts a schematic representation of a pyridostigmine gastroretentive tablet containing a core containing pyridostigmine bromide, a seal coat, a functional coat and an over coat. FIG. 3B depicts a schematic representation of a pyridostigmine gastroretentive tablet containing a core containing pyridostigmine bromide, a seal coat, a functional coat, an immediate release drug layer containing pyridostigmine bromide, and an over coat.

FIG. 4 compares dissolution profiles of pyridostigmine bromide from Tablets 8, 9, and 10, in about 900 ml of 50 mM pH 4.5 acetate buffer, using USP Apparatus I-Custom basket, at about 100 rpm and about 37° C.

FIG. 5 compares dissolution profiles of pyridostigmine bromide from Pellets 2 and 3, in 200 ml of 50 mM phosphate buffer at pH 6.8, using USP Apparatus II (Paddle), at about 50 rpm and about 37° C.

FIG. 6 compares dissolution profiles of pyridostigmine bromide from Pellets 9, 10, and 11, in 200 ml of 50 mM phosphate buffer at about pH 6.8, using USP Apparatus II (Paddle), at about 50 rpm and about 37° C.

FIG. 7 compares dissolution profiles of Tablets 8, 13, and 14 in about 900 ml of 50 mM pH 5.0 acetate buffer containing 150 mM NaCl, using USP Apparatus I (Custom Basket), at about 100 rpm and about 37° C. FIG. 7 shows that Tablets 13 and 14 (containing hydroxypropyl methylcellulose in an amount of about 30% w/w of the tablet core; and equimolar amounts of succinic acid and each of the two gas-generating agents) exhibit about 10-15% slower drug release compared to Tablet 8 (containing hydroxypropyl methylcellulose in an amount of about 20% w/w of the tablet core; and non-equimolar amounts of succinic acid and each of the two gas-generating agents).

FIG. 8 compares dissolution profiles of Tablets 13 and 14, each with and without a hole in the membrane/functional coat, and Tablet 8 (with a hole). The dissolution testing was conducted in about 250 ml of 0.001 N HCl containing 100 mM NaCl, using USP Apparatus III (BIO-DIS), at about 25 dpm and about 37° C.

FIG. 9 compares dissolution profiles of Tablet 8 (with a hole; “8-H”), and Tablets 14 and 14A, each with a hole (“H”) and without a hole in the membrane. The dissolution testing was conducted in about 900 ml of 50 mM pH 5.0 acetate buffer containing 150 mM NaCl, using USP Apparatus I (Custom Basket), at about 100 rpm and about 37° C.

FIG. 10 compares floating lag times of Tablets 8, 11, 13, and 15, with (“H”) and without a hole, at 200 mg functional coating weight gain, and Tablets 8A, 11A, 13A, and 15A, with and without a hole, at 250 mg functional coating weight gain. The flotation studies were performed using rotating bottle method at about 5 rpm and about 37° C., in 200 ml of 50 mM pH 4.5 acetate buffer containing 100 mM NaCl.

FIG. 11 compares volumetric expansion at flotation of Tablets 8, 11, 13, and 15, with (“H”) and without a hole, at 200 mg functional coating weight gain, and Tablets 8A, 11A, 13A, and 15A, with and without a hole, at 250 mg functional coating weight gain. FIG. 11 demonstrates that tablets without a hole exhibit higher volume expansion compared to tablets with a hole at flotation. The volume expansion studies were performed, using rotating bottle method at about 5 rpm and about 37° C., in 200 ml of 50 mM pH 4.5 buffer containing about 100 mM NaCl.

FIG. 12 compares volumetric expansion, at 90 minutes of Tablets 8, 11, 13, and 15, with (“H”) and without a hole, at 200 mg functional coating weight gain, and volumetric expansion, at one hour, of Tablets 8A, 11A, 13A, and 15A, with and without a hole, at 250 mg functional coating weight gain. FIG. 12 demonstrates that tablets without a hole exhibit higher volume expansion compared to tablets with a hole at both 90 minutes and one hour. The volume expansion studies were performed, using rotating bottle method at about 5 rpm and about 37° C., in 200 ml of 50 mM pH 4.5 acetate buffer containing 100 mM NaCl.

FIG. 13 compares volumetric expansion, and weight gain at 24 hours, of Tablets 8, 11, 13, and 15, with (“H”) and without a hole, at 200 mg functional coating weight gain. The volume expansion studies were performed, using rotating bottle method at about 5 rpm and about 37° C., in 200 ml of 50 mM pH 4.5 acetate buffer containing 100 mM NaCl. FIG. 13 demonstrates that tablets containing 200 mg of crospovidone (e.g., Tablets 11/11-H and 15/15-H) exhibit higher weight upon drying compared with tablets containing 100 mg of crospovidone (e.g., Tablets 8/8-H and 13/13-H).

FIG. 14 compares dissolution profiles of Tablets 8B, 15, 16, and 17 without a hole, and Tablets 8, 8B, 15, 16, and 17 with a hole (“H”), using BIO-DIS method at about 20 dpm and about 37° C., in 250 ml of 0.001 N HCl containing 100 mM NaCl. FIG. 14 demonstrates that tablets without a hole exhibit slower drug release rates compared to tablets with a hole.

FIG. 15 shows the effect of crospovidone on release rates of pyridostigmine from the gastroretentive compositions of the disclosure. FIG. 15 compares dissolution profiles of Tablets 8, 18, and 19 in about 900 ml of 50 mM pH 5.0 acetate buffer containing 150 mM NaCl, using USP Apparatus I (Custom Basket), at about 100 rpm and about 37° C. FIG. 15 demonstrates that tablets containing 200 mg of crospovidone (Tablets 18 and 19) exhibit faster drug release compared to a tablet containing 100 mg of crospovidone (Tablet 8).

FIG. 16 compares dissolution profiles of tablets containing a mixture of BENECEL™ K4M PH DC and METHOCEL™ K100 Premium DC (Tablets 20 and 21) and a tablet containing BENECEL™ K4M PH DC only (Tablet 8) in about 900 ml of 50 mM pH 5.0 acetate buffer containing 150 mM NaCl, using USP Apparatus I (Custom Basket), at about 100 rpm and about 37° C. FIG. 16 demonstrates that tablets containing the mixture (Tablets 20 and 21) provide more controlled release compared to a tablet containing BENECEL™ K4M PH DC only (Tablet 8).

FIG. 17 compares dissolution profiles of tablets (all with a hole (“H”)) containing an immediate release drug layer (Tablet 23) and tablets with no immediate release drug layer (Tablets 8 and 22) in about 900 ml of 50 mM pH 5.0 acetate buffer containing 150 mM NaCl, using USP Apparatus I (Custom Basket), at about 100 rpm and about 37° C. FIG. 17 demonstrates that the tablet containing an immediate release drug layer (Tablet 23) eliminates lag time compared to those that do not contain an immediate release drug layer (Tablets 8 and 22).

FIG. 18 compares pharmacokinetic data for gastroretentive Tablet 8 (T₁), pellet composition (T₂), and marketed pyridostigmine products, e.g., MESTINON® tablets (R₂) and ER MESTINON® (i.e., TIMESPAN®) tablets (R₁).

FIG. 19 provides schematic and photographic representations of the gastroretentive dosage form of the disclosure from its initial tablet form to its residue after drug release.

FIG. 20 compares pharmacokinetic data for gastroretentive Tablet 34, with a hole in the functional coat, under low fat-low calorie (LF-LC) breakfast conditions (Condition I) and high fat-high calorie (HF-HC) breakfast conditions (Condition II). FIG. 20 demonstrates that Tablet 34 provides a therapeutic plasma concentration of pyridostigmine for at least about 22 hours.

FIG. 21 compares pharmacokinetic data for gastroretentive Tablet 35, without a hole, under LF-LC breakfast conditions (Condition I) and HF-HC breakfast conditions (Condition II).

FIG. 21 demonstrates that Tablet 35 provides a therapeutic plasma concentration of pyridostigmine for at least about 22 hours.

FIG. 22 provides a steady state plasma concentration of pyridostigmine bromide from Tablet 34, day 5, based on a steady state simulation for Tablet 34 over a 5-day period. FIG. 22 demonstrates that Tablet 34 can provide and maintain therapeutic plasma concentrations of pyridostigmine, e.g., about 20 ng/ml, for a period of at least about 14 hours.

FIG. 23 compares in vitro dissolution profiles of a tablet containing an immediate release drug layer (Tablet 34), tablet with no immediate release drug layer (Tablet 8), and MESTINON® TIMESPAN®, in 900 ml of 50 mM pH 4.5 acetate buffer containing 100 mM NaCl, using USP Apparatus I (Custom Basket), at about 100 rpm and about 37° C. FIG. 23 demonstrates that Tablet 34 exhibits a substantial decrease in (e.g., elimination of) lag time compared to Tablet 8. FIG. 23 further demonstrates that Tablet 8 (without IR drug layer) exhibits minimized initial burst release; and Tablet 34 (with IR drug layer) provides an immediate release of a therapeutic amount of pyridostigmine bromide, with reduced initial burst release (less than about 35% drug release in about 2 hours) of the drug, compared to MESTINON® TIMESPAN®.

FIG. 24 compares pharmacokinetic data for gastroretentive Tablet 34, with a hole in the functional coat, under LF-LC breakfast conditions (Condition I) and HF-HC breakfast conditions (Condition II), and MESTINON® TIMESPAN®, under HF-HC breakfast conditions (Condition II). FIG. 24 demonstrates that MESTINON® TIMESPAN® provides higher drug plasma concentrations between about 0 and 5 hours compared to Tablet 34 under Conditions I and II. FIG. 24 further demonstrates that Tablet 34, under Conditions I and II, provides higher drug plasma concentrations over an extended time period, e.g., about 7 hours or beyond, compared to MESTINON® TIMESPAN®.

FIG. 25A provides volume expansion of Tablet 34 using rotating bottle method, at 5 rpm and 37° C., in 200 ml of 0.001 N HCL containing 10 mM NaCl. FIG. 25A demonstrates that the tablet exhibits 100% volume expansion at about 30 minutes, 200% volume expansion at about 1 hour, and 300% volume expansion at about 8 hours post-administration of the tablet into the dissolution medium.

FIG. 25B provides texture/compressibility force for Tablet 34 at various time points and corresponding volume expansions (see FIG. 25A), using TA.XT^{Plus} apparatus. FIG. 25B demonstrates that the compression force required to squeeze out the matrix core at 2 hours post-administration, at about 200% volume gain, was 30 N; at 8 hours post-administration, at about 300% volume gain, was 18.3 N; and at 24 hours post-administration, at about 250% volume gain, was 4.1 N.

FIG. 26 compares pharmacokinetic data, under fed conditions (MF-MC and HF-HC conditions), for gastroretentive Tablet 37, administered once-a-day, and marketed pyridostigmine bromide tablet, MESTINON® (60 mg), administered thrice-a-day.). FIG. 26 demonstrates that Tablet 37 provides a therapeutic plasma concentration of pyridostigmine for at least about 22 hours under MF-MC conditions and under HF-HC conditions. The data further indicates that the pyridostigmine composition of the disclosure (Tablet 37) exhibits minimal variability in PK profile, specially under HF-HC conditions, as compared to the marketed MESTINON® product (60 mg×3).

FIG. 27 compares dissolution profiles of Tablet 38, using USP Apparatus I, at 100 rpm and 37° C., in 900 ml of 0.1N HCl, 0.1N HCl containing 5% dissolved alcohol, 0.1N HCl containing 20% dissolved alcohol, and 0.1N HCl containing 40% dissolved alcohol.

FIG. 28 compares dissolution profiles of Tablet 38, using USP Apparatus I, at 100 rpm and 37° C., in 900 ml of 50 mM pH 5 buffer, pH 5 buffer containing 5% dissolved alcohol, pH 5 buffer containing 20% dissolved alcohol, and pH 5 buffer containing 40% dissolved alcohol.

FIG. 29 compares dissolution profiles of Tablet 39, using USP Apparatus I, at 100 rpm and 37° C., in 900 ml of 0.1N HCl, 0.1N HCl containing 5% dissolved alcohol, 0.1N HCl containing 20% dissolved alcohol, and 0.1N HCl containing 40% dissolved alcohol.

FIG. 30 compares dissolution profiles of Tablet 39, using USP Apparatus I, at 100 rpm and 37° C., in 900 ml of 50 mM pH 5 buffer, pH 5 buffer containing 5% dissolved alcohol, pH 5 buffer containing 20% dissolved alcohol, and pH 5 buffer containing 40% dissolved alcohol.

FIG. 31 provides a steady state plasma concentration, under fed conditions, of pyridostigmine bromide from Tablet 37 and MESTINON (60 mg×3), day 5-day 7, based on a steady state simulation over a 24 hour period.

FIG. 32 provides pharmacokinetic data for an open-label, non-randomized, four dose levels, four period, single ascending dose (SAD) study, conducted in 14 healthy, adult human subjects under fed (high fat and caloric content was defined as each meal containing (800-900 Kcal with ˜fat 50%) conditions, to evaluate dose proportionality of the proposed pyridostigmine bromide tablets, 105 mg, 205 mg, 275 mg and 340 mg.

FIG. 33 provides pharmacokinetic data for an open label, non-randomized, four treatment, four cohort, single period, sequential multiple ascending dose study to characterize the pharmacokinetics, safety and tolerability of pyridostigmine after once-daily administration of pyridostigmine bromide Tablet 38 (105 mg), Tablet 39 (205 mg), and Tablet 40 (275 mg), for six consecutive days in healthy, adult, human subjects under fed conditions [high fat and caloric content was defined as each meal containing (800-900 Kcal with ˜fat 50%)].

### 6. DETAILED DESCRIPTION

The presently disclosed subject matter provides extended release pyridostigmine compositions suitable for once-daily administration. In certain embodiments, the composition is suitable for twice-daily administration. In certain embodiments, the compositions of the disclosure provide dual-controlled release, e.g., membrane-controlled and matrix-controlled extended release, of pyridostigmine bromide. Such dual-controlled release results in maintaining therapeutic plasma concentration for extended periods, reduce Fluctuation Index, minimize dose dumping (blunted Cmax and reduced initial burst release) of pyridostigmine or a pharmaceutically acceptable salt thereof, e.g., pyridostigmine bromide, increase Cmin, and/or possibly overcome the gastrointestinal side effects associated with the currently marketed extended release pyridostigmine products. In certain embodiments, the lag time associated with the extended release compositions of the disclosure is eliminated/minimized with the presence of an immediate release drug layer comprising pyridostigmine or a pharmaceutically acceptable salt thereof. The extended release pyridostigmine compositions of the disclosure can be formulated as gastroretentive tablets, matrix tablets, and pellets suitable for dosing in capsules, tablets, sachets, and as sprinkled pellets on food. In certain embodiments, the pyridostigmine compositions can be formulated as gastroretentive tablets providing extended release of pyridostigmine or a pharmaceutically acceptable salt thereof. In certain embodiments, the compositions of the disclosure provide extended release of pyridostigmine or a pharmaceutically acceptable salt thereof, for at least about 8 hours, at least about 8 hours, at least about 9 hours, at least about 10 hours, at least about 11 hours, least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, or any intermediate periods therein. In certain embodiments, the disclosure provides methods for making matrix tablets, pellets, and gastroretentive tablets comprising pyridostigmine or a pharmaceutically acceptable salt thereof.

For clarity and not by way of limitation, this detailed description is divided into the following sections:
6.1. Definitions.
6.2. Pyridostigmine Dosage Forms.
6.3 Methods of Making; and
6.4 Methods of Treatment.

### 6.1. Definitions

The terminology used in the present disclosure is for the purpose of describing particular embodiments only and is not intended to be limiting. As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing,” and “comprising” are interchangeable, and one of skill in the art is cognizant that these terms are open-ended terms.

As used herein, “and/or” refers to and encompasses any and all possible combinations of one or more of the associated listed items. The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 15%, up to 10%, up to 5%, up to 1%, up to 0.5%, or even up to 0.1% of a given value. Unless otherwise defined, all terms, including technical and scientific terms used in the description, have the same meaning as commonly understood by one of ordinary skill in the art to which the present disclosure belongs. As used herein, “about” will be understood by persons of ordinary skill in the art and will vary to some extent on the context in which it is used. If there are uses of the term which are not clear to person of ordinary skill in the art given the context in which it is used, “about” will mean up to about ±10% of the particular term.

The terms, “a therapeutically effective amount” and “a therapeutically acceptable amount,” as used interchangeably herein, refer to an amount that will elicit a therapeutically useful response in a subject and includes an additional amount or overage of active ingredient deemed necessary in the formulation to provide the desired amount upon administration. The therapeutically useful response can provide some alleviation, mitigation, and/or decrease in at least one clinical symptom in the subject. Those skilled in the art will appreciate that the therapeutically useful response need not be complete or curative, as long as some benefit is provided to the subject. In some embodiments, the subject is a human. In certain embodiments, the therapeutically effective amount comprises pyridostigmine plasma level of from about 15 ng/ml to about 50 ng/ml.

The terms “treatment,” “treat,” and “treating,” as used interchangeably herein, refer to reversing, alleviating, delaying the onset of, and/or inhibiting the progress of a disease or disorder as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent or delay their recurrence.

As used herein, the term “immediate release” refers to release of at least 70% of a drug within one hour post-administration/consumption of the dosage form.

The terms “extended release” and “sustained release,” as used interchangeably herein, refer to dosage forms or compositions that are formulated to provide therapeutic drug concentrations over an extended period of time after administration, thereby allowing a reduction in dosing frequency, as compared to a drug presented as an immediate release dosage form.

As used herein, the term “floating” is used in conjunction with a “floating gastroretentive dosage form”, which has a bulk density less than gastric fluids. Such dosage forms are “floating” in that they remain buoyant in the gastric fluids of the stomach for a targeted period of time. The floating dosage form then is able to be retained in the stomach, while releasing an active agent.

The terms “floating lag time” and “lag time,” as used interchangeably herein, refer to the time between the addition of a dosage form to a medium and the time when the dosage form begins to float on the medium (e.g., in an in vitro setting), or the time between the consumption/administration of a dosage form by a user and the time when the dosage form begins to float on the surface of the gastric fluid (e.g., in an in vivo setting).

The terms “gastroretentive dosage form/drug delivery system, “gastroretentive oral floating dosage form/drug delivery system,” and “gastroretentive floating dosage forms/drug delivery systems,” as used interchangeably herein, refer to modified release dosage forms providing delayed gastric emptying as compared to food (e.g., retention in the stomach beyond the retention of food).

The term “pyridostigmine,” as used herein, refers to the pyridostigmine as well as all pharmaceutically acceptable salts, esters, and functionally equivalent chemical compounds of pyridostigmine.

The terms “initial burst release” and/or “dose dumping,” as used interchangeably herein, refer to an unintended initial spike in concentration of pyridostigmine or a pharmaceutically acceptable salt thereof, in extended release dosage forms.

The terms “reduced initial burst release,” and the like, as used herein, refer to in vitro release of less than about 35% of the pyridostigmine or a pharmaceutically acceptable salt thereof, within two hours of dissolution in 900 ml dissolution medium, measured using USP Apparatus I (Custom Basket), at about 100 rpm and about 37° C.

The terms “minimized initial burst release”, and the like, as used herein, refer to in vitro release of not more than 20% of the pyridostigmine or a pharmaceutically acceptable salt thereof, within two hours of dissolution in 900 ml dissolution medium, measured using USP Apparatus I (Custom Basket), at about 100 rpm and about 37° C.

The terms “Fluctuation Index” and “FI,” as used interchangeably herein with respect to the pyridostigmine dosage forms, refer to fluctuations in plasma levels of pyridostigmine released from the dosage form within a 24-hour dosing period. Fluctuation Index provides a quantitative measurement of fluctuation in drug plasma concentration, measured as dose related peak-trough fluctuations. Fluctuation Index is calculated using the formula: FI=(Cmax−Cmin)/Cav, wherein Cmax is the maximum plasma concentration of the drug, e.g., pyridostigmine; Cmin is the minimum plasma concentration of the drug; and Cav is the average plasma concentration of the drug.

The term “Cav,” as used herein with respect to the pyridostigmine dosage forms, for example MESTINON, MESTINON TIMESPAN®, and gastroretentive pyridostigmine dosage forms of the disclosure, refers to average plasma concentration of pyridostigmine or a pharmaceutically acceptable salt thereof, during a 24-hour dosing period.

The term “Cmax,” as used herein with respect to the pyridostigmine dosage forms, for example MESTINON, MESTINON TIMESPAN®, and gastroretentive pyridostigmine dosage forms of the disclosure, refers to maximum observed plasma concentration of active agent (e.g., pyridostigmine or a pharmaceutically acceptable salt thereof) or a metabolite, during a 24-hour dosing period, on a graph of the plasma concentration of the active agent vs. time. In certain embodiments, the term “Cmax” refers to maximum observed plasma concentration of active agent (e.g., pyridostigmine or a pharmaceutically acceptable salt thereof) or a metabolite, at steady state level, on a graph of the plasma concentration of the active agent vs. time.

The term “Cmin,” as used herein with respect to the pyridostigmine dosage forms, for example MESTINON®, MESTINON® TIMESPAN®, and gastroretentive pyridostigmine dosage forms of the disclosure, refers to minimum observed plasma concentration of active agent (e.g., pyridostigmine or a pharmaceutically acceptable salt thereof) or a metabolite, during a 24-hour dosing period, on a graph of the plasma concentration of the active agent vs. time. In certain embodiments, the term “Cmin” refers to minimum observed plasma concentration of active agent (e.g., pyridostigmine or a pharmaceutically acceptable salt thereof) or a metabolite, at steady state level, on a graph of the plasma concentration of the active agent vs. time.

The term “AUC_{0-∞},” as used herein, refers to total area under the concentration-time curve from time zero extrapolated to infinity. The area extrapolated to infinity (AUC_{t-∞}) is obtained by dividing the last quantifiable concentration by the terminal elimination rate Kₑₗ. AUC_{0-∞} is then determined by adding the extrapolated area AUC_{t-∞} to AUC₀₋ₜ. Kₑₗ is determined by unweighted linear least-squares regression analysis from the linear segment of the log concentration time data.

The term “AUC₀₋ₜ” as used herein, refers to area under the concentration-time curve from time zero to the last sample with quantifiable concentration, calculated using the linear trapezoidal method.

The term “tₘₐₓ,” as used herein, refers to time to peak concentration, taken to be the sampling time at which Cₘₐₓ was observed. Tmax refers to the time at which maximum plasma concentration of an active agent or a metabolite thereof is reached, during a 24-hour dosing period, on a graph of plasma concentration vs. time. In certain embodiments, Tmax refers to the time at which maximum plasma concentration of an active agent or a metabolite thereof is reached, at steady state level, on a graph of plasma concentration vs. time

The term “t_{1/2},” as used herein refers to terminal elimination half-life calculated by ln²/Kₑₗ.

The term “alcohol-induced dose dumping,” as used herein, refers to a rapid release of the entire dose or a significant fraction thereof in a short period of time.

The terms “pore former” and the like, as used herein, refer to water-soluble polymers and/or water-soluble small molecules that will form pores or channels (i.e., behave as a channeling agent) in the functional coat/membrane, thereby creating a permeable functional coat/membrane. The term “pore former” includes molecules used to create a certain amount of diffusion through an insoluble (or sparingly soluble) coating of a tablet, pellet, or particle to achieve an extended release profile.

The terms “gastric fluid,” and “GI fluid,” as used interchangeably herein, refer to medium occurring in stomach and/or lower GI (gastrointestinal) tract of an individual.

The terms “simulated gastric fluid,” and “SGF,” as used interchangeably herein, refer to a medium that is used to mimic the chemical environment of gastric fluid/medium in an in vitro setting.

As used herein, the term “dissolution medium” refers to a medium used to mimic pH of gastric fluid/medium in stomach or lower gastrointestinal tract of an individual. In certain embodiments, the medium used to mimic chemical environment of stomach of an individual includes a medium with pH of less than about 5.5, e.g., about 1, about 1.25, about 1.5, about 1.75, about 2, about 2.25, about 2.5, about 2.75, about 3, about 3.25, about 3.5, about 3.75, about 4.0, about 4.25, about 4.5, about 4.75, about 5.0, about 5.5, or any intermediate values therein. In certain embodiments, the medium used to mimic chemical environment of lower GI tract of an individual includes a medium with pH of from about 5.5 to about 8, e.g., about 5.5, about 5.75, about 6.0, about 6.25, pH 6.5, about 6.75, about 7, about 7.25, about 7.5, or any intermediate values therein.

In certain embodiments, the term “dissolution medium,” as used herein, refers to a biorelevant medium mimicking gastric fluid conditions. In certain embodiments, the biorelevant dissolution medium mimicking chemical environment of stomach of an individual comprises a medium with pH of less than about 5.5 and containing from about 1 mM to about 200 mM of NaCl. In certain embodiments, the biorelevant dissolution medium mimicking chemical environment of lower GI tract of an individual comprises a medium with pH of from about 5.5 to about 8 and containing from about 1 mM to about 200 mM of NaCl. In certain embodiments, the biorelevant dissolution medium comprises 50 mM pH 4.5 acetate buffer; 50 mM pH 4.5 acetate buffer with 100 mM NaCl; pH 5.0 acetate buffer with 150 mM NaCl; 0.01 N HCl with 100 mM NaCl; 0.01 N HCl; or a mixture of 150 mM NaCl, 30 mM sodium acetate, and 17 mM acetic acid.

In certain embodiments, the term “custom basket,” as used herein for use in USP Apparatus I, refers to a 40 mesh basket with 24.5 mm internal diameter and basket hub with 24.5 mm internal diameter.

The terms “swellable,” “swelling,” and the like, as used herein with respect to a polymer, refer to a polymer capable of imbibing fluid and swelling when in contact with a fluid environment.

The terms “expanding,” “expansion,” and the like, as used herein with respect to a permeable elastic membrane, refer to stretching or distention of a membrane due to the membrane elasticity, and an outward pressure, e.g., gas pressure, on the membrane.

The term “permeable,” as used herein, refers to a membrane containing sparingly soluble polymers, or insoluble polymers, with or without a pore former, that will allow the drug, e.g., pyridostigmine or a pharmaceutically acceptable salt thereof, to pass through the membrane by diffusion. In certain embodiments, the permeable membrane allows excipients and fluids to pass through membrane by diffusion. As used herein, the terms functional coat and permeable membrane are used interchangeably.

The terms “wicking agent,” and “disintegrants,” as used interchangeably herein, refer to a material(s) with the ability to draw and spread water into the core of the dosage form, thereby increasing the contact surface area of the drug with the incoming aqueous fluid. Wicking agents carry water to surfaces inside the core of the tablet to create channels or a network of increased surface area.

The term “low viscosity hydroxypropyl methylcellulose/hypromellose,” as used herein, refers to hydroxypropyl methylcellulose/hypromellose with a viscosity of from about 50 mPa·s to about 2,400 mPa·s, and a weight average molecular weight of from about 150,000 Da to about 300,000 Da.

The term “high viscosity hydroxypropyl methylcellulose/hypromellose,” as used herein, refers to hydroxypropyl methylcellulose/hypromellose with a viscosity of from about 2,500 mPa·s to about 300,000 mPa·s, and a weight average molecular weight of from about 350,000 Da and about 1,500,000 Da.

The term “dual-controlled release,” as used herein, refers to drug release from a membrane-controlled matrix (also referred to as a membrane-controlled matrix core or membrane-controlled core). The term “dual-controlled release” includes drug release that is controlled by both the matrix and the membrane portions of the dosage form, e.g., matrix-controlled and membrane-controlled release of pyridostigmine or a pharmaceutically acceptable salt thereof.

The term “orthostatic hypotension/OH,” as used herein refers to a fall in blood pressure on standing that can result in hypoperfusion of organs, including brain. Orthostatic hypotension (OH) refers to a sustained reduction of systolic blood pressure (SBP) of at least 20 mmHg and/or diastolic blood pressure (DBP) of 10 mmHg, within 3 min of standing or head-up tilt to at least 60° on a tilt table.

The term “neurogenic orthostatic hypotension,” as used herein, refers to orthostatic hypotension caused by impairment of autonomic nervous system characterized by failure to provide adequate autonomic postural responses, most prominently systemic vasoconstriction, and compensatory increase in heart rate (HR) sufficient to maintain blood pressure.

In certain embodiments, the terms “Orthostatic Hypotension/OH,” and neurogenic Orthostatic Hypotension/nOH,” are used interchangeably herein.

The term “Myasthenia Gravis,” as used herein, refers to a chronic autoimmune disorder in which antibodies destroy the communication between nerves and muscles, resulting in weakness of the skeletal muscles. Myasthenia gravis (MG) affects the voluntary muscles of the body, especially muscles controlling eyes, mouth, throat, and limbs.

### 6.2. Pyridostigmine Dosage Forms

The disclosed subject matter provides for extended release compositions containing pyridostigmine. The presently disclosed subject matter also provides for formulating the extended release compositions containing pyridostigmine into various dosage forms, such as, e.g., matrix tablets, gastroretentive tablets, and pellets. In certain embodiments, the present disclosure provides for dosage forms that contain an IR layer containing pyridostigmine bromide to eliminate the lag time associated with the presence of an extended release component alone. In certain embodiments, the extended release dosage forms of the disclosure either (1) contain an IR drug layer containing pyridostigmine bromide or (2) are administered with an IR pyridostigmine dosage form, in order to eliminate the lag time.

In certain embodiments, the extended release dosage forms of the disclosure, with or without an immediate release layer, provide reduced initial burst release of pyridostigmine or pharmaceutically acceptable salts thereof, e.g., pyridostigmine bromide, compared to marketed extended release pyridostigmine products. In certain embodiments, the extended release dosage forms of the disclosure, due to reduced initial burst release comprising release of less than 35% of the pyridostigmine or a pharmaceutically acceptable salt thereof, during first hour of oral ingestion, provide therapeutic effect while avoiding GI side effects.

In certain embodiments, the extended release dosage forms of the disclosure, with or without an immediate release drug layer, are formulated to reduce fluctuations in plasma levels of pyridostigmine throughout the day (24-hour period), as compared to marketed pyridostigmine products, e.g., MESTION and MESTINON TIMESPAN®. In certain embodiments, the extended release dosage forms of the disclosure, with or without an immediate release drug layer, are formulated to reduce dose related peak-to-trough fluctuations (Fluctuation Index) in plasma levels of pyridostigmine. In certain embodiments, a decrease in Fluctuation Index indicates a decrease in drug plasma fluctuation between the peak-to-trough plasma levels (e.g., Cmax:Cmin ratio) of pyridostigmine; and an increase in Fluctuation Index indicates an increase in drug plasma fluctuation between the peak-to-trough plasma levels (e.g., Cmax:Cmin ratio) of pyridostigmine. A Fluctuation Index of zero indicates no fluctuations in drug plasma levels, e.g., as observed in intra venous infusion at steady state. In certain embodiments, the extended release pyridostigmine compositions of the disclosure, upon a single dose administration, provide a Fluctuation Index from about 0.1 to about 1. In certain embodiments, the extended release pyridostigmine compositions of the disclosure provide a Fluctuation Index (Cmax−Cmin/Cav) of about 0.1, about 0.2, about 0.3, about 0.4, about 0.5, about 0.6, about 0.7, about 0.8, about 0.9, about 1, or any intermediate values therein.

In certain embodiments, the extended release pyridostigmine compositions of the disclosure release less than 35% of pyridostigmine (also referred to herein as “reduced initial burst release”) during first hour of oral ingestion. In certain embodiments, the extended release compositions of the disclosure avoid or minimize unwanted GI side effects, generally experienced with the currently marketed pyridostigmine products, e.g., MESTINON® and MESTINON® TIMESPAN®, by releasing less than 35% of pyridostigmine (also referred to herein as “reduced initial burst release”) during first hour of oral ingestion, providing consistent blood levels of pyridostigmine in a therapeutic range, reducing Fluctuation Index, lowering Cmax, and increasing Cmin. The extended release dosage forms of the disclosure, with or without the IR drug layer, minimize GI side effects, and provide and maintain therapeutic plasma concentrations of pyridostigmine for a period of at least about 8 hours. In certain embodiments, therapeutic plasma concentration of pyridostigmine is from about 15 ng/ml to about 50 ng/ml. In certain embodiments, therapeutic plasma concentration of pyridostigmine is about 15 ng/ml, about 16 ng/ml, about 17 ng/ml, about 18 ng/ml, about 19 ng/ml, about 20 ng·ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, or any intermediate values therein.

In certain embodiments, the extended release pyridostigmine compositions of the disclosure provide 24-hour symptom control, tolerability, and reduced pill-burden by maintaining therapeutic plasma concentration of pyridostigmine over the 24-hour dosing period. In certain embodiments, the extended release pyridostigmine compositions of the disclosure provide 24-hour symptom control, e.g., providing nighttime and early morning functionality, by maintaining therapeutic plasma levels during the 24-hour dosing period. In certain embodiments, the extended release pyridostigmine compositions of the disclosure provide residual plasma levels of the drug in the morning, such that patients wake up feeling more refreshed and more functional before taking the morning dose, as compared with the currently marketed pyridostigmine products.

In certain embodiments, the extended release pyridostigmine compositions of the disclosure provide lower Fluctuation Index compared to marketed pyridostigmine products, e.g., MESTINON and MESTINON TIMESPAN®, to provide a 24-hour release profile with superior all day coverage and improved tolerability compared to the marketed pyridostigmine products. In certain embodiments, the gastroretentive dosage forms of the present disclosure, upon single dose administration, provide extended release of pyridostigmine bromide, with a Fluctuation Index of from about 0.1 to about 1, for at least about 8 hours, e.g., for up to about 24 hours.

In certain embodiments, the extended release pyridostigmine compositions of the disclosure, under fed conditions, provide higher Cmin/trough level compared to the marketed pyridostigmine products, e.g., MESTINON and MESTINON TIMESPAN®. In certain embodiments, the Cmin, under fed conditions is at least about 10 ng/ml. In certain embodiments, the Cmin, under fed conditions is at least about 15 ng/ml. In certain embodiments, Cmin, under fed conditions, is from 20 ng/ml to 30 ng/ml. In certain embodiments, Cmin under fed conditions is about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, or any intermediate values therein. In certain embodiments, the extended release pyridostigmine compositions of the disclosure reduce worsening of symptoms and improve quality of life by increasing the Cmin/trough levels. In certain embodiments, higher Cmin/trough level of extended release pyridostigmine compositions of the disclosure, compared to the marketed pyridostigmine products, e.g., MESTINON and MESTINON TIMESPAN®, reduce wearing off effects and worsening of symptoms, e.g., provide a better 24-hour control of Myasthenia Gravis symptoms.

In certain embodiments, the extended release pyridostigmine compositions of the disclosure provide lower Cmax/blunted Cmax compared to the marketed pyridostigmine products, e.g., MESTINON® and MESTINON® TIMESPAN®. In certain embodiments, the Cmax, under fed conditions is less than 60 ng/ml. In certain embodiments, Cmax, under fed conditions, is from 20 ng/ml to 50 ng/ml. In certain embodiments, the Cmax, under fed conditions is about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, or any intermediate values therein. In certain embodiments, the extended release pyridostigmine compositions of the disclosure improve tolerability and reduce side effects by reducing the Cmax and Fluctuation Index. In certain embodiments, lower Cmax of extended release pyridostigmine compositions of the disclosure, compared to the marketed pyridostigmine products, e.g., MESTINON® and MESTINON® TIMESPAN®, reduce cholinergic side effects, e.g., muscle cramps, and provide better 24-hour control of Myasthenia Gravis symptoms. In certain embodiments, the gastroretentive pyridostigmine compositions of the disclosure provide a Cmax:Cmin ratio of from 1 to 2. In certain embodiments, the gastroretentive pyridostigmine compositions of the disclosure provide a Cmax:Cmin ratio of about 1, about 1.1, about 1.2, about 1.3, about 1.4, about 1.5, about 1.6, about 1.7, about 1.8, about 1.9, about 1, or any intermediate values therein.

In certain embodiments, the extended release pyridostigmine compositions of the disclosure reduce pill-burden compared to the approved pyridostigmine compositions, e.g., MESTINON® and MESTINON® TIMESPAN®. According to the prescribing information for MESTINON®, the average daily dose of pyridostigmine is ten 60 mg tablets, ten teaspoons of suspension, or between one and three 180 mg ER tablets, spaced to provide maximum relief. The ER 180 mg tablets are administered, as 1-3 tablets, depending upon severity of the condition, once- or twice-daily with an interval of at least 6 hours between doses. Additionally, immediate release pyridostigmine tablets or oral solution may be required in conjunction with MESTINON® TIMESPAN® therapy. Such dosing regimens are challenging for patient adherence, which results in “wearing off” effect, symptom worsening, and acute cholinergic side effects. The extended release compositions of pyridostigmine compositions of the disclosure are suitable for once-daily dosing, thereby significantly reducing the pill-burden compared to marketed pyridostigmine products, e.g., MESTINON® and MESTINON® TIMESPAN®.

In certain embodiments, the gastroretentive pyridostigmine dosage forms of the present disclosure comprises an immediate release layer and an extended release component, wherein both the immediate release layer and the extended release component contain pyridostigmine or a pharmaceutically acceptable salt thereof, and wherein the dosage form provides an extended release, with reduced initial burst release, of pyridostigmine or a pharmaceutically acceptable salt thereof, for at least about 14 hours.

The extended release compositions described herein comprise pyridostigmine or pharmaceutically acceptable salts thereof. Nonlimiting pharmaceutically acceptable salts include hydrochloride, hydrobromide, hydroiodide, bromide, sulfite, sulfate, bisulfate, nitrate, salicylate, citrate, tartrate, bitartrate, lactate, phosphate, malate, maleate, fumarate, succinate, acetate, and pamoate salts. In certain embodiments, the pharmaceutically acceptable salt is bromide.

In certain embodiments, pyridostigmine or a pharmaceutically acceptable salt is present in amounts of from about 5 mg to about 500 mg per dose, and any other range in between. In certain embodiments, a pyridostigmine or a pharmaceutically acceptable salt thereof can be present in amounts from about 60 mg to about 450 mg, 60 mg to about 400 mg, from about 60 mg to about 360 mg, from about 60 mg to about 300 mg, from about 60 mg to about 240 mg, from about 60 mg to about 180 mg, or from about 60 mg to about 120 mg per dose, and any other range in between. In certain embodiments, a pyridostigmine or a pharmaceutically acceptable salt thereof can be present in an amount of about 60 mg, about 80 mg, about 105 mg, about 150 mg, about 205 mg, about 250 mg, about 275 mg, about 300 mg, about 340 mg, about 350 mg, about 400 mg, or any intermediate values therein, per dose to provide a wide range of doses depending on the disease severity. In certain embodiments, the pyridostigmine or a pharmaceutically acceptable salt thereof is present in an immediate release layer and an extended release component. In certain embodiments, the immediate release layer contains between about 0 mg and about 60 mg of a pyridostigmine or a pharmaceutically acceptable salt thereof. In certain embodiments, the extended release component contains between about 10 mg and about 500 mg of a pyridostigmine or a pharmaceutically acceptable salt thereof.

In certain embodiments, the pyridostigmine salt is pyridostigmine bromide. In certain embodiments, pyridostigmine bromide is present in amounts of from about 5 mg to about 500 mg per dose, and any other range in between. In certain embodiments, pyridostigmine bromide can be present in amounts from about 60 mg to about 450 mg, 60 mg to about 400 mg, from about 60 mg to about 360 mg, from about 60 mg to about 300 mg, from about 60 mg to about 240 mg, from about 60 mg to about 180 mg, or from about 60 mg to about 120 mg per dose, and any other range in between. In certain embodiments, a pyridostigmine or a pharmaceutically acceptable salt thereof can be present in an amount of about 60 mg, about 80 mg, about 105 mg, about 150 mg, about 205 mg, about 250 mg, about 275 mg, about 300 mg, about 340 mg, about 350 mg, about 400 mg, or any intermediate values therein, per dose to provide a wide range of doses depending on the disease severity. In certain embodiments, the pyridostigmine bromide is present in an immediate release layer and an extended release component. In certain embodiments, the immediate release layer contains between about 0 mg and about 60 mg of pyridostigmine bromide. In certain embodiments, the extended release component contains between about 10 mg and about 500 mg of pyridostigmine bromide.

In certain embodiments, the compositions of the disclosure can be administered QD as a single dosage unit. In certain embodiments, the compositions of the disclosure can be administered QD as multiple dosage units (e.g., two, three, or four dosage units). In certain embodiments, the compositions of the disclosure are suitable for twice-a-day dosing.

In certain embodiments, the present disclosure provide for a horizontally compressed, oval-shaped gastroretentive tablet dosage form containing a long axis and a short axis, wherein the long axis is between about 12 mm and about 22 mm, and the short axis is between about 8 mm and about 11 mm, and wherein the tablet, when in contact with media simulating gastric conditions, floats in about 30 minutes or less, and expands in about 60 minutes or less to a size that prevents its passage through a pyloric sphincter of a human.

In certain embodiments, the tablets maintain their GRS attributes of flotation and expansion for at least about 14 hours, at least about 16 hours, at least about 18 hours, at least about 24 hours, or any intermediate time periods therein. In certain embodiments, the tablets in a fully expanded state can withstand compression forces of about 10 N until about 14 hours, and after about 20 hours the matrix core can be squeezed even with a compression force of less than about 5 N.

### 6.2.1. Matrix Tablets

In certain embodiments, the extended release pyridostigmine compositions of the disclosure can be formulated as a matrix tablet comprising a rate controlling matrix core coated with a rate controlling functional coat/membrane, e.g., membrane-controlled matrix.

In certain embodiments, the matrix tablet of the disclosure can comprise a rate-controlling matrix core coated with a rate-controlling functional coat/membrane, e.g., a membrane-controlled matrix core. In certain embodiments, the matrix tablet of the disclosure can comprise a rate-controlling matrix core, a seal coat over the matrix core, a functional coat/membrane over the seal coat, a second seal coat over the functional coat, an immediate release layer over the seal coat, and an over coat/aesthetic coat over the immediate release layer. In certain embodiments, the matrix tablet can exclude an immediate release layer. In particular embodiments, in the absence of an immediate release layer, the over coat is the outermost coat.

In certain embodiments, the matrix core can be made by dry granulation. In certain embodiments, the matrix core can comprise pyridostigmine bromide, and at least one water-insoluble pH-independent lipophilic material. In certain embodiments, the matrix tablets can comprise pyridostigmine bromide and at least one swellable water-soluble hydrophilic polymer. As matrix tablets can be susceptible to sticking and mottling due to the hygroscopic nature of pyridostigmine bromide, the matrix tablets of the present disclosure can include an over coat to reduce the exposure of pyridostigmine bromide to moisture. In certain embodiments, the over coat can be the outermost coat. In certain embodiments, the release rate of pyridostigmine bromide from the matrix tablets of the disclosure can be controlled by varying the amount of lipophilic material in the matrix core and the composition of the functional coat over the matrix core. In certain embodiments, the release rate of pyridostigmine bromide from the compositions of the disclosure can be controlled by adjusting the coating level of the functional coat over the matrix core. In certain embodiments, water-insoluble material in the matrix core reduces drug dissolution and provides extended release of the drug, without initial burst release, for extended periods of time. In certain embodiments, the water-insoluble material can enhance compressibility of the composition. In certain embodiments, the water-insoluble material can include, but is not limited to, ethyl acrylate and methyl methacrylate copolymer (EUDRAGIT® NE, EUDRAGIT® NM), ammonio methacrylate copolymer (EUDRAGIT® RL 100, EUDRAGIT® RS 100, EUDRAGIT® RL PO, EUDRAGIT® RS PO), carnauba wax, stearic acid, ethylcellulose (ETHOCEL™), cellulose acetate, and silicon dioxide.

In certain embodiments, the matrix core can further comprise glidants, lubricants, compression aids, and fillers.

In certain embodiments, the disclosed matrix tablets can comprise one or more glidant materials to improve the flow of granules, and help to minimize the dosage form from weight variations. In certain embodiments, the glidants include, but are not limited to, silicon dioxide (SYLOID® 244FP), fumed silica (CAB-O-SIL®), talc, kaolin, or any combinations thereof.

In certain embodiments, the disclosed matrix tablets can comprise diluents and/or fillers. In certain embodiments, the diluents and/or fillers include, but are not limited to, lactose monohydrate USP, anhydrous lactose USP, directly compressible starches, hydrolyzed starches, pregelatinized starch, microcrystalline cellulose, silicified microcrystalline cellulose, carboxymethylcellulose and other cellulose polymers, sucrose and sucrose-based materials, dextrose, dibasic calcium phosphate anhydrous, dibasic calcium phosphate dihydrate, tricalcium phosphate, calcium sulfate dihydrate, and other alkaline inorganic salts, sugar alcohols such as mannitol (e.g., PARTECK® M200, MANNOGEM® XL), sorbitol, and xylitol, and confectioner's sugar.

In certain embodiments, diluents and/or fillers can be used as compression aids. In certain embodiments, diluents and/or fillers that can be used as compression aids include, but are not limited to, microcrystalline cellulose, silicified microcrystalline cellulose, and mannitol (e.g., PARTECK® M200, MANNOGEM® XL). In certain embodiments, the diluent and/or filler can be used in an amount of less than about 30% w/w of the tablet core. In certain embodiments, the diluent and/or filler can be present in an amount of from about 10% to about 40% w/w of the tablet. In certain embodiments, the diluent and/or filler can be present in an amount of less than about 25% w/w, less than about 24% w/w, less than about 23% w/w, less than about 22% w/w, less than about 21% w/w, less than about 20% w/w, less than about 15% w/w, less than about 10% w/w, less than about 5% w/w, or less than about 2.5% w/w of the total weight of the tablet core, or intermediate values thereof.

In certain embodiments, the matrix core can also include one or more lubricants. Lubricants are hydrophobic substances that decrease friction at the interface between a tablet's surface and the die wall during ejection and reduce wear on punches and dies. Lubricants enhance product flow by reducing interparticulate friction. In certain embodiments, the one or more lubricants can be, but are not limited to, magnesium stearate, stearic acid, calcium soaps, zinc stearate, polyoxyethylene monostearate, solid polyethylene glycols, calcium silicate, colloidal silicon dioxide, hydrogenated vegetable oils and fats, glyceryl monostearate, palmitic acid, talc, carnauba wax, mineral oil, polyethylene glycol, glyceryl palmitostearate, sodium benzoate, sodium stearyl fumarate, and any combination thereof. In certain embodiments, the lubricant is magnesium stearate. In certain embodiments, the lubricant can be present in an amount of from about 0.1% w/w to about 5% w/w based on the total weight of the matrix core. In certain embodiments, the lubricant can be present in an amount of less than about 4% w/w, less than about 3% w/w, less than about 2% w/w, less than about 1.5% w/w, less than about 1.4% w/w, less than about 1.3% w/w, less than about 1.2% w/w, less than about 1.1% w/w, or less than about 1.0% w/w based on the total weight of the matrix core.

In certain embodiments, the drug release can be controlled by a matrix-controlled membrane, e.g., a matrix core and functional coat over the matrix core. In certain embodiments, the drug release can be controlled by the functional coat/membrane. In certain embodiments, the matrix core can contain rate controlling water-insoluble material selected from a group comprising, but not limited to, ethyl acrylate and methyl methacrylate copolymer (EUDRAGIT® NE, EUDRAGIT® NM), copolymer of ethyl acrylate, methyl methacrylate, and trimethylammonioethyl methacrylate chloride (e.g., EUDRAGIT® RL 100, EUDRAGIT® RS 100, EUDRAGIT® RL PO, EUDRAGIT® RS PO, EUDRAGIT® RS 30D, EUDRAGIT® RL 30D), carnauba wax, stearic acid, ethylcellulose (ETHOCEL™), cellulose acetate, and silicon dioxide. In certain embodiments, the matrix core can contain rate-controlling swellable water-soluble hydrophilic polymer selected from the group comprising, but not limited to, hydroxypropyl methylcellulose (BENECEL™ K4M PH DC), hydroxypropyl methylcellulose (METHOCEL™ K100 Premium LVCR/LVDC), a polyethylene oxide polymer, a carbomer, sodium alginate, or mixtures thereof. In certain embodiments, the swellable water-soluble hydrophilic polymer can be BENECEL™ K4M PH DC. In certain embodiments, the water-soluble hydrophilic polymer can be METHOCEL™ K100 Premium LVCR/LVDC. In certain embodiments, the water-soluble hydrophilic polymer can be a mixture of METHOCEL™ K100 Premium LVCR/LVDC and BENECEL™ K4M PH DC.

In certain embodiments, the functional coat can contain rate controlling water-insoluble material. In certain embodiments, the rate controlling polymers in the functional coat can comprise, but are not limited to, ethyl acrylate and methyl methacrylate copolymer (EUDRAGIT® NE, EUDRAGIT® NM), copolymer of ethyl acrylate, methyl methacrylate, and trimethylammonioethyl methacrylate chloride (EUDRAGIT® RL 100, EUDRAGIT® RS 100, EUDRAGIT® RL 30D, EUDRAGIT® RS 30D, EUDRAGIT® RL PO, EUDRAGIT® RS PO), carnauba wax, stearic acid, ethylcellulose (ETHOCEL™), cellulose acetate, and polyvinyl acetate dispersion (KOLLICOAT® SR). In certain embodiments, the functional coat can further comprise a water-soluble pore former. In certain embodiments, the water-soluble pore former can include, but is not limited to, polyethylene glycol (PEG 400, PEG 1000, PEG 1450, PEG 3350), hydroxypropyl cellulose, polyvinyl pyrolidone (PVP), KOLLIDON® 30, KOLLICOAT® IR, mannitol, and methylcellulose (METHOCEL™ E3, METHOCEL™ E5, METHOCEL™ E6).

In certain embodiments, the matrix core and the functional coat over the matrix core can include stearic acid, ethylcellulose, cellulose acetate, and/or silicon dioxide to control the release of pyridostigmine bromide. In certain embodiments, the matrix core can be at least partially covered with the functional coat. In certain embodiments, the functional coat can completely surround the matrix core.

In certain embodiments, the matrix tablet can further include a seal coat between the matrix core and the functional coat. In certain embodiments, the seal coat can cover at least a portion of the matrix core. In certain embodiments, the seal coat can comprise a nonionic water-soluble polymer. In certain embodiments, the nonionic water-soluble polymer can be selected from the group consisting of a polyvinyl alcohol-based polymer, methyl cellulose, hydroxyethyl cellulose, hydroxypropyl cellulose, hydroxypropyl methylcellulose, and mixtures thereof.

In certain embodiments, the matrix tablet can further include an over coat. In certain embodiments, the over coat can cover at least a portion of the functional coat. In certain embodiments, the over coat can completely cover the functional coat. In certain embodiments, the over coat can comprise one or more water-soluble hydrophilic polymers selected from the group consisting of water-soluble polymer selected from a group consisting of a polyvinyl alcohol-based polymer (e.g., Opadry® II), methyl cellulose, hydroxyethyl cellulose, hydroxypropyl cellulose, hydroxypropyl methylcellulose, and mixtures thereof. In certain embodiments, the water-soluble hydrophilic polymers in the over coat can include polyvinyl alcohol and polyethylene glycol, e.g., Opadry® White.

In certain embodiments, the matrix core can be further coated with an immediate release drug layer comprising pyridostigmine bromide. In certain embodiments, the dosage form comprises a matrix core, a functional coat covering at least a portion of the matrix core, a seal coat covering at least a portion of the functional coat, an IR drug layer covering at least a portion of the seal coat, and an over coat/aesthetic coat covering at least a portion of the IR drug layer.

In certain embodiments, the matrix tablets can comprise a matrix core and a functional coat. In certain embodiments, the matrix core can comprise one or more of pyridostigmine bromide, stearic acid, carnauba wax, ethylcellulose, silicon dioxide, fumed silica, mannitol, magnesium stearate and combinations thereof. In certain embodiments, the matrix core can comprise from about 100 mg to about 250 mg, from about 150 mg to about 200 mg, or about 180 mg of pyridostigmine bromide. In certain embodiments, the matrix core can further optionally comprise from about 20 mg to about 200 mg, from about 50 mg to about 180, or about 90 mg of stearic acid. In certain embodiments, the matrix core can further optionally comprise from about 50 mg to about 200 mg, or from about 80 mg to about 160 mg of carnauba wax. In certain embodiments, the matrix core can further optionally comprise from about 50 mg to about 150 mg, or about 100 mg of ethylcellulose. In certain embodiments, the matrix core can further optionally comprise from about 20 mg to about 250 mg, from about from about 50 mg to about 200 mg, or about 180 mg of silicon dioxide. In certain embodiments, the matrix core can further optionally comprise from about 5 mg to about 40 mg, from about 10 mg to about 25 mg, or about 20 mg of fumed silica. In certain embodiments, the matrix core can further comprise from about 50 mg to about 200 mg, from about 75 mg to about 150 mg, or about 100 mg of mannitol. In certain embodiments, the matrix core can further comprise from about 1 mg to about 10 mg, from about 3 mg to about 7 mg, or about 5 mg of magnesium stearate. In certain embodiments, the matrix tablet comprises a functional coat. In certain embodiments, the functional coat can comprise one or more of cellulose acetate, polyethylene glycol, methylcellulose, and combinations thereof. In certain embodiments, the functional coat can comprise from about 10 mg to about 70 mg, from about 30 mg to about 65 mg, or from about 40 mg to about 50 mg of cellulose acetate. In certain embodiments, the functional coat can further comprise from about 1 mg to about 10 mg, from about 1.5 mg to about 7 mg, or from about 2 mg to about 5 mg of polyethylene glycol. In certain embodiments, the functional coat can further comprise from about 2 mg to about 10 mg, from about 3 mg to about 7 mg, or from about 3 mg to about 5 mg of methylcellulose.

### 6.2.2. Gastroretentive Tablets

In embodiments, the extended release pyridostigmine compositions can be formulated as gastroretentive tablets that provide a constant reservoir for continuous absorption of pyridostigmine in the proximal gastrointestinal tract and provide constant levels of pyridostigmine or a pharmaceutically acceptable salt thereof over extended periods of time. The sustained release profile with fewer fluctuations in the plasma concentration is expected to fulfill an unmet need by reducing the frequency of dosing while providing better control of symptoms and improved tolerability (e.g., decreased side effects, including unwanted GI side effects) compared to currently marketed pyridostigmine products. The gastroretentive compositions (e.g., tablets) of the disclosure are particularly suitable for long-term treatment of mild to moderate MG, and as an adjunct therapy in patients who are also receiving steroids and immunotherapy. In certain embodiments, the gastroretentive tablets of the disclosure can provide gastric retention and continuous release of pyridostigmine, without initial dose dumping of pyridostigmine, for at least about 14 hours, e.g., about 24 hours.

In certain embodiments, the gastroretentive tablets of the disclosure can comprise an expanding core and a permeable elastic membrane surrounding the core, wherein the core and the membrane together can provide controlled extended release, with minimized (e.g., eliminated) or reduced dose dumping/initial burst release, of pyridostigmine bromide for at least about 14 hours.

In certain embodiments, the gastroretentive tablets of the disclosure can comprise an immediate release layer and an extended release component. The immediate release layer can comprise pyridostigmine or a pharmaceutically acceptable salt thereof, and the extended release component can comprise a core coated with a permeable elastic membrane. In certain embodiments, the immediate release layer can provide a drug plasma concentration that is sufficient to overcome the lag time in pyridostigmine release seen without application of an IR layer, and sufficient to provide instant therapeutic effects, with possible reduced or eliminated GI side effects, and the extended release component can provide controlled extended release of the drug for a period of at least about 14 hours.

In certain embodiments, the gastroretentive tablets of the disclosure, when in contact with simulated gastric fluid, can float in about 40 minutes or less. In certain embodiments, gastroretentive compositions of the disclosure float in 40 minutes or less on coming in contact with 200 ml dissolution medium comprising 50 mM of pH 4.5 acetate buffer with 100 mM NaCl, measured using rotating bottle method at 5 rpm and 37° C. In certain embodiments, gastroretentive compositions of the disclosure, comprising a functional coating weight gain from about 200 mg to about 20 mg, based on the core weight without the functional coat, exhibit from about 150% to about 410% volume gain on flotation, measured using rotating bottle method at 5 rpm and 37° C., in 200 ml of 50 mM pH 4.5 acetate buffer containing 10 mM of NaCl. In certain embodiments, gastroretentive compositions of the disclosure, comprising a functional coating weight gain of about 200 mg, based on the core weight without the functional coat, exhibit from about 250% to about 810% volume gain at 90 minutes from the time of administration into 200 ml dissolution medium comprising 50 mM pH 4.5 acetate buffer containing 10 mM of NaCl, measured using rotating bottle method at 5 rpm and 37° C. In certain embodiments, gastroretentive compositions of the disclosure, comprising a functional coating weight gain of about 250 mg, based on the core weight without the functional coat, exhibit from about 250% to about 550% volume gain at 60 minutes from the time of administration into 200 ml dissolution medium comprising 50 mM pH 4.5 acetate buffer containing 10 mM of NaCl, measured using rotating bottle method at 5 rpm and 37° C. In certain embodiments, the gastroretentive tablets of the disclosure on coming in contact with gastric fluid can float in about 60 minutes or less, expand in about 60 minutes or less to a size that prevents passage through the pyloric sphincter, and provide extended release of pyridostigmine for at least about 14 hours, e.g., about 24 hours.

### 6.2.2.1 Permeable Membrane/Functional Coat

The gastroretentive compositions (e.g., tablets) of the disclosure include a rapidly expanding membrane surrounding a hydrophilic core. In certain embodiments, the membrane is a water-insoluble, permeable elastic membrane surrounding the core. The permeable membrane allows the flow of gastric fluid into the composition, which initiates gas generation from gas-generating agents, and the membrane flexibility allows for rapid expansion and immediate flotation of the composition. In certain embodiments, the membrane comprises a plasticizer and at least one ammonium polymethacrylate copolymer (copolymer of ethyl acrylate, methyl methacrylate, and trimethylammonioethyl methacrylate chloride.

The ammonium polymethacrylate copolymer provides permeability of the membrane and the plasticizer improves elasticity and mechanical strength of the membrane. The plasticizer provides elasticity to the membrane, ensuring that the membrane does not rupture upon expanding and that the gastroretentive drug delivery system provides the desired characteristics for drug release, hydrodynamic balance, and mechanical strength to withstand variations in pH and shear in the stomach during fed and fasted conditions. In certain embodiments, as dissolution of the active agent in the core proceeds, the plasticizer can leach out of the membrane. In certain embodiments, leaching of the plasticizer can make the membrane brittle, such that the membrane does not remain intact and the dosage form can break into pieces by the end of drug release. Hydrophilic plasticizers suitable for the disclosure include, but are not limited to, glycerin, polyethylene glycols, polyethylene glycol monomethyl ether, propylene glycol, sorbitol sorbitan solution, and mixtures thereof. Hydrophobic plasticizers suitable for the disclosure include, but are not limited to, acetyl tributyl citrate, acetyl triethyl citrate, castor oil, diacetylated monoglycerides, dibutyl sebacate, diethyl phthalate, triacetin, tributyl citrate, triethyl citrate, gelucire 39/01, gelucire 43/01, and mixtures thereof. In certain embodiments, the plasticizers include various polyethylene glycols, glycerin, and/or triethyl citrate. In certain embodiments, the plasticizer is triethyl citrate.

In certain embodiments of the disclosure, the permeable elastic membrane comprises two (or more) water-insoluble polymers: at least one of EUDRAGIT® RL 30D (copolymer dispersion of ethyl acrylate, methyl methacrylate, and methacrylic acid ester with quaternary ammonium groups, 1:2:0.2) or EUDRAGIT® RS 30D (copolymer dispersion of ethyl acrylate, methyl methacrylate, and methacrylic acid ester with quaternary ammonium groups, 1:2:0.1) to improve permeability; and at least one of KOLLICOAT® SR 30D (dispersion of polyvinyl acetate and polyvinyl pyrolidone), EUDRAGIT® NE 30D (copolymer dispersion of ethyl acrylate, methyl methacrylate), or EUDRAGIT® NM 30D (copolymer dispersion of ethyl acrylate, methyl methacrylate), to improve mechanical strength (tensile strength). In certain embodiments, the membrane further comprises a hydrophilic polymer and, optionally, water-soluble nonionic polymer that act as a pore former, to modify its elasticity, permeability, and tensile strength.

In certain embodiments, the permeable elastic membrane provides desired characteristics for drug release and tensile strength to withstand peristalsis and mechanical contractility of the stomach (shear). The combination of a water-soluble hydrophilic polymer in the core, and the unique permeable elastic membrane formed over the tablet core by the coating of a homogeneous dispersion of at least one of EUDRAGIT® RL 30D or EUDRAGIT® RS 30D (copolymer of ethyl acrylate, methyl methacrylate, and trimethylammonioethyl methacrylate chloride) to improve permeability, and at least one of KOLLICOAT® SR 30D, EUDRAGIT® NE 30D, or EUDRAGIT® NM 30D (collectively “neutral polymethacrylate copolymer dispersions”) to improve mechanical strength (tensile strength), can provide the desired extended drug release while maintaining the integrity of the core in an expanded state, thus extending the gastric residence time and preventing the dosage form from being emptied from the stomach until substantial or complete release of the drug, usually after a prolonged period.

In certain embodiments, the water-insoluble polymers in the permeable elastic membrane comprises at least one of EUDRAGIT® RL PO r EUDRAGIT® RS PO (i.e., ammonium polymethacrylate copolymers in powder form). In certain embodiments, the permeable elastic membrane is formed over the core by coating the core with EUDRAGIT® RL PO (copolymer of ethyl acrylate, methyl methacrylate, and trimethylammonioethyl methacrylate chloride (1:2:0.2) with a glass transition temperature (T_{g}) of 63) and/or EUDRAGIT® RS PO (copolymer of ethyl acrylate, methyl methacrylate, and trimethylammonioethyl methacrylate chloride (1:2:0.1) with a glass transition temperature (T_{g}) of 65), a plasticizer, and talc.

In certain embodiments, the membrane comprises a water-insoluble polymer, a plasticizer, and at least one pore former comprising a water-soluble nonionic polymer. In certain embodiments, the pore formers and plasticizers modify membrane elasticity, permeability, and tensile strength. In certain embodiments, the membrane excludes any pore former. In certain embodiments, examples of insoluble permeable components of the permeable elastic membrane include, but are not limited to, copolymers of ethyl acrylate, methyl methacrylate, and trimethylammonioethyl methacrylate chlorides (e.g., EUDRAGIT® RL 100, EUDRAGIT® RS 100, EUDRAGIT® RL 30D, EUDRAGIT® RS 30D, EUDRAGIT® RL PO, EUDRAGIT® RS PO); cellulose acetate phthalate; ethyl cellulose; hypromellose acetate succinate, and mixtures thereof.

In certain embodiments, examples of insoluble components of the permeable elastic membrane that provide elasticity to the membrane include, but are not limited to, copolymers of ethyl acrylate and methyl methacrylate (e.g., EUDRAGIT® NE 30D, EUDRAGIT® NM 30D), polyvinyl acetates (e.g., KOLLICOAT® SR 30D), thermoplastic polyurethanes, ethylene-vinyl acetate, polydimethyl siloxane, and mixtures thereof.

In certain embodiments, the permeable elastic membrane is a coating of EUDRAGIT® RL PO and/or EUDRAGIT® RS PO. In certain embodiments, the core is coated with a coating solution/suspension/dispersion of EUDRAGIT® RL PO and/or EUDRAGIT® RS PO in acetone and water mixture.

In certain embodiments, the coating dispersion can include at least one of EUDRAGIT® RL PO or EUDRAGIT® RS PO to improve permeability, and at least one plasticizer to improve mechanical strength (tensile strength). In certain embodiments, powder forms of EUDRAGIT®, e.g., EUDRAGIT® RL PO and EUDRAGIT® RS PO, are preferred over EUDRAGIT® dispersions, e.g., EUDRAGIT® RS 30D and EUDRAGIT® RL 30D.

In certain embodiments, the permeability of the permeable elastic membrane can be adjusted to provide a floating lag time of less than about 60 minutes and floating time of from about 1 hour to about 24 hours. In certain embodiments, the gastroretentive pyridostigmine tablets of the disclosure can comprise a membrane containing at least one copolymer of ethyl acrylate, methyl methacrylate, and trimethylammonioethyl methacrylate chloride, e.g., EUDRAGIT® RL 100, EUDRAGIT® RS 100, EUDRAGIT® RL 30D, EUDRAGIT® RS 30D, EUDRAGIT® RL PO, or EUDRAGIT® RS PO, and can exhibit a floating lag time of about 60 minutes or less and floating time of from about 1 hour to about 24 hours. In certain embodiments, the ammonium polymethacrylate copolymer can be present in an amount of from about 70% to about 95% w/w of the membrane composition to provide desired permeability of the membrane. In certain embodiments, plasticizer can be present in an amount of from about 5 wt % to about 25 wt %, from about 10 wt % to about 20 wt %, from about 10 wt % to about 15 wt %, and any intermediate ranges there in, of the membrane composition to provide desired tensile strength, and elasticity for rapid expansion of the membrane. In certain embodiments, the plasticizer is present in an amount of at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, and at least about 25 wt % of the membrane composition.

In certain embodiments, the membrane can further include an anti-tacking agent selected from the group consisting of talc, colloidal silicon dioxide, magnesium trisilicate, powdered cellulose, starch, and tribasic calcium phosphate. In certain embodiments, the anti-tacking agent can be colloidal silicon dioxide and/or talc. In certain embodiments, the anti-tacking agent can be present in an amount of from about 5 wt % to about 30 wt % of the membrane composition. In certain embodiments, the anti-tacking agent is present in an amount of about 5 wt %, about 10 wt %, about 15 wt %, about 20 wt %, about 25 wt %, about 30 wt %, or any intermediate values therein, by weight of the membrane composition.

In certain embodiments, the membrane can expand the dosage form in about 60 minutes to a size that prevents its passage through the pyloric sphincter; and the hydrophilic core, surrounded by the membrane, can swell with imbibition and absorption of fluid and assist the membrane in providing an extended release of the drug. In certain embodiments, the membrane can be highly elastic and flexible due to the presence of at least one plasticizer and/or a copolymer of ethyl acrylate and methyl methacrylate and can expand rapidly with an outward pressure on the membrane from the generated CO₂ gas.

In certain embodiments, the membrane can provide extended release of the drug for at least about fourteen hours, e.g., about twenty-four hours.

### 6.2.2.2 Core

In certain embodiments, the core can comprise pyridostigmine or a pharmaceutically acceptable salt thereof (e.g., pyridostigmine bromide), an acid, a gas-generating agent, and at least one swellable water-soluble hydrophilic polymer. In certain embodiments, the core further comprises a filler and/or a wicking agent,

In certain embodiments, the core comprises from about 10 mg to about 500 mg of pyridostigmine or a pharmaceutically acceptable salt thereof. In certain embodiments, the core comprises from about 100 mg to about 350 mg, from about 150 mg to about 300 mg, from about 200 mg to about 250 mg, or any intermediate ranges therein, of pyridostigmine or a pharmaceutically acceptable salt thereof. In certain embodiments, the core comprises from about 50 mg to about 100 mg, from about 75 mg, about 125 mg, from about 100 mg to about 150 mg, from about 125 mg to about 175 mg, from about 75 mg to about 200 mg, from about 100 mg to about 225 mg, from about 125 mg to about 250 mg, from about 150 mg to about 275 mg, from about 175 mg to about 300 mg, from about 200 mg to about 325 mg, from about 225 mg to about 350 mg, from about 275 mg to about 375 mg, from about 300 mg to about 400 mg, or any intermediate values therein, of pyridostigmine or a pharmaceutically acceptable salt thereof.

In certain embodiments, the core comprises a swellable water-soluble hydrophilic polymer in an amount of from about 5 wt % to about 35 wt %, based on the total weight of the core. In certain embodiments, the core comprises a swellable water-soluble hydrophilic polymer selected from the group comprising hydroxypropyl methylcellulose, hydroxypropyl cellulose, methyl cellulose, a polyethylene oxide polymer, a carbomer, sodium alginate, and mixtures thereof. In particular embodiments, the swellable water-soluble hydrophilic polymer is hydroxypropyl methylcellulose. In certain embodiments, the swellable water-soluble hydrophilic polymer in the core comprises a high viscosity hydroxypropyl methylcellulose/hypromellose with a viscosity of from about 2,500 mPa·s to about 300,000 mPa·s, and a weight average molecular weight of from about 350,000 to about 1,500,000 (e.g., BENECEL™ K4M PH DC with a viscosity of from about 2,700 mPa·s to about 5,040 mPa·s); a low viscosity hydroxypropyl methylcellulose/hypromellose with a viscosity of from about 50 mPa·s to about 2,400 mPa·s, and a weight average molecular weight of from about 150,000 to about 300,000 (e.g., METHOCEL™ K100 Premium LVCR/LVDC with a viscosity of from about 80 mPa·s to 120 mPa·s), a polyethylene oxide polymer, a carbomer, sodium alginate, or mixtures thereof. In certain embodiments, the swellable water-soluble hydrophilic polymer comprises hypromellose 2208 with a viscosity of from about 80 mPa·s to 120 mPa·s (METHOCEL™ K100 Premium LVCR/LVDC). In certain embodiments, the swellable water-soluble hydrophilic polymer comprises a mixture of two or more hypromelloses with different viscosities, e.g., METHOCEL™ K100 Premium LVCR/LVDC and BENECEL™ K4M PH DC. In certain embodiments, the low viscosity hypromellose has a viscosity of from about 80 mPa·s to about 120 mPa·s, from about 200 mPa·s to about 300 mPa·s, from about 562 mPa·s to about 1050 mPa·s, from about 1,125 mPa·s to about 2,100 mPa·s, or any intermediate ranges therein. In certain embodiments, the low viscosity hypromellose has a weight average molecular weight of about 164,000, about 200,000, about 250,000, about 300,000, or any intermediate values therein. In certain embodiments, the high viscosity hypromellose has a viscosity of from about 2,700 mPa·s to about 5,040 mPa·s, from about 13,500 mPa·s to about 25,200 mPa·s, from about 26,250 mPa·s to about 49,000 mPa·s, from about 75,000 mPa·s to about 140,000 mPa·s, from about 150,000 mPa·s to about 280,000 mPa·s, or any intermediate ranges therein. In certain embodiments, the high viscosity hypromellose has a weight average molecular weight of about 400,000, about 575,000, about 675,000, about 1,000,000, about 1,200,000, or any intermediate values therein. In certain embodiments, the water-soluble hydrophilic polymer is present in an amount of from about 5 wt % and about 35 wt %, based on the total weight of the core. In certain embodiments, the hypromellose 2208, with a viscosity of from about 2,700 mPa·s to about 5,040 mPa·s, is present in an amount of from about 5 wt % to about 35 wt %, based on the total weight of the core. In certain embodiments, hypromellose 2208, with a viscosity of from about 2,700 mPa·s to about 5,040 mPa·s, is present in an amount of about 5 wt %, about 10 wt %, about 15 wt %, about 20 wt %, about 25 wt %, about 30 wt %, about 35 wt %, or any intermediate values therein, based on the total weight of the core. In certain embodiments, the dosage form comprises a low viscosity hypromellose 2208 with a viscosity of from about 80 mPa·s to about 120 mPa·s (e.g., METHOCEL K100 Premium LVCR/LVDC). In certain embodiments, METHOCEL K100 Premium LVCR/LVDC is present in an amount of about 5 wt %, about 10 wt %, about 15 wt %, about 20 wt %, about 25 wt %, about 30 wt %, about 35 wt %, or any intermediate values therein, based on the total weight of the core. In certain embodiments, the mixture of METHOCEL™ K100 Premium LVCR/LVDC and BENECEL™ K4M PH DC in the dosage form provides extended release of pyridostigmine bromide for about 14 hours or more. In certain embodiments, the presence of hypromellose 2208 in an amount of between about 20% w/w and about 35% w/w of the core, helps in providing extended release of the drug for at least about 14 hours. In certain embodiments, the presence of METHOCEL™ K100 Premium LVCR/LVDC in the mixture of two or more hypromellose 2208 aids in the complete emptying of the dosage form at the end of the drug release period.

In certain embodiments, the core comprises gas-generating agents that can generate CO₂ on interaction with acid. Examples of gas-generating agents that can be used in the compositions of the present disclosure include, but are not limited to, all organic and inorganic strong and weak bases, e.g., carbonate and bicarbonate salts of alkali and alkaline earth metals, that can interact with stomach acid for in situ gas generation. In certain embodiments, the gas-generating agent can be sodium bicarbonate, sodium carbonate, magnesium carbonate, and/or calcium carbonate. In certain embodiments, a mixture of calcium carbonate and sodium bicarbonate can provide desired sustained release of CO₂. In certain embodiments, the gas-generating agent is present in an amount of from about 5 wt % to about 50 wt %, based on the total weight of the core. In certain embodiments, the gas-generating agent can be present in an amount of about 5 wt %, about 10 wt %, about 15 wt %, about 20 wt %, about 25 wt %, about 30 wt %, about 35 wt %, about 40 wt %, about 45 wt %, about 50 wt %, or any intermediate values therein, based on the total weight of the core.

In certain embodiments, the core can comprise an acid to achieve rapid floating and expansion of the tablet, regardless of the gastric pH. In certain embodiments, the acids include, but are not limited to, organic acids selected from the group comprising succinic acid, citric acid, acetic acid, malic acid, fumaric acid, stearic acid, tartaric acid, boric acid, benzoic acid, or mixtures thereof. In certain embodiments, the acid can be succinic acid. In certain embodiments, the acid can be present in an amount of between 0 wt % and about 20 wt % of the core. In certain embodiments, the acid can be present in an amount of about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 12.5 wt %, about 15 wt %, about 20 wt %, or any intermediate values therein, based on the total weight of the core.

In certain embodiments, the acid is succinic acid and the gas-generating agent is a mixture of sodium bicarbonate and calcium carbonate. In certain embodiments, the gastroretentive dosage forms of the disclosure include equimolar amounts of acid and each of sodium bicarbonate and calcium carbonate (e.g., equimolar amounts of succinic acid and sodium bicarbonate, and equimolar amounts of succinic acid and calcium carbonate).

In certain embodiments, the core comprises a wicking agent/disintegrant selected from a group comprising, but not limited to, croscarmellose sodium; sodium starch glycolate; low-substituted hydroxypropyl cellulose; a mixture of 90% mannitol, 5% crospovidone, and 5% polyvinyl acetate (LUDIFLASH®); a coprocessed blend of mannitol, starch, crospovidone, croscarmellose sodium, colloidal silica, and silica (PHARMABURST®); microcrystalline cellulose; alginic acid; and mixtures thereof. In certain embodiments, the wicking agent is crospovidone. In certain embodiments, the wicking agent is present in an amount of about 5 wt % to about 25 wt %, based on the total weight of the core. In certain embodiments, the wicking agent is present in amount of about 5 wt %, about 10 wt %, about 15 wt %, about 20 wt %, about 25 wt %, or any intermediate values therein, based on the total weight of the core.

In certain embodiments, the core further comprises at least one lubricant selected from the group comprising magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof. In certain embodiments, the lubricant is magnesium stearate. In certain embodiments, the lubricant is present in an amount of about 0.1 wt % to about 2 wt % of the core. In certain embodiments, the lubricant is present in an amount of about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, or any intermediate values therein, based on the total weight of the core.

In certain embodiments, the core comprises at least one glidant selected from the group comprising talc, colloidal silicon dioxide, magnesium trisilicate, powdered cellulose, starch, and tribasic calcium phosphate. In certain embodiments, the glidant can be colloidal silicon dioxide. In certain embodiments, the glidant can be present in an amount of about 0.1 wt % to about 2 wt %, based on the total weight of the core. In certain embodiments, the glidant is present in an amount of about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, or any intermediate values therein, based on the total weight of the core.

In certain embodiments, the core can further comprise a filler/compression aid. In certain embodiments, mannitol is used as a filler/compression aid. In certain embodiments, mannitol can be used as an osmotic agent. In certain embodiments, mannitol can be present in an amount of about 1 wt % to about 40 wt % based on the total weight of the core.

In certain embodiments, the core further comprises at least one color pigment. In certain embodiments, the core can include at least one pigment comprising iron oxide or lake-based colors. In certain embodiments, the pigment can be a lake-based color. In certain embodiments, the pigment can be an iron oxide pigment, e.g., oxide pigment black or Red blend. In certain embodiments, the pigment can be present in an amount of about 0.5 wt % to about 2 wt % based on the total weight of the core.

### 6.2.2.3 Immediate Release Drug Layer

In certain embodiments, the gastroretentive pyridostigmine compositions (e.g., tablets) of the disclosure can include an immediate release component comprising an immediate release drug layer; and an extended release component to provide a biphasic release of pyridostigmine or a pharmaceutically acceptable salt thereof. In certain embodiments, the immediate release layer can cover at least a portion of the functional coat/permeable membrane. In certain embodiments, there is a seal coat (seal coat-1) between functional coat and immediate release drug layer. In certain embodiments, the immediate release drug layer comprises pyridostigmine or a pharmaceutically acceptable salt thereof and a binder.

In certain embodiments, the immediate release layer comprises from about 0 mg to about 60 mg of pyridostigmine or a pharmaceutically acceptable salt thereof. In certain embodiments, the immediate release layer comprises about 1 mg, about 2 mg, about 3 mg, about 4 mg, 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, or any intermediate values therein. In certain embodiments, the binder(s) can be selected from the group consisting of, but not limited to, povidone K 90, hypromellose, starch, acacia, gellan gum, low viscosity hydroxypropyl cellulose, methylcellulose, sodium methylcellulose, polyvinyl alcohol, polyvinyl acetates (e.g., KOLLICOAT® SR), polyethylene oxide (e.g., POLYOX®), polyethylene glycol, alginates, pegylated polyvinyl alcohol, and mixtures thereof. In certain embodiments, the binder can be hydroxypropyl cellulose. In certain embodiments, the binders can be present in an amount of about 0.5 wt % to about 30 wt % of the amount of drug in the immediate release drug layer. In certain embodiments, the binders can be present in an amount of about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, about 10 wt %, about 15 wt %, about 20 wt %, about 25 wt %, about 30 wt %, or any intermediates values therein, of the amount of drug in the immediate release drug layer.

### 6.2.2.4 Additional Coats

In certain embodiments, the gastroretentive tablets of the disclosure further can include a seal coat between the core and the permeable elastic membrane, between the permeable elastic membrane and the immediate release drug layer, and/or between the immediate release drug layer and the cosmetic coat. In certain embodiments, the seal coat over the core can cover at least a portion of the core. In certain embodiments, the seal coat over the permeable elastic membrane can cover at least a portion of the membrane. In certain embodiments, the seal coat over the immediate release drug layer can cover at least a portion of the drug layer. In certain embodiments, the seal coat can comprise one or more water-soluble hydrophilic polymers selected from the group consisting of a polyvinyl alcohol-based polymer (OPADRY® white, OPADRY® clear), methyl cellulose, hydroxyethyl cellulose, hydroxypropyl cellulose, hydroxypropyl methylcellulose, and mixtures thereof. In certain embodiments, the seal coat can comprise hydroxypropyl cellulose. In certain embodiments, the seal coat can be present in an amount of about 0.5 wt % to about 5 wt % of the uncoated core, membrane-coated core, or drug layered core. In certain embodiments, the seal coat can be present in an amount of about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, or any intermediate values therein, of the uncoated core, membrane-coated core, or core with drug layer.

In certain embodiments, the gastroretentive tablets of the disclosure further includes an over coat/aesthetic coat/cosmetic coat. In certain embodiments, the over coat covers at least a portion of the permeable elastic membrane (in a composition without an IR drug layer) or a portion of drug layer (in a composition with an IR drug layer) or a portion of seal coat present over the drug layer. In certain embodiments, the over coat can completely cover the permeable elastic membrane or the IR drug layer or the seal coat (Seal coat-2) over the drug layer. In certain embodiments, the over coat can be the outermost coat. In certain embodiments, the over coat can comprise one or more water-soluble hydrophilic polymers selected from the group consisting of methylcellulose, hydroxymethyl cellulose, hydroxypropyl cellulose, hydroxypropyl methylcellulose, and polyvinyl alcohol-based OPADRY® white.

In certain embodiments, the gastroretentive tablets of the disclosure can include at least one laser-drilled orifice/hole that passes through the permeable elastic membrane/functional coat and seal coat-1. In certain embodiments, the gastroretentive dosage forms of the disclosure include multiple laser-drilled orifices/holes. In certain embodiments, the gastroretentive dosage forms of the disclosure do not include laser-drilled holes in the IR drug layer and the over coat (i.e., laser-drilled holes do not pass through these layers).

### 6.2.2.5 Compositions

In certain embodiments, the present disclosure provide for a gastroretentive dosage form comprising an immediate release layer containing pyridostigmine bromide, and an extended release component, wherein the extended release component comprises a core comprising pyridostigmine bromide, an acid, a gas-generating agent, and a water-soluble hydrophilic polymer that swells via imbibition of gastric fluid, and a permeable elastic membrane surrounding the core and comprising a plasticizer, and a highly permeable and elastic copolymer based on ethyl acrylate, methyl methacrylate, and trimethylammonioethyl methacrylate chloride, and wherein the dosage form provides an extended release, with reduced initial burst release, of pyridostigmine bromide, for at least about 14 hours. In certain embodiments, the core of the dosage form of the present disclosure includes a wicking agent selected from the group consisting of crospovidone; croscarmellose sodium; sodium starch glycolate; low-substituted hydroxypropyl cellulose; a mixture of mannitol, crospovidone, and polyvinyl acetate; a coprocessed blend of mannitol, starch, crospovidone, croscarmellose sodium, colloidal silica, and silica; microcrystalline cellulose; alginic acid; and mixtures thereof. In certain other embodiments, the core of the dosage form comprises crospovidone as a wicking agent. In certain embodiments, the dosage form of the present disclosure comprises a water-soluble hydrophilic polymer selected from the group consisting of hydroxypropyl methylcellulose, hydroxypropyl cellulose, methyl cellulose, a polyethylene oxide polymer, a carbomer, sodium alginate, and mixtures thereof. In particular embodiments, the water-soluble hydrophilic polymer is hydroxypropyl methylcellulose. In certain other embodiments, the water-soluble hydrophilic polymer is methyl cellulose. In certain other embodiments, the water-soluble hydrophilic polymer is a mixture of hydroxypropyl methylcellulose with an average molecular weight of greater than or equal to 164,000 and less than 400,000, and hydroxypropyl methylcellulose with an average molecular weight of between about 400,000 and about 1,200,000. In certain embodiments, the dosage form of the present disclosure comprises a gas-generating agent selected from the group consisting of NaHCO₃, CaCO₃, and a mixture thereof. In certain embodiments, the gas-generating agent is a mixture of NaHCO₃ and CaCO₃. In certain embodiments, the dosage form of the present disclosure comprises a plasticizer selected from the group consisting of triethyl citrate, triacetin, polyethylene glycol, propylene glycol, dibutyl sebacate, and mixtures thereof. In particular embodiments, the plasticizer is triethyl citrate. In certain embodiments, the permeable elastic membrane of the dosage form of the present disclosure is at least partially covered by the immediate release drug layer. In certain embodiments, the present disclosure provides for a dosage form that further includes a seal coat between the immediate release drug layer and the permeable elastic membrane. In certain embodiments, the seal coat of the dosage form or the present disclosure comprises a water-soluble polymer selected from the group consisting of a polyvinyl alcohol-based polymer, methyl cellulose, hydroxyethyl cellulose, hydroxypropyl cellulose, hydroxypropyl methylcellulose, or a mixture thereof. In certain embodiments, the dosage form of the present disclosure further includes an over coat over the immediate release drug layer. In particular embodiments, the over coat comprises a water-soluble polymer selected from a group consisting of a polyvinyl alcohol-based polymer, methyl cellulose, hydroxyethyl cellulose, hydroxypropyl cellulose, hydroxypropyl methylcellulose, or a mixture thereof.

In certain embodiments, the present disclosure provides for an extended release gastroretentive pyridostigmine tablet comprising an immediate release layer containing pyridostigmine bromide, and an extended release component, wherein the extended release component comprises a core comprising pyridostigmine bromide, an acid, a gas-generating agent, and a water-soluble hydrophilic polymer that swells via imbibition of gastric fluid; and a permeable elastic membrane, surrounding the core, comprising a plasticizer, and a copolymer based on ethyl acrylate, methyl methacrylate, and trimethylammonioethyl methacrylate chloride, and wherein the tablet is suitable for once daily administration and is administered as a single tablet/day. In certain embodiments, the tablet of present disclosure comprises 100 mg, 200 mg, 250 mg, 300 mg, or 350 mg of pyridostigmine bromide.

In certain embodiments, the water-soluble hydrophilic polymer of the tablet of the present disclosure is selected from the group consisting of hydroxypropyl methylcellulose, hydroxypropyl cellulose, methyl cellulose, a polyethylene oxide polymer, a carbomer, sodium alginate, and mixtures thereof. In certain embodiments, the gas-generating agent of the tablet of the present disclosure comprises NaHCO₃, CaCO₃, or a mixture thereof. In certain embodiments, the plasticizer of the tablet of the present disclosure is selected from the group consisting of triethyl citrate, triacetin, polyethylene glycol, propylene glycol, dibutyl sebacate, and mixtures thereof. In certain embodiments, the tablet of the present disclosure further includes a wicking agent selected from the group consisting of crospovidone; croscarmellose sodium; sodium starch glycolate; low-substituted hydroxypropyl cellulose; a mixture of mannitol, crospovidone, and polyvinyl acetate; a coprocessed blend of mannitol, starch, crospovidone, croscarmellose sodium, colloidal silica, and silica; microcrystalline cellulose; alginic acid; and mixtures thereof. In certain embodiments, the permeable elastic membrane of the tablet of the present disclosure is at least partially covered by the immediate release drug layer. In certain embodiments, the tablet of the present disclosure further includes a seal coat between the immediate release drug layer and the permeable elastic membrane. In certain embodiments, the seal coat of the tablet of the present disclosure comprises a water soluble polymer selected from the group consisting of a polyvinyl alcohol-based polymer, methyl cellulose, hydroxyethyl cellulose, hydroxypropyl cellulose, hydroxypropyl methylcellulose, or a mixture thereof. In certain embodiments, the present disclosure provides for a tablet that further includes an over coat over the immediate release drug layer. In certain embodiments, the overcoat of the tablets of the present disclosure comprises a water-soluble polymer selected from a group consisting of a polyvinyl alcohol-based polymer, methyl cellulose, hydroxyethyl cellulose, hydroxypropyl cellulose, hydroxypropyl methylcellulose, and mixtures thereof.

In certain embodiments, the pyridostigmine bromide gastroretentive tablets can comprise a core, a functional coat, and an overcoat. In certain embodiment, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, methylcellulose, fumed silica, magnesium stearate, and combinations thereof. In certain embodiments, the core of the pyridostigmine bromide gastroretentive tablets can comprise from about 100 mg to about 250 mg, from about 150 mg to about 200 mg, or about 180 mg of pyridostigmine bromide. In certain embodiments, the core can further comprise from about 20 mg to about 100 mg, from about 40 mg to about 80 mg, or from about 50 mg to about 60 mg of succinic acid. In certain embodiments, the core can further comprise from about 20 mg to about 80 mg, from about 30 mg to about 65 mg, or from about 45 mg to about 55 mg of sodium bicarbonate. In certain embodiments, the core can further comprise from about 40 mg to about 200 mg, from about 50 mg to about 150 mg, or from about 60 mg to about 130 mg of calcium carbonate. In certain embodiments, the core can further comprise from about 50 mg to about 200 mg, or about 100 mg of crospovidone. In certain embodiments, the core can further comprise from about 100 mg to about 300 mg, from about 150 mg to about 250 mg, or about 230 mg of mannitol. In certain embodiments, the core can further optionally comprise from about 50 mg to about 350 mg, from about 100 mg to about 300 mg, or about 200 mg of hydroxypropyl methylcellulose with an average molecular weight of about 400,000. In certain embodiments, the core can further optionally comprise from about 150 mg to about 350 mg, or from about 200 mg to about 300 mg of hydroxypropyl methylcellulose with an average molecular weight of about 164,000. In certain embodiments, the core can further comprise from about 1 mg to about 10 mg, from about 2 mg to about 7 mg, or about 4 mg of fumed silica. In certain embodiments, the core can further comprise from about 1 mg to about 15 mg, from about 5 mg to about 10 mg, or about 8 mg of magnesium stearate. In certain embodiments, the pyridostigmine bromide gastroretentive tablets can further comprise a seal coat. In certain embodiments, the seal coat can comprise one or more of triethyl citrate, talc, hydroxypropyl cellulose, and combinations thereof. In certain embodiments, the pyridostigmine bromide gastroretentive tablets can further comprise a functional coat over the seal coat. In certain embodiments, the functional coat of the pyridostigmine bromide gastroretentive tablets can comprise from about 100 mg to about 200 mg, from about 125 mg to about 175 mg, or from about 145 mg to about 150 mg of a copolymer of ethyl acrylate, methyl methacrylate, and trimethylammonioethyl methacrylate chloride (1:2:0.2) (ammonio methacrylate copolymer). In certain embodiments, the functional coat of the pyridostigmine bromide gastroretentive tablets can further comprise from about 10 mg to about 50 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 20 mg to about 40 mg, or from about 25 mg to about 35 mg of talc. In certain embodiments, the pyridostigmine bromide gastroretentive tablets can comprise an over coat. In certain embodiments, the over coat can comprise from about 5 mg to about 30 mg, from about 10 mg to about 20 mg, or about 15 mg of polyvinyl alcohol-based polymer.

In certain embodiments, the pyridostigmine bromide gastroretentive tablets can comprise a core, and a functional coat. In certain embodiment, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, methylcellulose, fumed silica, magnesium stearate, and combinations thereof. In certain embodiments, the core of the pyridostigmine bromide gastroretentive tablets can comprise from about 50 mg to about 200 mg, from about 100 mg to about 150 mg, or about 135 mg of pyridostigmine bromide. In certain embodiments, the core can further comprise from about 40 mg to about 150 mg, from about 60 mg to about 100 mg, or from about 75 mg to about 85 mg of succinic acid. In certain embodiments, the core can further comprise from about 20 mg to about 80 mg, from about 30 mg to about 65 mg, or from about 45 mg to about 55 mg of sodium bicarbonate. In certain embodiments, the core can further comprise from about 10 mg to about 100 mg, from about 25 mg to about 75 mg, or from about 60 mg to 70 mg of calcium carbonate. In certain embodiments, the core can further comprise from about 50 mg to about 200 mg, or about 100 mg of crospovidone. In certain embodiments, the core can further comprise from about 100 mg to about 300 mg, from about 150 mg to about 275 mg, or from about 200 mg to about 255 mg of mannitol. In certain embodiments, the core can further optionally comprise from about 50 mg to about 250 mg, from about 100 mg to about 20 mg, or about 150 mg of hydroxypropyl methylcellulose with an average molecular weight of about 400,000. In certain embodiments, the core can further comprise from about 100 mg to about 450 mg, from about 150 mg to about 350 mg, or from about 150 mg to about 300 mg of hydroxypropyl methylcellulose with an average molecular weight of about 164,000. In certain embodiments, the core can further comprise from about 1 mg to about 10 mg, from about 2 mg to about 8 mg, or from about 3 mg to about 5 mg of fumed silica. In certain embodiments, the core can further comprise from about 1 mg to about 15 mg, from about 5 mg to about 10 mg, or about 8 mg of magnesium stearate. In certain embodiments, the pyridostigmine bromide gastroretentive tablets can further comprise a functional coat. In certain embodiments, the functional coat can comprise ammonio methacrylate copolymer, triethyl citrate, talc, and combinations thereof. In certain embodiments, the functional coat of the pyridostigmine bromide gastroretentive tablets can comprise from about 100 mg to about 250 mg, from about 125 mg to about 200 mg, or from about 145 mg to about 190 mg of ammonio methacrylate copolymer. In certain embodiments, the functional coat of the pyridostigmine bromide gastroretentive tablets can further comprise from about 10 mg to about 50 mg, from about 15 mg to about 35 mg, from about 20 mg to about 30 mg of triethyl citrate. In certain embodiments, the functional coat of the pyridostigmine bromide gastroretentive tablets can further comprise from about 10 mg to about 50 mg, from about 20 mg to about 40 mg, or from about 25 mg to about 30 mg of talc.

In certain embodiments, the pyridostigmine bromide gastroretentive tablets can comprise a core, and a functional coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 50 mg to about 200 mg, from about 100 mg to about 150 mg, or about 135 mg of pyridostigmine bromide. In certain embodiments, the core can further comprise from about 50 mg to about 150 mg, from about 80 mg to about 30 mg, or about 125 mg of succinic acid. In certain embodiments, the core can further comprise from about 30 mg to about 100 mg, or from about 50 mg to about 75 mg of sodium bicarbonate. In certain embodiments, the core can further comprise from about 25 mg to about 150 mg, from about 50 mg to about 100 mg, or from about 60 mg to about 75 mg of calcium carbonate. In certain embodiments, the core can further comprise from about 100 mg to about 300 mg, from about 150 mg to about 250 mg, or about 200 mg of crospovidone. In certain embodiments, the core can further comprise from about 10 mg to about 200 mg, from about 25 mg to about 50 mg, from about 100 mg to about 200 mg, or from about 150 mg to about 175 mg of mannitol. In certain embodiments, the core can further optionally comprise from about 25 mg to about 150 mg, from about 50 mg to about 125 mg, or about 100 mg of hydroxypropyl methylcellulose with an average molecular weight of about 400,000. In certain embodiments, the core can further comprise from about 50 mg to about 450 mg, or from about 100 mg to about 200 mg of hydroxypropyl methylcellulose with an average molecular weight of about 164,000. In certain embodiments, the core can further comprise from about 1 mg to about 10 mg, from about 2 mg to about 8 mg, or from about 3 mg to about

5 mg of fumed silica. In certain embodiments, the core can further comprise from about 1 mg to about 15 mg, from about 5 mg to about 10 mg, or about 8 mg of magnesium stearate. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 250 mg, from about 100 mg to about 200 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 20 mg to about 40 mg, or from about 25 mg to about 35 mg of talc.

In certain embodiments, the pyridostigmine bromide gastroretentive tablets can comprise a core, and a functional coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose fumed silica, magnesium stearate, and combinations thereof. In certain embodiments, the core of the pyridostigmine bromide gastroretentive tablets can comprise from about 100 mg to about 250 mg, from about 150 mg to about 200 mg, or about 180 mg of pyridostigmine bromide. In certain embodiments, the core can further comprise from about 50 mg to about 150 mg, from about 80 mg to about 30 mg, or about 125 mg of succinic acid. In certain embodiments, the core can further comprise from about 30 mg to about 100 mg, or from about 50 mg to about 75 mg of sodium bicarbonate. In certain embodiments, the core can further comprise from about 25 mg to about 150 mg, from about 70 mg to about 125 mg, or about 100 mg of calcium carbonate. In certain embodiments, the core can further comprise from about 100 mg to about 300 mg, from about 150 mg to about 250 mg, or about 200 mg of crospovidone. In certain embodiments, the core can further comprise from about 50 mg to about 200 mg, from about 75 mg to about 150 mg, or from about 100 mg to about 125 mg of mannitol. In certain embodiments, the core can further optionally comprise from about 25 mg to about 150 mg, from about 50 mg to about 125 mg, or about 100 mg of hydroxypropyl methylcellulose with an average molecular weight of about 400,000. In certain embodiments, the core can further comprise from about 50 mg to about 300 mg, or from about 100 mg to about 200 mg of hydroxypropyl methylcellulose with an average molecular weight of about 164,000. In certain embodiments, the core can further comprise from about 1 mg to about 10 mg, from about 2 mg to about 8 mg, or from about 3 mg to about 5 mg of fumed silica. In certain embodiments, the core can further comprise from about 1 mg to about 15 mg, from about 5 mg to about 10 mg, or about 8 mg of magnesium stearate. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 150 mg, or from about 100 mg to about 125 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 20 mg to about 30 mg of talc.

In certain embodiments, the pyridostigmine bromide gastroretentive tablets can comprise a core, and a functional coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 150 mg to about 400 mg, from about 200 mg to about 450 mg, or from about 250 to about 310 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, or from about 50 mg to about 75 mg of sodium bicarbonate. In certain embodiments, the core can further comprise from about 20 mg to about 100 mg, from about 40 mg to about 80 mg, or from about 60 mg to about 75 mg of calcium carbonate. In certain embodiments, the core can further comprise from about 50 mg to about 150 mg, from about 75 mg to about 125 mg, or about 100 mg of crospovidone. In certain embodiments, the core can further comprise from about 25 mg to about 175 mg, from about 50 mg to about 150 mg, or from about 70 mg to about 125 mg of mannitol. In certain embodiments, the core can further optionally comprise from about 50 mg to about 200 mg, from about 100 mg to about 175 mg, or about 150 mg of hydroxypropyl methylcellulose with an average molecular weight of about 400,000. In certain embodiments, the core can further comprise from about 50 mg to about 200 mg, from about 100 mg to about 175 mg, or about 150 mg of hydroxypropyl methylcellulose with an average molecular weight of about 164,000. In certain embodiment, the core can further comprise from about 1 mg to about 20 mg, from about 5 mg to about 15 mg, or about 10 mg of fumed silica. In certain embodiment, the core can further comprise from about 5 mg to about 20 mg, from about 10 mg to about 15 mg, or about 12 mg of magnesium stearate. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc.

In certain embodiments, the pyridostigmine bromide gastroretentive tablets can comprise a core, a functional coat, a seal coat, a drug layer and an over coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, oxide pigment black, and combinations thereof. In certain embodiments, the core of the pyridostigmine bromide gastroretentive tablets can comprise from about 50 mg to about 200 mg, from about 100 mg to about 150 mg, or about 135 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, from about 50 mg to about 75 mg, or about 55 mg of sodium bicarbonate. In certain embodiments, the core can further comprise from about 20 mg to about 100 mg, from about 40 mg to about 80 mg, or from about 60 mg to about 70 mg of calcium carbonate. In certain embodiments, the core can further comprise from about 50 mg to about 150 mg, from about 75 mg to about 125 mg, or about 100 mg of crospovidone. In certain embodiments, the core can further comprise from about 150 mg to about 400 mg, from about 200 mg to about 350 mg, or from about 250 mg to about 300 mg of mannitol. In certain embodiments, the core can further optionally comprise from about 50 mg to about 200 mg, from about 100 mg to about 175 mg, or about 150 mg of hydroxypropyl methylcellulose with an average molecular weight of about 400,000. In certain embodiments, the core can further comprise from about 50 mg to about 200 mg, from about 100 mg to about 175 mg, or about 150 mg of hydroxypropyl methylcellulose with an average molecular weight of about 164,000. In certain embodiments, the core can further comprise from about 1 mg to about 20 mg, from about 5 mg to about 15 mg, or about 10 mg of fumed silica. In certain embodiments, the core can further comprise from about 5 mg to about 20 mg, from about 10 mg to about 15 mg, or about 12 mg of magnesium stearate. In certain embodiments, the core can further optionally comprise from about 5 mg to about 20 mg, or from about 10 mg to about 15 mg, or about 12 mg of oxide pigment black. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc. In certain embodiments, the pyridostigmine bromide gastroretentive tablets can further comprise a seal coat. In certain embodiments the seal coat can comprise from about 1 mg to about 20 mg, from about 5 mg to about 15 mg, or about 10 mg of polyvinyl alcohol-based polymer. In certain embodiments, the pyridostigmine bromide gastroretentive tablets can further comprise a drug layer. In certain embodiments, the drug layer can comprise pyridostigmine bromide, hydroxypropyl cellulose, and combinations thereof. In certain embodiments, the drug layer can comprise from about 10 mg to about 100 mg, from about 25 mg to about 75 mg, or from about 40 mg to about 50 mg of pyridostigmine bromide. In certain embodiments, the drug layer can comprise from about 1 mg to about 20 mg, from about 5 mg to about 15 mg, or from about 8 mg to about 12 mg of hydroxypropyl cellulose. In certain embodiments, the pyridostigmine bromide gastroretentive tablets can further comprise an over coat. In certain embodiments, the over coat can comprise from about 20 mg to about 60 mg, from about 30 mg to about 50 mg, or about 40 mg of polyvinyl alcohol-based polymer.

In certain embodiments, the pyridostigmine bromide gastroretentive tablets can comprise a core, and a functional coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 200 mg to about 400 mg, from about 250 mg to about 350 mg, or from about 285 mg, to about 315 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, or from about 50 mg to about 75 mg of sodium bicarbonate. In certain embodiments, the core can further comprise from about 20 mg to about 100 mg, from about 40 mg to about 80 mg, or from about 60 mg to about 75 mg of calcium carbonate. In certain embodiments, the core can further comprise from about 50 mg to about 150 mg, from about 75 mg to about 125 mg, or about 100 mg of crospovidone. In certain embodiments, the core can further optionally comprise from about 25 mg to about 175 mg, from about 50 mg to about 150 mg, from about 60 mg to about 100 mg, or from about 70 mg to about 85 mg of mannitol. In certain embodiments, the core can further comprise from about 100 mg to about 300 mg, from about 125 mg to about 250 mg, or from about 150 mg to about 200 mg of hydroxypropyl methylcellulose with an average molecular weight of about 400,000. In certain embodiments, the core can further comprise from about 100 mg to about 300 mg, from about 125 mg to about 250 mg, or from about 150 mg to about 200 mg of hydroxypropyl methylcellulose with an average molecular weight of about 164,000. In certain embodiments, the core can further comprise from about 1 mg to about 20 mg, from about 5 mg to about 15 mg, or about 10 mg of fumed silica. In certain embodiments, the core can further comprise from about 5 mg to about 20 mg, from about 10 mg to about 15 mg, or about 12 mg of magnesium stearate. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc.

In certain embodiments, the pyridostigmine bromide gastroretentive tablets can comprise a core, and a functional coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 100 mg to about 300 mg, from about 175 mg to about 275 mg, from about 195 mg to about 210 mg, or from about 225 mg to about 260 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, or from about 50 mg to about 75 mg of sodium bicarbonate. In certain embodiments, the core can further comprise from about 20 mg to about 100 mg, from about 40 mg to about 80 mg, or from about 60 mg to about 75 mg of calcium carbonate. In certain embodiments, the core can further comprise from about 50 mg to about 150 mg, from about 75 mg to about 125 mg, or about 100 mg of crospovidone. In certain embodiments, the core can further optionally comprise from about 50 mg to about 200 mg, from about 100 mg to about 175 mg, from about 120 mg to about 125 mg, or from about 120 mg to about 175 mg of mannitol. In certain embodiments, the core can further comprise from about 100 mg to about 300 mg, from about 125 mg to about 250 mg, or from about 150 mg to about 215 mg of hydroxypropyl methylcellulose with an average molecular weight of about 400,000. In certain embodiments, the core can further comprise from about 100 mg to about 300 mg, from about 125 mg to about 250 mg, or from about 150 mg to about 215 mg of hydroxypropyl methylcellulose with an average molecular weight of about 164,000. In certain embodiments, the core can further comprise from about 1 mg to about 20 mg, from about 5 mg to about 15 mg, or about 10 mg of fumed silica. In certain embodiments, the core can further comprise from about 5 mg to about 20 mg, from about 10 mg to about 15 mg, or about 12 mg of magnesium stearate. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc.

In certain embodiments, the pyridostigmine bromide gastroretentive tablets can comprise a core, and a functional coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 50 mg to about 200 mg, from about 70 mg to about 170 mg, or from about 100 mg to about 160 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, or from about 50 mg to about 75 mg of sodium bicarbonate. In certain embodiments, the core can further comprise from about 20 mg to about 100 mg, from about 40 mg to about 80 mg, or from about 60 mg to about 75 mg of calcium carbonate. In certain embodiments, the core can further comprise from about 50 mg to about 150 mg, from about 75 mg to about 125 mg, or about 100 mg of crospovidone. In certain embodiments, the core can further optionally comprise from about 200 mg to about 350 mg, from about 210 mg to about 310 mg, or from about 220 to about 280 mg of mannitol. In certain embodiments, the core can further comprise from about 100 mg to about 300 mg, from about 125 mg to about 250 mg, or from about 150 mg to about 200 mg of hydroxypropyl methylcellulose with an average molecular weight of about 400,000. In certain embodiments, the core can further comprise from about 100 mg to about 300 mg, from about 125 mg to about 250 mg, or from about 150 mg to about 200 mg of hydroxypropyl methylcellulose with an average molecular weight of about 164,000. In certain embodiments, the core can further comprise from about 1 mg to about 20 mg, from about 5 mg to about 15 mg, or about 10 mg of fumed silica. In certain embodiments, the core can further comprise from about 5 mg to about 20 mg, from about 10 mg to about 15 mg, or about 12 mg of magnesium stearate. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc.

In certain embodiments, the pyridostigmine bromide gastroretentive tablets can comprise a core, a functional coat, a seal coat, a drug layer, and an over coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 50 mg, to about 200 mg, from about 100 mg to about 150 mg, or about 135 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, from about 50 mg to about 75 mg, or about 55 mg of sodium bicarbonate. In certain embodiments, the core can further comprise from about 20 mg to about 100 mg, from about 40 mg to about 80 mg, or from about 60 mg to about 70 mg of calcium carbonate. In certain embodiments, the core can further comprise from about 50 mg to about 150 mg, from about 75 mg to about 125 mg, or about 100 mg of crospovidone. In certain embodiments, the core can further comprise from about 175 mg to about 300 mg, from about 200 mg to about 275 mg, or from about 230 mg to 240 mg of mannitol. In certain embodiments, the core can further optionally comprise from about 50 mg to about 200 mg, from about 100 mg to about 175 mg, or about 150 mg of hydroxypropyl methylcellulose with an average molecular weight of about 400,000. In certain embodiments, the core can further comprise from about 50 mg to about 200 mg, from about 100 mg to about 175 mg, or about 150 mg of hydroxypropyl methylcellulose with an average molecular weight of about 164,000. In certain embodiments, the core can further comprise from about 1 mg to about 20 mg, from about 5 mg to about 15 mg, or about 10 mg of fumed silica. In certain embodiments, the core can further comprise from about 5 mg to about 20 mg, from about 10 mg to about 15 mg, or about 12 mg of magnesium stearate. In certain embodiments, the core can further comprise from about 1 mg to about 20 mg, from about 5 mg to about 15 mg, or from about 7 mg to about 12 mg of oxide pigment black. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc. In certain embodiments, the pyridostigmine bromide gastroretentive tablets can further comprise a seal coat. In certain embodiments, the seal coat can comprise polyvinyl alcohol-based polymer. In certain embodiments, the seal coat can comprise from about 1 mg to about 20 mg, from about 5 mg to about 15 mg, or about 10 mg of polyvinyl alcohol-based polymer. In certain embodiments, the pyridostigmine bromide gastroretentive tablets can further comprise a drug layer. In certain embodiments, the drug layer can comprise pyridostigmine bromide, and hydroxypropyl cellulose. In certain embodiments, the drug layer can comprise from about 20 mg to about 75 mg, from about 30 mg to about 60 mg, or from about 40 mg to about 50 mg of pyridostigmine bromide. In certain embodiments, the drug layer can further comprise from about 1 mg to about 20 mg, from about 5 mg to about 15 mg, or about 9 mg of hydroxypropyl cellulose. In certain embodiments, the pyridostigmine bromide gastroretentive tablets can further comprise an over coat. In certain embodiments, the overcoat can comprise polyvinyl alcohol-based polymer. In certain embodiments, the over coat can comprise from about 10 mg to about 60 mg, from about 20 mg to about 50 mg, or about 40 mg of polyvinyl alcohol-based polymer.

In certain embodiments, the pyridostigmine bromide gastroretentive tablets can comprise a core, a functional coat, a seal coat, a drug layer, and an over coat. In certain embodiments, the core of the pyridostigmine bromide gastroretentive tablets can comprise one or more of pyridostigmine bromide, succinic acid, sodium bicarbonate, calcium carbonate, crospovidone, mannitol, hydroxypropyl methylcellulose, fumed silica, magnesium stearate, and combinations thereof. In certain embodiments, the core of the pyridostigmine bromide gastroretentive tablets can comprise from about 25 mg to about 125 mg, from about 50 mg to about 100 mg, or about 75 mg of pyridostigmine bromide. In certain embodiments, the core can further comprise from about 25 mg to about 125 mg, from about 50 mg to about 100 mg, or from about 75 mg to about 90 mg of succinic acid. In certain embodiments, the core can further comprise from about 30 mg to about 100 mg, from about 50 mg to about 75 mg, or about 55 mg of sodium bicarbonate. In certain embodiments, the core can further comprise from about 20 mg to about 100 mg, from about 40 mg to about 80 mg, or from about 60 mg to about 70 mg of calcium carbonate. In certain embodiments, the core can further comprise from about 50 mg to about 150 mg, from about 75 mg to about 125 mg, or about 100 mg of crospovidone. In certain embodiments, the core can further comprise from about 200 mg to about 350 mg, from about 250 mg to about 300 mg, or from about 270 mg to about 280 mg of mannitol. In certain embodiments, the core can further optionally comprise from about 50 mg to about 200 mg, from about 100 mg to about 175 mg, or about 150 mg of hydroxypropyl methylcellulose with an average molecular weight of about 400,000. In certain embodiments, the core can further comprise from about 50 mg to about 200 mg, from about 100 mg to about 175 mg, or about 150 mg of hydroxypropyl methylcellulose with an average molecular weight of about 164,000. In certain embodiments, the core can further comprise from about 1 mg to about 20 mg, from about 5 mg to about 15 mg, or about 10 mg of fumed silica. In certain embodiments, the core can further comprise from about 5 mg to about 20 mg, from about 10 mg to about 15 mg, or about 12 mg of magnesium stearate. In certain embodiments, the core can further comprise from about 1 mg to about 20 mg, from about 5 mg to about 15 mg, or from about 7 mg to about 12 mg of oxide pigment black. In certain embodiments, the pyridostigmine bromide gastroretentive tablets can further comprise a functional coat. In certain embodiments, the functional coat can comprise one or more of ammonio methacrylate copolymer, triethyl citrate, talc, and combinations thereof. In certain embodiments, the functional coat of the pyridostigmine bromide gastroretentive tablets can comprise from about 50 mg to about 200 mg, from about 75 mg to about 175 mg, or from about 125 mg to about 150 mg of ammonio methacrylate copolymer. In certain embodiments, the functional coat of the pyridostigmine bromide gastroretentive tablets can further comprise from about 10 mg to about 40 mg, from about 15 mg to about 30 mg, or from about 20 mg to about 25 mg of triethyl citrate. In certain embodiments, the functional coat of the pyridostigmine bromide gastroretentive tablets can further comprise from about 10 mg to about 50 mg, from about 15 mg to about 40 mg, or from about 25 mg to about 30 mg of talc. In certain embodiments, the pyridostigmine bromide gastroretentive tablets can further comprise a seal coat. In certain embodiments, the seal coat can comprise polyvinyl alcohol-based polymer. In certain embodiments, the seal coat can comprise from about 1 mg to about 20 mg, from about 5 mg to about 15 mg, or about 10 mg of polyvinyl alcohol-based polymer. In certain embodiments, the pyridostigmine bromide gastroretentive tablets can further comprise a drug layer. In certain embodiments, the drug layer can comprise one of more of pyridostigmine bromide, and hydroxypropyl cellulose and combinations thereof. In certain embodiments, the drug layer can comprise from about 10 mg to about 50 mg, from about 20 mg to about 40 mg, or about 30 mg of pyridostigmine bromide. In certain embodiments, the drug layer can further comprise from about 1 mg to about 20 mg, from about 5 mg to about 15 mg, or about 9 mg of hydroxypropyl cellulose. In certain embodiments, the pyridostigmine bromide gastroretentive tablets can further comprise an over coat. In certain embodiments, the overcoat can comprise polyvinyl alcohol-based polymer. In certain embodiments, the over coat can comprise from about 10 mg to about 60 mg, from about 20 mg to about 50 mg, or about 40 mg of polyvinyl alcohol-based polymer.

### 6.2.2.6 Features of Gastroretentive Dosage Forms

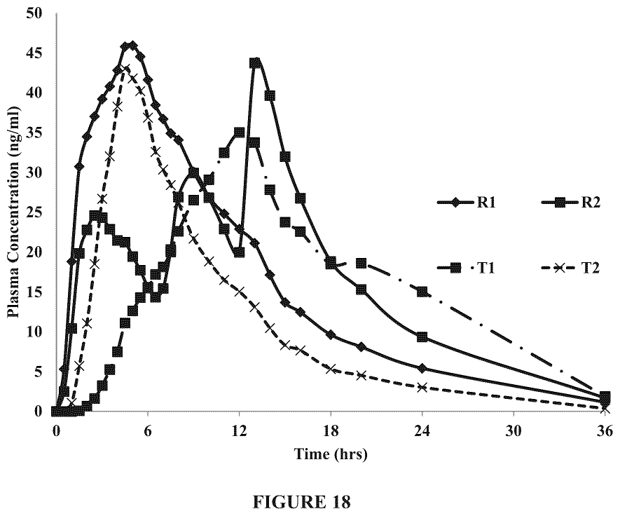

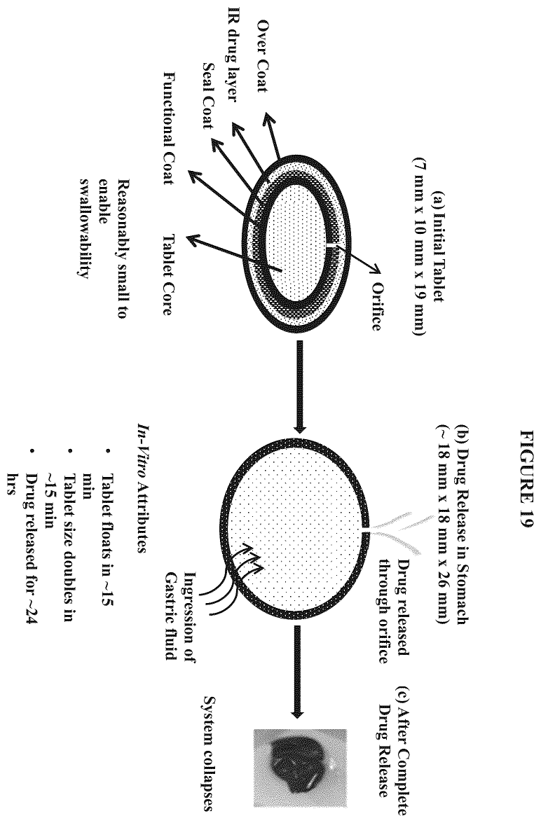

The gastroretentive tablets of the disclosure combine the following two key attributes: gastric retention and continuous controlled drug delivery in therapeutic range (e.g, from about 15 ng/ml to about 50 ng/ml) for up to about 24 hours. In certain embodiments, the disclosure provides gastroretentive tablets of pyridostigmine or a pharmaceutically acceptable salt thereof that are suitable for providing therapeutic pyridostigmine levels, with reduced initial burst release of the drug, for an extended period of time. In certain embodiments, the disclosure provides gastroretentive tablets of pyridostigmine or a pharmaceutically acceptable salt thereof that are suitable for providing stable pyridostigmine levels by lowering Fluctuation Index, and Cmax, compared to marketed pyridostigmine products, e.g., MESTINON TIMESPAN® and MESTINON. This is particularly desirable for myasthenia gravis (MG) patients, as a constant therapeutic level of pyridostigmine has been shown to improve therapeutic outcome and quality of life. Quality of life and compliance are also enhanced with the reduction or elimination of initial burst release of pyridostigmine, as experienced with the currently marketed ER formulation, and the concomitant reduction in undesirable side effects. FIG. 18 compares pharmacokinetic data for gastroretentive Tablet 8 (T₁), pellet compositions T₂, and marketed pyridostigmine products, e.g., MESTINON® tablets (60 mg/TID) (R₂) and MESTINON® TIMESPAN® tablets (180 mg/QD) (R₁). FIG. 18 demonstrates that bioavailability of gastroretentive Tablet 8 (T₁) is comparable to MESTINON® (R₂) and MESTINON® TIMESPAN® tablets (R₁) in the fed state. Pharmacokinetic data from FIG. 18 demonstrates that gastroretentive tablets of the disclosure (T1), by releasing the drug in the upper GI tract, provide comparable bioavailability to marketed pyridostigmine products (R1), and provide extended plasma concentration profiles for 24 hours. In certain embodiments, the gastroretentive tablets of the present disclosure provide extended release, with reduced initial burst release, of pyridostigmine or a pharmaceutically acceptable salt thereof of at least 14 hours. In particular embodiments, the reduced initial burst release comprises an in vitro release of less than about 35 wt % of the pyridostigmine or a pharmaceutically acceptable salt thereof, based on the total weight of the pyridostigmine bromide present in the tablet, within 2 hours of dissolution in a dissolution medium.

The gastroretentive pyridostigmine tablets of the disclosure provide significant therapeutic advantages over the currently marketed pyridostigmine products with respect to the following attributes: 1) enhanced control of symptoms associated with MG with once-a-day dosing, 2) rapid onset of effect/reduced lag time and consistent blood levels in therapeutic range during the daytime to treat progressive muscle weakness and fatigue known to build up by the evening, 3) reduced initial drug concentration (e.g., reduced or minimized initial burst release) sufficient to provide therapeutic effect without GI side effects, 4) lower, but still therapeutic, blood levels during the night for treating nighttime symptoms and providing uninterrupted sleep, 5) improved tolerability of the drug with reduced adverse events compared to fluctuating blood levels from an IR formulation, 6) reduced pill-burden and improved adherence/compliance due to less frequent dosing and reduced pill-burden, and 7) better patient satisfaction and quality of life with improved functionality throughout the day and reduced reliance on caregivers.

In certain embodiments, the gastroretentive pyridostigmine compositions of the disclosure improve therapeutic outcome by reducing “wearing off,” effects (lower trough levels between dosing may result in inconsistent symptom control and gaps in efficacy due to subtherapeutic levels). In certain embodiments, the gastroretentive pyridostigmine compositions of the disclosure reduce low trough levels (increase Cmin) between consecutive doses and prevent worsening of symptoms, thereby improving quality of life. In certain embodiments, the gastroretentive pyridostigmine compositions of the disclosure reduce Fluctuation Index and Cmax:Cmin ratio compared to the marketed pyridostigmine products, e.g., MESTINON and MESTINON TIMESPAN®. Example 19/Table 23 compares FI of MESTINON® and Tablet 37 under fed conditions (e.g., MF-MC condition and HF-HC condition), from a single dose and multiple dose cross-over pharmacokinetic (PK) study conducted in healthy volunteers under fed conditions (MF-MC and HF-HC conditions) to evaluate and compare PK performance of extended release compositions of the disclosure using Tablet 37, administered once-a-day, and marketed pyridostigmine bromide tablet, MESTINON® (60 mg), administered thrice-a-day. Table 23 demonstrates that Tablet 37 exhibits a lower FI, under MF-MC and HF-HC conditions, compared to the marketed pyridostigmine product, e.g., MESTINON® (60 mg×3).

In certain embodiments, a decrease in Fluctuation Index indicates a decrease in drug plasma fluctuation between the peak-to-trough plasma levels (e.g., Cmax:Cmin ratio) of pyridostigmine. In certain embodiments, the gastroretentive extended release pyridostigmine compositions of the disclosure, upon a single dose administration, provide a Fluctuation Index from about 0.1 to about 1. In certain embodiments, the gastroretentive extended release pyridostigmine compositions of the disclosure provide a Fluctuation Index (Cmax-Cmin/Cav) of about 0.1, about 0.2, about 0.3, about 0.4, about 0.5, about 0.6, about 0.7, about 0.8, about 0.9, about 1, or any intermediate values therein.

In certain embodiments, the gastroretentive extended release pyridostigmine compositions of the disclosure release less than 35% of pyridostigmine or a pharmaceutically acceptable salt thereof during first hour of oral ingestion. In certain embodiments, the gastroretentive extended release compositions of the disclosure avoid unwanted GI side effects, generally experienced with the currently marketed pyridostigmine products, e.g., MESTINON and MESTINON TIMESPAN®, by releasing less than 35% (also referred to herein as “reduced initial burst release”) of pyridostigmine or a pharmaceutically acceptable salt thereof during first hour of oral ingestion, providing consistent blood levels of pyridostigmine or a pharmaceutically acceptable salt thereof in a therapeutic range, reducing Fluctuation Index, lowering Cmax, and increasing Cmin. The gastroretentive extended release dosage forms of the disclosure, with or without the IR drug layer, minimize the GI side effects, and provide and maintain therapeutic plasma concentrations of pyridostigmine or a pharmaceutically acceptable salt thereof for a period of at least about 8 hours. In certain embodiments, therapeutic plasma concentration of pyridostigmine or a pharmaceutically acceptable salt thereof is from about 15 ng/ml to about 50 ng/ml. In certain embodiments, therapeutic plasma concentration of pyridostigmine or a pharmaceutically acceptable salt thereof is about 15 ng/ml, about 16 ng/ml, about 17 ng/ml, about 18 ng/ml, about 19 ng/ml, about 20 ng·ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, or any intermediate values therein.

In certain embodiments, the gastroretentive extended release pyridostigmine compositions of the disclosure provide 24-hour symptom control, tolerability, and reduced pill-burden by maintaining therapeutic plasma concentration of pyridostigmine or a pharmaceutically acceptable salt thereof over the 24-hour dosing period. In certain embodiments, the extended release pyridostigmine compositions of the disclosure provide 24-hour symptom control, e.g., providing nighttime and early morning functionality, by maintaining therapeutic plasma levels during the 24-hour dosing period. In certain embodiments, the gastroretentive extended release pyridostigmine compositions of the disclosure provide residual plasma levels of the drug in the morning, such that patients wake up feeling more refreshed and more functional before taking the morning dose, as compared with the currently marketed pyridostigmine products.

In certain embodiments, the gastroretentive extended release pyridostigmine compositions of the disclosure provide lower Fluctuation Index compared to marketed pyridostigmine products, e.g., MESTINON and MESTINON TIMESPAN®, to provide a 24-hour release profile with superior all day coverage and improved tolerability compared to the marketed pyridostigmine products. In certain embodiments, the gastroretentive dosage forms of the present disclosure, upon single dose administration, provide extended release of pyridostigmine bromide, with a Fluctuation Index of from about 0.1 to about 1, for at least about 8 hours, e.g., for up to about 24 hours.

In certain embodiments, the gastroretentive extended release pyridostigmine compositions of the disclosure, under fed conditions, provide higher Cmin/trough level compared to the marketed pyridostigmine products, e.g., MESTINON and MESTINON TIMESPAN®. In certain embodiments, the Cmin, under fed conditions is at least about 15 ng/ml. In certain embodiments, Cmin, under fed conditions, is from 20 ng/ml to 30 ng/ml. In certain embodiments, Cmin under fed conditions is about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, or any intermediate values therein. In certain embodiments, the gastroretentive extended release pyridostigmine compositions of the disclosure reduce worsening of symptoms and improve quality of life by increasing the Cmin/trough levels. In certain embodiments, higher Cmin/trough level of extended release pyridostigmine compositions of the disclosure, compared to the marketed pyridostigmine products, e.g., MESTINON and MESTINON TIMESPAN®, reduce wearing off effects and worsening of symptoms, e.g., provide a better 24-hour control of Myasthenia Gravis symptoms.

In certain embodiments, the gastroretentive extended release pyridostigmine compositions of the disclosure provide lower Cmax/blunted Cmax compared to the marketed pyridostigmine products, e.g., MESTINON and MESTINON TIMESPAN®. In certain embodiments, the Cmax, under fed conditions is less than 60 ng/ml. In certain embodiments, Cmax, under fed conditions, is from 20 ng/ml to 50 ng/ml. In certain embodiments, the Cmax, under fed conditions is about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, or any intermediate values therein. In certain embodiments, the extended release pyridostigmine compositions of the disclosure improve tolerability and reduce side effects by reducing the Cmax and Fluctuation Index. In certain embodiments, lower Cmax of extended release pyridostigmine compositions of the disclosure, compared to the marketed pyridostigmine products, e.g., MESTINON and MESTINON TIMESPAN®, reduce cholinergic side effects, e.g., muscle cramps, and provide better 24-hour control of Myasthenia Gravis symptoms. In certain embodiments, the gastroretentive pyridostigmine compositions of the disclosure provide a Cmax:Cmin ratio of from 1 to 2. In certain embodiments, the gastroretentive pyridostigmine compositions of the disclosure provide a Cmax:Cmin ratio of about 1, about 1.1, about 1.2, about 1.3, about 1.4, about 1.5, about 1.6, about 1.7, about 1.8, about 1.9, about 2, or any intermediate values therein.

In certain embodiments, the gastroretentive extended release pyridostigmine compositions of the disclosure reduce pill-burden compared to the approved pyridostigmine compositions, e.g., MESTINON® and MESTINON® TIMESPAN®. The extended release compositions of pyridostigmine compositions of the disclosure are suitable for once-daily dosing and significantly reduce pill-burden compared to marketed pyridostigmine products, e.g., MESTINON® and MESTINON® TIMESPAN®.

In certain embodiments, the gastroretentive compositions of the disclosure can comprise an immediate release component and an extended release component. The immediate release component comprises immediate release drug layer and the extended release component comprises a core coated with a permeable elastic membrane/functional coat containing at least one orifice. The immediate release drug layer comprises pyridostigmine or a pharmaceutically acceptable salt thereof. The core comprises pyridostigmine bromide, at least one gas-generating agent (e.g., sodium bicarbonate, calcium carbonate), at least one acid (e.g., succinic acid), and at least one swellable water-soluble hydrophilic polymer. The permeable elastic membrane comprises at least one copolymer of ethyl acrylate, methyl methacrylate, and trimethylammonioethyl methacrylate chloride, and at least one plasticizer.

In certain embodiments, amounts of acid(s) and gas-generating agent are optimized to minimize the floating lag time.

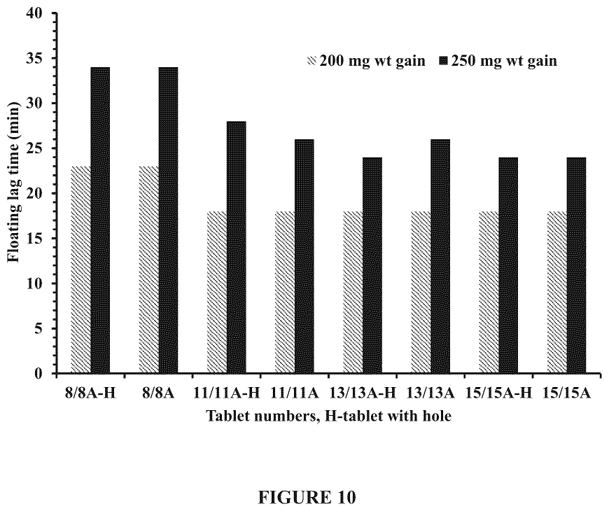

In certain embodiments, compositions of the disclosure include a seal coat between the core and the functional coat. In certain other embodiments, the compositions of the disclosure do not include a seal coat between the core and the functional coat. In certain embodiments, absence of seal coat between the tablet core and the functional coat reduced floating lag time. Tablets 8 and 8A contained a seal coat between the tablet core and the functional coat; and Tablets 11/11A, 13/13A, and 15/15A did not include a seal coat between the tablet core and the functional coat. FIG. 10 compares floating lag times of Tablets 8, 11, 13, and 15, with and without orifice/hole, at 200 mg functional coating weight gain, and Tablets 8A, 11A, 13A, and 15A, with and without orifice/hole, at 250 mg functional coating weight gain. The flotation studies were performed, using rotating bottle method at 5 rpm and 37° C., in 200 ml of 50 mM pH 4.5 acetate buffer containing 100 mM NaCl. FIG. 10 demonstrates that Tablets 8/8A, containing a seal coat, exhibit longer floating lag times compared to tablets without a seal coat (Tablets 11/11A, 13/13A, and 15/15A).

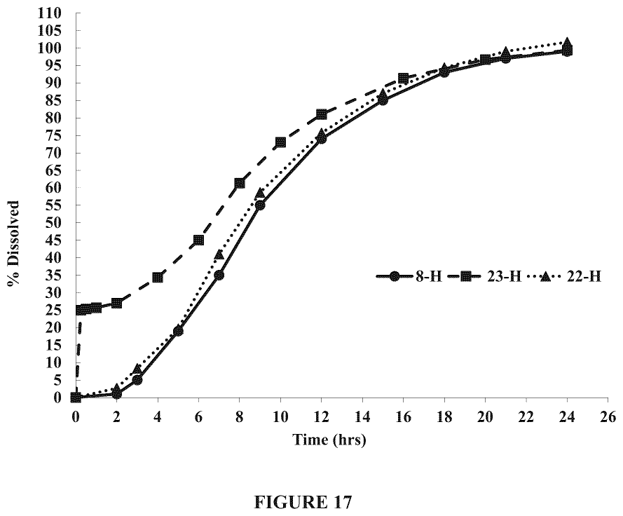

In certain embodiments, tablets containing a mixture of BENECEL™ K4M PH DC (2700-5040 mPa·s) and METHOCEL™ K100 Prem LVCR (80-120 mPa·s) (Tablets 22, 23, and 34) provided a better controlled release compared to tablets containing BENECEL™® K4M PH DC (Tablet 8) alone. FIGS. 17 and 23 compare in vitro dissolution profiles of Tablets 8, 22, and 23; and Tablets 8 and 34, respectively. FIGS. 17 and 23 demonstrate that Tablets 22, 23, and 34 (all containing a mixture of BENECEL™ K4M PH DC and METHOCEL™ K100 Prem LVCR/LVDC2) provided better controlled release compared to Tablet 8 (containing BENECEL™ K4M PH DC alone).

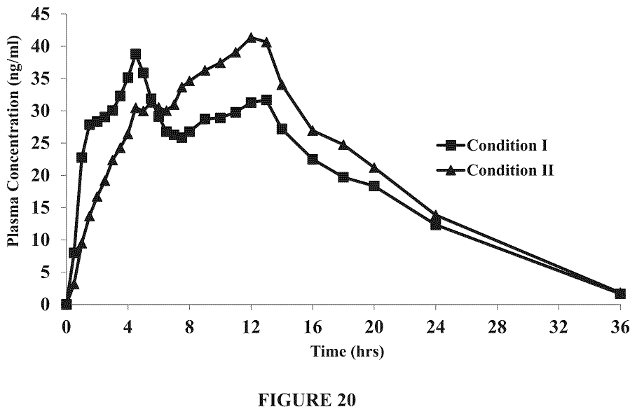

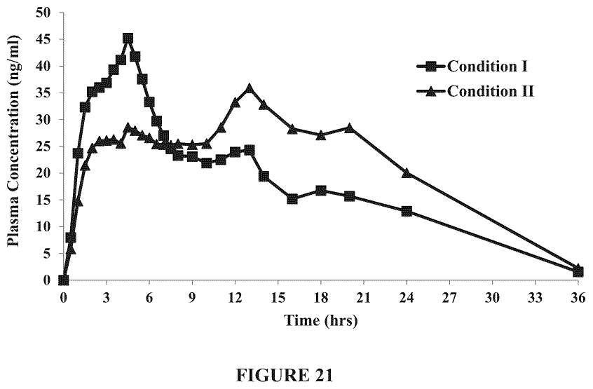

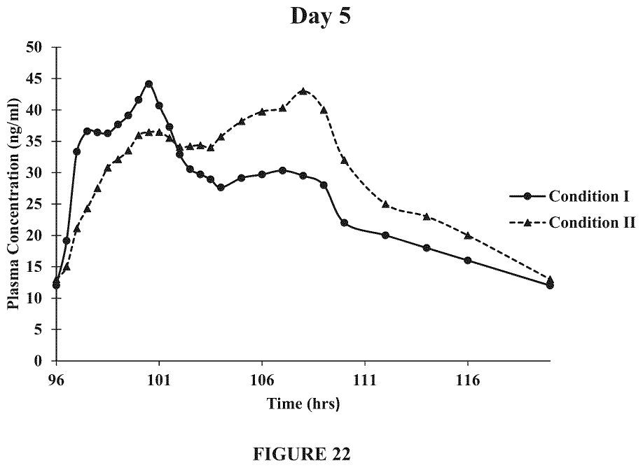

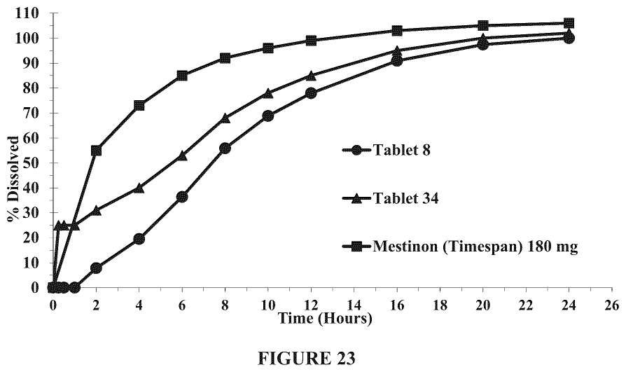

FIGS. 20 and 21 compare pharmacokinetic data for gastroretentive Tablet 34 (gastroretentive tablet with hole) and Tablet 35 (gastroretentive tablet without hole), respectively. FIGS. 20 and 21 demonstrate that Tablets 34 and 35 provide therapeutic plasma concentrations of pyridostigmine bromide from about 14 hours to about 20 hours during a single 24 hour dosing period.

In certain embodiments, the gastroretentive tablets of the disclosure can include an immediate release component layer and an extended release component. The immediate release component comprises an immediate release drug layer. FIG. 23 compares in vitro dissolution profiles of a tablet containing an immediate release drug layer (Tablet 34), a tablet with no immediate release drug layer (Tablet 8), and a tablet of MESTINON® TIMESPAN®. The figure demonstrates that Tablet 8 (without IR drug layer) exhibits minimized initial burst release; and Tablet 34 (with IR drug layer) provides reduced initial burst release (less than about 35% drug release in about 2 hours) of the drug.

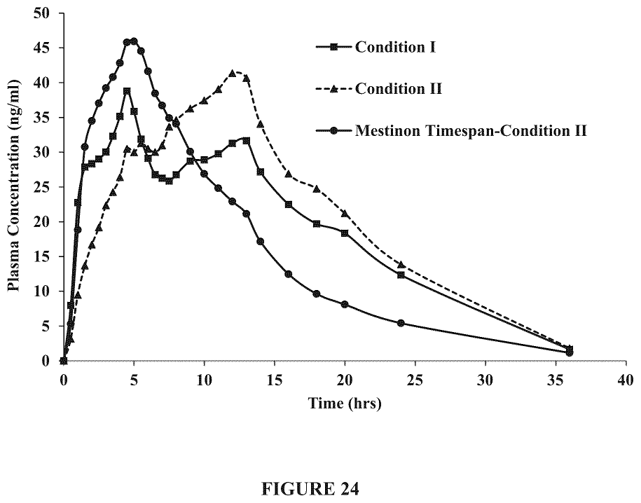

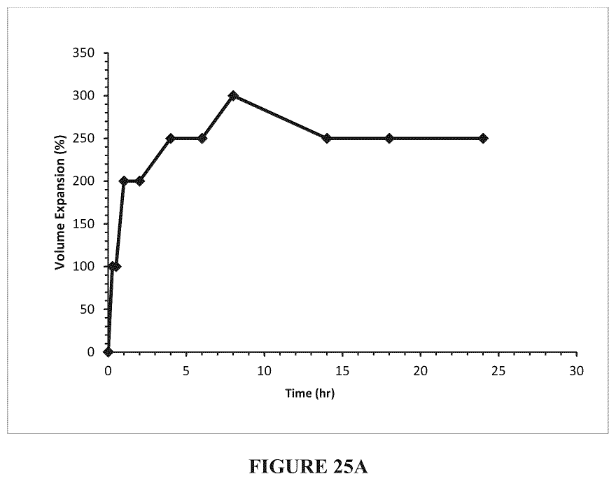

FIG. 24 compares pharmacokinetic data for gastroretentive Tablet 34, a tablet with a hole in the functional coat, under LF-LC breakfast conditions (Condition I) and HF-HC breakfast conditions (Condition II), and MESTINON® TIMESPAN® (Condition II). FIG. 24 demonstrates that MESTINON® TIMESPAN® provides higher drug plasma concentrations between about 0 and 5 hours compared to Tablet 34 under Conditions I and II. FIG. 24 further demonstrates that Tablet 34, under Conditions I and II, provides higher drug plasma concentrations of pyridostigmine bromide over an extended time period, e.g., about 7 hours or beyond, compared to MESTINON® TIMESPAN®.

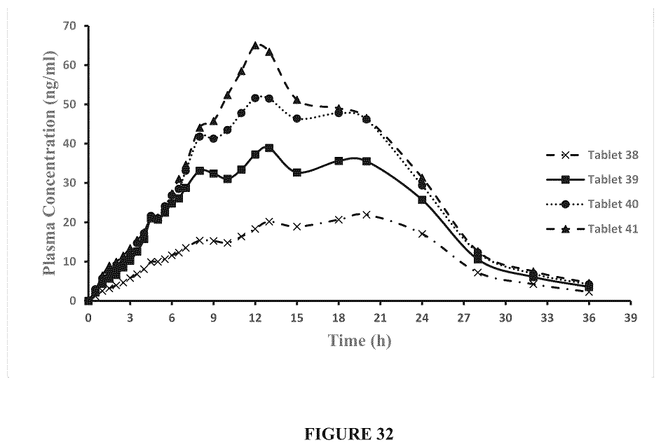

FIG. 32 provides pharmacokinetic data for an open-label, non-randomized, four dose levels, four period, single ascending dose (SAD) study, conducted in 14 healthy, adult human subjects under fed (high fat and caloric content was defined as each meal containing (800-900 Kcal with ˜fat 50%) conditions, to evaluate dose proportionality of the proposed pyridostigmine bromide tablets, 105 mg, 205 mg, 275 mg and 340 mg (Tablets 38-41). Data from FIG. 32/Table 37 shows that estimate of slope for Cₘₐₓ is almost near 1 (e.g., 0.9216), and 90% and 95% Confidence Interval for the slope include 1. Hence, dose proportionality across all strengths (105 mg, 205 mg, 275 mg, and 340 mg) is concluded for Cₘₐₓ.

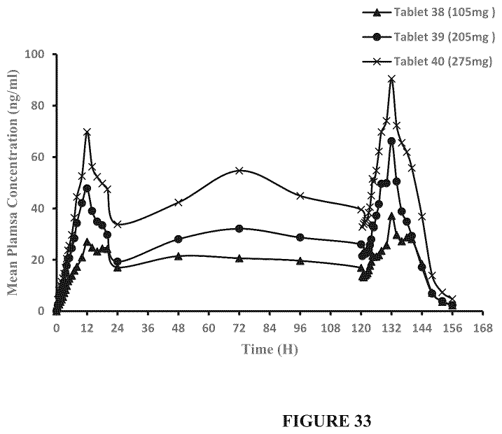

FIG. 33 provides pharmacokinetic data for an open label, non-randomized, four treatment, four cohort, single period, sequential multiple ascending dose study to characterize the pharmacokinetics, safety and tolerability of pyridostigmine after once-daily administration of pyridostigmine bromide Tablet 38 (105 mg), Tablet 39 (205 mg), Tablet 40 (275 mg) and Tablet 41 (340 mg), for six consecutive days in healthy, adult, human subjects under fed conditions [high fat and caloric content was defined as each meal containing (800-900 Kcal with ˜fat 50%)].

In certain embodiments, the compositions of the disclosure can comprise horizontally compressed oval, modified oval, or capsule shapes for easy swallowing. In certain embodiments, the tablets can be compressed using oval, modified oval, capsule shaped, or any other shaping tool. In certain embodiments, the horizontally compressed tablets can comprise a long axis having a length of between about 12 mm and about 22 mm, and a short axis having a length of between about 8 mm and about 11 mm. In certain embodiments, the tablets can have a long axis of about 12 mm, about 13 mm, about 14 mm, about 15 mm, about 16 mm, about 17 mm, about 18 mm, about 19 mm, about 20 mm, about 21 mm, about 22 mm, or any intermediate lengths therein. In certain embodiments, the tablets can have a short axis of about 8 mm, about 9 mm, about 10 mm, about 11 mm, or any intermediate lengths therein. In certain embodiments, the compressed multilayered tablets can comprise a long axis having a length of about 20±2 mm, and a short axis having a length of between about 10±2 mm. In certain embodiments, the initial tablet size (10 mm×19 mm) can be reasonably small for swallowability, and once swallowed, the tablet is designed for rapid generation of carbon dioxide (CO₂) within the core to increase its buoyancy. Within 30 minutes of coming into contact with simulated gastric medium, the tablet starts floating and transforms into an oblong shape with major and minor axes having lengths of about 26 and 18 mm respectively, which can be maintained for at least about 14 hours.

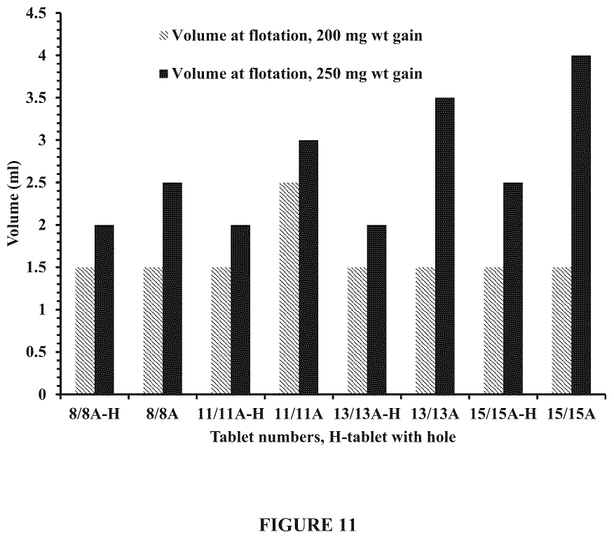

The gastroretentive tablets of the disclosure can comprise an expanding hydrophilic core and a permeable elastic membrane surrounding the core. The membrane can expand rapidly and provide a protective shell that stretches upon hydration to accommodate the rapidly expanding hydrophilic core, and control drug release rate. In certain embodiments, the gastroretentive tablets of the disclosure, when in contact with simulated gastric medium, can expand in about 60 minutes or less, to a size that is equivalent to a size that can prevent the passage of the tablet through the pyloric sphincter. In certain embodiments, the gastroretentive tablets of the disclosure, when in contact with gastric fluid, can float in about 40 minutes or less, expand in about 60 minutes or less to a size that prevents its passage through pyloric sphincter, and provide extended release of pyridostigmine or a pharmaceutically acceptable salt for about 24 hours. In certain embodiments, the gastroretentive tablets of the disclosure can float in about 40 minutes or less in 200 ml of 50 mM pH 4.5 acetate buffer containing 100 mM NaCl, measured using rotating bottle method at 5 rpm and 37° C. In certain embodiments, the gastroretentive tablets of the disclosure can float in about 35 minutes or less, about 34 minutes or less, about 33 minutes or less, about 32 minutes or less, about 31 minutes or less, about 30 minutes or less, about 29 minutes or less, about 28 minutes or less, about 27 minutes or less, about 26 minutes or less, about 25 minutes or less, about 24 minutes or less, about 23 minutes or less, about 22 minutes or less, about 21 minutes or less, about 20 minutes or less, about 19 minutes, or less about 18 minutes or less, about 17 minutes or less, about 16 minutes or less, about 15 minutes or less, or any intermediate time periods, in 200 ml of 50 mM pH 4.5 buffer containing 100 mM NaCl, measured using rotating bottle method at 5 rpm and 37° C. FIG. 10 provides flotation lag times of the compositions of the disclosure containing 200 mg and 250 mg coating weight gains of the functional coat. The compositions provide floating lag time of about 40 minutes or less, measured using rotating bottle method at 5 rpm and 37° C., in 200 ml of 50 mM pH 4.5 acetate buffer containing 100 mM NaCl. In certain embodiments, the gastroretentive tablets of the disclosure can expand in less than about 60 minutes to a size that is large enough to prevent their passage through the pyloric sphincter of a human subject. In certain embodiments, the gastroretentive tablets of the disclosure can exhibit from about 200% to about 800% volume gain from its original volume at 60 minutes, measured using rotating bottle method at about 5 rpm and about 37° C., in 200 ml of pH 4.5 dissolution medium, containing about 100 mM NaCl. In certain embodiments, the gastroretentive tablets can exhibit about 200%, about 250%, about 300%, about 350%, about 400%, about 450%, about 500%, about 550%, about 600%, about 650%, about 700%, about 750%, about 800%, or any intermediate values therein, volume gain from its original volume at 60 minutes FIGS. 11-13 show volume expansions of the gastroretentive tablets of the disclosure, in pH 4.5 buffer containing about 100 mM NaCl. In certain embodiments, rapid expansion of the gastroretentive tablet can result from an initial expansion of the permeable elastic membrane and a simultaneous swelling of the hydrophilic core to support the expanded membrane.

In certain embodiments, the hydrophilic core can swell to a size that can support the expanded permeable elastic membrane. In certain embodiments, the permeable elastic membrane can keep the core intact in a swollen condition for a sufficient period of time and provides the desired characteristics of drug release.

In certain embodiments, the gastroretentive tablets of the disclosure markedly improve absorption and bioavailability of pyridostigmine or a pharmaceutically acceptable salt.

In certain embodiments, the gastroretentive tablets of the disclosure can provide gastric retention of pyridostigmine or a pharmaceutically acceptable salt thereof for up to about 24 hours. In certain embodiments, the gastroretentive tablets of the disclosure can provide gastric retention for about 10 to about 24 hours, about 12 to about 24 hours, and about 14 to about 24 hours. In certain embodiments, the gastroretentive tablets of the disclosure can provide gastric retention for at least about 14 hours. In certain embodiments, the gastroretentive tablets of the disclosure can maintain its integrity in a swollen state for a period of at least about 14 hours. In certain embodiments, the gastroretentive tablets of the disclosure can provide gastric retention for about 24 hours. Furthermore, as the drug diffuses out of the core and the polymeric excipients in the core continue to swell, the plasticizer can leach out and the permeable elastic membrane can lose its integrity and starts to break, thereby allowing remnants of the drug formulation and the remaining contents to be expelled from the stomach at an appropriate time, e.g., after a prolonged period of drug release. FIG. 19 provides schematic and photographic representations of the gastroretentive tablets of the disclosure from its initial tablet form to its residue after complete drug release.

### 6.2.3. Pellets

In certain embodiments, compositions of the disclosure can be formulated as granules or pellets. In certain embodiments, the compositions of the disclosure can be formulated as pyridostigmine bromide pellets. In certain embodiments, the pellets can comprise a pyridostigmine bromide containing core coated with a functional coat/membrane. In certain embodiments, the pyridostigmine bromide containing core can be further drug-layered with a pyridostigmine bromide layer.

In certain embodiments, there can be a seal coat between the pyridostigmine bromide containing core and the functional coat/membrane, and/or between the pyridostigmine bromide layer and the functional coat/membrane. In certain embodiments, the functional coat can be further coated with an immediate release drug layer comprising pyridostigmine bromide. In certain embodiments, the immediate release drug layer is further coated with an overcoat. In certain embodiments, there is a seal coat between the immediate release drug layer and the functional coat, and/or between the immediate release drug layer and the overcoat.

In certain embodiments, the pellets can comprise a microcrystalline cellulose core (MCC), also known as a cellet. In certain embodiments, the MCC core or cellet is drug-layered with a pyridostigmine bromide layer. In certain embodiments, the drug layer can be further coated with a functional coat. In certain embodiments, there can be a seal coat between the drug layer and the functional coat.

In certain embodiments, the drug layer over the pyridostigmine containing core or the cellet core can comprise pyridostigmine bromide, a water-insoluble polymer, a plasticizer, and/or an anti-tacking agent.

In certain embodiments, the water-insoluble polymer can be ethyl cellulose.

In certain embodiments, the anti-tacking agent can be one or more of silicon dioxide (SYLOID® 244FP), fumed silica (CAB-O-SIL®), talc, kaolin, or combinations thereof. In certain embodiments, the plasticizer comprises triethyl citrate, triacetin, polyethylene glycol, propylene glycol, dibutyl sebacate, or combinations thereof. In certain embodiments, the plasticizer can be triethyl citrate. In certain embodiments, the plasticizer can be dibutyl sebacate.

In certain embodiments, the drug layer can comprise pyridostigmine bromide, ethylcellulose, dibutyl sebacate, and talc. In certain embodiments, the drug layer can comprise pyridostigmine bromide, hypromellose, and talc.

In certain embodiments, the seal coat can comprise at least one water-soluble polymer comprising hypromellose and/or hydroxypropyl cellulose.

In certain embodiments, the functional coat can comprise at least one water-insoluble lipophilic material and, optionally, at least one water-soluble hydrophilic polymer. In certain embodiments, the functional coat can comprise at least one water-insoluble lipophilic polymer and at least one water-soluble hydrophilic polymer (i.e., a pore former).

In certain embodiments, the water-insoluble lipophilic material in the functional coat/membrane can be selected from the group comprising, but not limited to, ethyl acrylate and methyl methacrylate copolymer (EUDRAGIT® NE, EUDRAGIT® NM); a copolymer of ethyl acrylate, methyl methacrylate and trimethylammonioethyl methacrylate chloride (EUDRAGIT® RL 100, EUDRAGIT® RS 100, EUDRAGIT® RL 30D, EUDRAGIT® RS 30D, EUDRAGIT® RL PO, EUDRAGIT® RS PO); carnauba wax; stearic acid; ethylcellulose (ETHOCEL™); cellulose acetate, and polyvinyl acetate dispersion (KOLLICOAT® SR). In certain embodiments, the water-soluble hydrophilic polymer comprises, but is not limited to, polyethylene glycol (PEG 400, PEG 1000, PEG 1450, PEG 3350), hydroxypropyl cellulose, polyvinyl pyrolidone (PVP), KOLLIDON® 30, KOLLICOAT® IR, mannitol, and methylcellulose (METHOCEL™ E3, METHOCEL™ E5, METHOCEL™ E6).

In certain embodiments, the functional coat further can comprise at least one plasticizer and at least one anti-tacking agent. Useful anti-tacking agents can include, but are not limited to, silicon dioxide (SYLOID® 244FP), fumed silica (CAB-O-SIL®), talc, kaolin, and combinations thereof. Useful plasticizers include, but are not limited to, triethyl citrate, triacetin, polyethylene glycol, propylene glycol, and dibutyl sebacate. In certain embodiments, the plasticizer can be triethyl citrate. In certain embodiments, the plasticizer can be dibutyl sebacate.

In certain embodiments, the pellets can be retained in capsules. In certain embodiments, a composition can consist of pellets consolidated into a packed mass for ingestion, even though the packed mass will separate into individual pellets after ingestion. Conventional methods can be used for consolidating the pellets in this manner. For example, the pellets can be placed in gelatin capsules known in the art as “hard-filled” capsules and “soft-elastic” capsules. The compositions of these capsules and procedures for filling them are known among those skilled in drug formulations and manufacture. The encapsulating material should be highly soluble so that the particles are freed and rapidly dispersed in the stomach after the capsule is ingested. In certain embodiments, the pellets can be incorporated directly into food as sprinkles.

In certain embodiments, the present disclosure provides for a pyridostigmine bromide pellet comprising an inert core, a drug layer containing pyridostigmine bromide over the inert core, and a membrane over the drug layer, wherein the membrane comprises a water-insoluble lipophilic polymer and a water-soluble hydrophilic polymer, and wherein the pellet provides extended release, with minimized initial burst release, of pyridostigmine bromide, for at least about 14 hours. In certain embodiments, the water-insoluble lipophilic polymer of the pellet of the present disclosure is selected from the group consisting of an ethyl acrylate and methyl methacrylate copolymer; a copolymer of ethyl acrylate, methyl methacrylate and trimethylammonioethyl methacrylate chloride (EUDRAGIT® RL 100, EUDRAGIT® RS 100, EUDRAGIT® RL 30D, EUDRAGIT® RS 30D, EUDRAGIT® RL PO, EUDRAGIT® RS PO); ethylcellulose, cellulose acetate, polyvinyl acetate, and mixtures thereof. In certain embodiments, the water-soluble hydrophilic polymer of the pellet of the present disclosure is selected from the group consisting of polyethylene glycol, hydroxypropyl cellulose, hydroxymethylcellulose, carboxymethylcellulose, polyvinyl pyrolidone, methylcellulose, xanthan gum, guar gum, sodium alginate, starch, a copolymer of polyvinyl acetate and polyvinyl pyrolidone, a copolymer of ethylene glycol and propylene glycol, a copolymer of polyvinyl alcohol and polyethylene glycol, and mixtures thereof. In certain embodiments, the pellet of the present disclosure further comprises a seal coat between the drug layer and the membrane. In certain embodiments, the seal coat of the pellet of the present disclosure comprises a water-soluble polymer selected from the group consisting of a polyvinyl alcohol-based polymer, methyl cellulose, hydroxyethyl cellulose, hydroxypropyl cellulose, hydroxypropyl methylcellulose, and mixtures thereof. In certain embodiments, the water-soluble polymer of the pellet of the present disclosure is hypromellose, hydroxypropyl cellulose, or a mixture thereof.

In certain embodiments, the pellets can comprise from about 100 mg to about 250 mg, from about 150 mg to about 200 mg, or about 180 mg of pyridostigmine bromide in a pyridostigmine bromide containing core. In certain embodiments, the pellets can comprise a seal coat. In certain embodiments, the seal coat can comprise from about 5 mg to about 30 mg, from about 10 mg to about 20 mg, or about 15 mg of hydroxypropyl cellulose. In certain embodiments, the seal coat can further comprise from about 1 mg to about 10 mg, from about 2 mg to about 5 mg, or about 3 mg of talc. In certain embodiments, the pellets can further comprise a functional coat. The functional coat can comprise from about 10 mg to about 50 mg, from about 20 mg to about 40 mg, or from about 25 mg to about 35 mg of ethyl cellulose. In certain embodiments, the functional coat can further comprise from about 1 mg to about 10 mg, from about 2 mg to about 5 mg, or about 3 mg of triethyl citrate. In certain embodiments, the functional coat can further comprise from about 1 mg to about 10 mg, from about 2 mg to about 5 mg, or about 3 mg of talc. In certain embodiments, the functional coat can further comprise from about 1 mg to about 10 mg, from about 2 mg to about 5 mg, or about 3 mg of hydroxypropyl methylcellulose with a viscosity of between about 4 cp and 6 cp.

In certain embodiments, the present disclosure provides for a pyridostigmine bromide pellet comprising an inert core, a drug layer containing pyridostigmine bromide over the inert core, and a membrane over the drug layer, wherein the membrane comprises a water-insoluble lipophilic polymer and a water-soluble hydrophilic polymer, and wherein the pellet provides extended release, with minimized initial burst release, of pyridostigmine bromide, for at least about 14 hours. In certain embodiments, the water-insoluble lipophilic polymer of the pellet of the present disclosure is selected from the group consisting of an ethyl acrylate and methyl methacrylate copolymer; a copolymer of ethyl acrylate, methyl methacrylate and trimethylammonioethyl methacrylate chloride (EUDRAGIT® RL 100, EUDRAGIT® RS 100, EUDRAGIT® RL 30D, EUDRAGIT® RS 30D, EUDRAGIT® RL PO, EUDRAGIT® RS PO); ethylcellulose; cellulose acetate; polyvinyl acetate, and mixtures thereof. In certain embodiments, the water-soluble hydrophilic polymer of the pellet of the present disclosure is selected from the group consisting of polyethylene glycol, hydroxypropyl cellulose, hydroxymethylcellulose, carboxymethylcellulose, polyvinyl pyrolidone, methylcellulose, xanthan gum, guar gum, sodium alginate, starch, a copolymer of polyvinyl acetate and polyvinyl pyrolidone, a copolymer of ethylene glycol and propylene glycol, a copolymer of polyvinyl alcohol and polyethylene glycol, and mixtures thereof. In certain embodiments, the pellet of the present disclosure further comprises a seal coat between the drug layer and the membrane. In certain embodiments, the seal coat of the pellet of the present disclosure comprises a water-soluble polymer selected from the group consisting of a polyvinyl alcohol-based polymer, methyl cellulose, hydroxyethyl cellulose, hydroxypropyl cellulose, hydroxypropyl methylcellulose, and mixtures thereof. In certain embodiments, the water-soluble polymer of the pellet of the present disclosure is hypromellose, hydroxypropyl cellulose, or a mixture thereof.

In certain embodiments, the pellets can comprise from about 20 mg to about 150 mg, from about 50 mg to about 100 mg, or from about 70 mg to about 80 mg of pyridostigmine bromide granules in a pyridostigmine bromide containing core. In certain embodiments, the pellet can further comprise a drug layer. In certain embodiments, the drug layer can comprise from about 50 mg to about 200 mg, from about 75 mg to about 150 mg, or from about 95 mg to about 105 mg of pyridostigmine bromide. In certain embodiments, the drug layer can further comprise from about 10 mg to about 40 mg, from about 15 mg to about 30 mg, or from about 20 mg to about 25 mg of hydroxypropyl methylcellulose. In certain embodiments, the drug layer can further comprise from about 1 mg to about 10 mg, from about 2 mg to about 8 mg, or from about 3 mg to about 5 mg of talc. In certain embodiments, the pellets can further comprise a seal coat. In certain embodiments, the seal coat can comprise from about 5 mg to about 30 mg, from about 10 mg to about 25 mg, or from about 15 mg to about 20 mg of hydroxypropyl cellulose. In certain embodiments, the seal coat can further comprise from about 1 mg to about 10 mg, from about 2 mg to about 8 mg, or from about 3 mg to about 5 mg of talc. In certain embodiments, the pellets can further comprise a functional coat. In certain embodiments, the functional coat can comprise from about 10 mg to about 100 mg, from about 25 mg to about 80 mg, or from about 50 mg to about 75 mg of ethyl cellulose. In certain embodiments, the functional coat can further comprise from about 2 mg to about 15 mg, from about 2 mg to about 10 mg, or from about 5 mg to about 8 mg of triethyl citrate. In certain embodiments, the functional coat can further comprise from about 2 mg to about 15 mg, from about 2 mg to about 10 mg, or from about 5 mg to about 8 mg of talc.

In certain embodiments, the pellets can comprise a cellet core. In certain embodiments, the pellet comprises about 100 mg of cellet core. In certain embodiments, the pellets can further comprise a drug layer. In certain embodiments, the drug layer can comprise from about 100 mg to about 300 mg, from about 125 mg to about 250 mg, or from about 150 mg to about 200 mg of pyridostigmine bromide. In certain embodiments, the drug layer can further comprise from about 10 mg to about 60 mg, from about 20 mg to about 50 mg, or from about 30 to about 40 mg of ethyl cellulose. In certain embodiments, the drug layer can further comprise from about 1 mg to about 10 mg, from about 2 mg to about 7 mg, or from about 3 mg to about 5 mg of dibutyl sebacate. In certain embodiments, the drug layer can further comprise from about 1 mg to about 15 mg, from about 5 mg to about 10 mg, or about 6 mg of talc. In certain embodiments, the pellet can further comprise a seal coat. In certain embodiments, the seal coat can comprise from about 10 mg to about 100 mg, from about 10 mg to about 85 mg, from about 50 mg to about 75 mg, or from about 15 mg to about 20 mg of hydroxypropyl methylcellulose. In certain embodiments, the seal coat can further comprise from about 1 mg to about 10 mg, from about 2 mg to about 8 mg, or from about 3 mg to about 5 mg of talc. In certain embodiments, the pellet can further comprise a functional coat. In certain embodiments, the functional coat can comprise from about 20 mg to about 120 mg, from about 30 mg to about 100 mg, from about 45 to about 85 mg, or from about 50 mg to about 75 mg of ethyl cellulose. In certain embodiments, the functional coat can further comprise from about 5 mg to about 30 mg, from about 10 mg to about 25 mg, or from about 12 mg to about 18 mg of dibutyl sebacate. In certain embodiments, the functional coat can further comprise from about 1 mg to about 20 mg, from about 5 mg to about 15 mg, or from about 7 mg to about 13 mg of talc. In certain embodiments, the functional coat can further comprise from about 0.5 mg to about 5 mg, from about 1 mg to about 4 mg, or from about 2 mg to about 3 mg of fumed silica. In certain embodiments, the functional coat can further optionally comprise from about 0.5 mg to about 15 mg, from about 1 mg to about 10 mg, or from about 1.5 mg to about 2.5 mg of hydroxypropyl methylcellulose. In certain embodiments, the functional coat can further optionally comprise from about 20 mg to about 150 mg, from about 50 mg to about 120 mg, or from about 75 mg to about 100 mg of cellulose acetate. In certain embodiments, the functional coat can further optionally comprise from about 5 mg to about 40 mg, from about 10 mg to about 25 mg, or from about 15 mg to about 20 mg of polyethylene glycol.

### 6.3. Methods of Making

In certain embodiments, the present disclosure provides extended release pyridostigmine compositions suitable for maintaining stable plasma concentrations, with reduced initial burst release/dose dumping, of pyridostigmine or a pharmaceutically acceptable salt thereof. In certain embodiments, the compositions of the disclosure can provide extended release of pyridostigmine or a pharmaceutically acceptable salt thereof for at least about 14 hours. The extended release pyridostigmine compositions of the disclosure can include matrix tablets, and pellets suitable for dosing in capsules, sachets, and as sprinkles on food. In certain embodiments, the pyridostigmine compositions can comprise gastroretentive tablet compositions providing extended release of pyridostigmine or a pharmaceutically acceptable salt thereof for at least about 14 hours. In certain embodiments, gastroretentive pyridostigmine compositions of the disclosure are suitable for once-daily administration.

In certain embodiments, the pyridostigmine compositions of the disclosure are direct compression tablets. The tablets are made by mixing pyridostigmine bromide, a water-insoluble lipophilic polymer, a filler, a lubricant, and a glidant into a uniform blend; compressing the blend into a tablet core; and coating the tablet core with a functional coat/membrane. In certain embodiments, there is a seal coat between the tablet core and the functional coat. In certain embodiments, the pyridostigmine compositions of the disclosure include pyridostigmine granules that are made by hot-melt extrusion. In certain embodiments, the hot-melt extruded pyridostigmine granules are mixed with extragranular excipients into a uniform blend, and the uniform blend is compressed into a tablet. In certain embodiments, the matrix tablets are further coated with an IR drug layer comprising pyridostigmine bromide and a binder, using a perforated pan coater.

In certain embodiments, the pyridostigmine compositions of the disclosure are gastroretentive tablets. In certain embodiments, the gastroretentive compositions of the disclosure are made by mixing pyridostigmine or a pharmaceutically acceptable salt thereof, an acid, and a gas generating agent, a wicking agent, a filler, and a glidant into a uniform blend; adding lubricant to the resulting blend and compressing the blend into a tablet core; coating the seal-coated tablets with a functional coat comprising a plasticizer, and at least one of ethyl acrylate and methyl methacrylate copolymer (EUDRAGIT® NE, EUDRAGIT® NM) or a copolymer of ethyl acrylate, methyl methacrylate and trimethylammonioethyl methacrylate chloride (EUDRAGIT® RL 100, EUDRAGIT® RS 100, EUDRAGIT® RL 30D, EUDRAGIT® RS 30D, EUDRAGIT® RL PO, EUDRAGIT® RS PO). In certain embodiments, the functional coat can comprise a plasticizer, and at least one copolymer of ethyl acrylate, methyl methacrylate and trimethylammonioethyl methacrylate chloride (e.g., EUDRAGIT® RL 100, EUDRAGIT® RS 100, EUDRAGIT® RL 30D, EUDRAGIT® RS 30D, EUDRAGIT® RL PO, EUDRAGIT® RS PO). In certain embodiments, the gastroretentive tablets can be further coated with an IR drug layer comprising pyridostigmine bromide and a binder, using a perforated pan coater.

In certain embodiments, the disclosure provides a method for making gastroretentive dosage form comprising a core and a permeable elastic membrane containing an orifice and surrounding the core, the method comprises, mixing pyridostigmine or a pharmaceutically acceptable salt thereof with a glidant(s) to obtain a drug intermediate blend; mixing the drug intermediate blend with an acid(s), a gas generating agent, and a swellable water soluble polymer(s) into a final blend; compressing the final blend into a tablet core; coating the tablet core with a functional coat/permeable elastic membrane comprising at least one copolymer of ethyl acrylate, methyl methacrylate, and trimethylammonioethyl methacrylate chloride, and a plasticizer(s), to obtain a membrane coated tablet core; and drilling an orifice through the membrane/functional coat. In certain embodiments, the final blend further comprises a filler(s), a wicking agent(s), a glidant(s), an anti-tacking agent(s), and a lubricant(s). In certain embodiments, the functional coat is further coated with a cosmetic coat/over coat. In certain embodiments, there is a seal coat between the core and the functional coat. In certain embodiments, the is a seal coat between the core and the functional coat. In certain embodiments, seal coat comprises water soluble polymer selected from the group consisting of a polyvinyl alcohol-based polymer (OPADRY® white, OPADRY® clear), methyl cellulose, hydroxyethyl cellulose, hydroxypropyl cellulose, hydroxypropyl methylcellulose, and mixtures thereof. In certain embodiments, the over coat comprises water-soluble hydrophilic polymers selected from the group consisting of methylcellulose, hydroxymethyl cellulose, hydroxypropyl cellulose, hydroxypropyl methylcellulose, polyvinyl alcohol-based OPADRY® white, and mixture thereof.

In certain embodiments, the disclosure provides a method for making a gastroretentive dosage form comprising an immediate release portion comprising an immediate release drug layer; and an extended release portion comprising a core coated with a permeable elastic membrane comprising an orifice. The method comprises mixing pyridostigmine or a pharmaceutically acceptable salt thereof with a glidant(s) to obtain a drug intermediate blend, mixing the drug intermediate blend with an acid(s), a gas generating agent, and a swellable water soluble polymer(s) into a final blend, compressing the final blend into a tablet core, coating the tablet core with a permeable elastic membrane comprising at least one copolymer of ethyl acrylate, methyl methacrylate, and trimethylammonioethyl methacrylate chloride, and a plasticizer(s), to obtain a membrane coated tablet core, drilling an orifice through the membrane to a obtain membrane coated tablet core comprising an orifice in the membrane, and coating the membrane coated tablet core comprising an orifice in the membrane with an immediate release drug layer comprising pyridostigmine or a pharmaceutically acceptable salt thereof. In certain embodiments, the immediate release drug layer is further coated with a cosmetic coat/over coat. In certain embodiments, there is seal coat-1 between functional coat/membrane and immediate release drug layer and seal coat-2 between immediate release drug layer and over coat/cosmetic coat. In certain embodiments, seal coat comprises water soluble polymer selected from the group consisting of a polyvinyl alcohol-based polymer (OPADRY® white, OPADRY® clear), methyl cellulose, hydroxyethyl cellulose, hydroxypropyl cellulose, hydroxypropyl methylcellulose, and mixtures thereof. In certain embodiments, the over coat comprises water-soluble hydrophilic polymers selected from the group consisting of methylcellulose, hydroxymethyl cellulose, hydroxypropyl cellulose, hydroxypropyl methylcellulose, polyvinyl alcohol-based OPADRY® white, and mixture thereof.

In certain embodiments, the orifice passes through seal coat-1 and functional coat.

In certain embodiments, the pyridostigmine compositions of the disclosure can comprise pyridostigmine pellets suitable for dosing in capsules, sachets, and as sprinkles on food. In certain embodiments, the pellets can comprise a pyridostigmine bromide core. In certain embodiments, the pellets can comprise a cellet. In certain embodiments, the pellet cores (e.g., pyridostigmine bromide cores or cellets) are further drug-layered with pyridostigmine bromide. In certain embodiments, the pellets are made by coating the pyridostigmine bromide core with a seal coat comprising a water-soluble hydrophilic polymer; coating the seal-coated pellets with a functional coat comprising a plasticizer, a water-insoluble lipophilic polymer that is insoluble in physiological fluids, and a pore former comprising a water-soluble hydrophilic polymer. In certain embodiments, the pyridostigmine bromide cores are further drug-layered with pyridostigmine bromide.

In certain embodiments, various solvents used in processes of the disclosure include, but are not limited to, water, methanol, ethanol, acetone, isopropyl alcohol, and mixtures thereof. In certain embodiments, the solvent is a mixture of acetone and water, a mixture of ethanol and isopropyl alcohol, a mixture of acetone and isopropyl alcohol, a mixture of isopropyl alcohol and water, or a mixture of water, ethanol, and isopropyl alcohol. In certain embodiments, the solvent is a mixture of acetone and water. In certain embodiments, the ratio of solvent and water ranges from about 70:30 to about 99:1. In certain embodiments, the ratio of acetone and water is about 70:30, about 75:25, about 80:20, about 85:15, about 90:10, about 95:5, or intermediate ranges therein.

### 6.4 Orthostatic Hypotension

Orthostatic hypotension (OH) refers to fall in blood pressure on standing that can result in hypoperfusion of organs, including brain. In certain embodiments, orthostatic hypotension (OH) refers to a sustained reduction of systolic blood pressure (SBP) by at least 20 mmHg and/or or diastolic blood pressure (DBP) by at least 10 mmHg, within 3 min of standing or head-up tilt to at least 60° on a tilt table.

Neurogenic orthostatic hypotension (nOH) refers to OH caused by impairment of autonomic nervous system characterized by failure to provide adequate autonomic postural responses, most prominently systemic vasoconstriction and compensatory increase in heart rate (HR) sufficient to maintain blood pressure.

Orthostatic hypotension may be neurogenic hypotension (nOH). Neurogenic orthostatic hypotension can result from failure to release required amount of norepinephrine upon standing, primary autonomic failure (central and peripheral synucleinopathies), parkinson's disease, multiple system atrophy (MTA), pure autonomic failure (PAF), autonomic neuropathy, or a combination thereof. Additionally, dopamine ß-hydroxylase deficiency, and diabetic and nondiabetic autonomic neuropathy can also cause nOH. In patients with multiple system atrophy and Parkinson's disease, nOH can occur early and may precede other manifestations of the disease.

Symptoms of nOH may include retinal hypoperfusion, muscle hypoperfusion, lung hypoperfusion, cerebral hypoperfusion, myocardial hypoperfusion, nonspecific symptoms, or a combination thereof. Such symptoms may vary from patient to patient. For example, patients with retinal hypoperfusion may exhibit symptoms of impaired vision; patients with muscle hypoperfusion may exhibit symptoms of neck and shoulder pain (“coat hanger” ache); patients with lung hypoperfusion may exhibit symptoms of orthostatic dyspnea; patients with cerebral hypoperfusion may exhibit symptoms of dizziness, light headedness, presyncope, syncope, difficulty concentrating, headache, and cognition; patients with myocardial hypoperfusion may exhibit symptoms of angina; and sometimes patients may exhibit nonspecific symptoms, for example generalized weakness, falls, leg buckling, lethargy, fatigue, and nausea, and increased risk for fractures and head trauma.

Dose titration and symptoms of OH may be monitored by measured of standing blood pressure following administration of pyridostigmine compositions of the disclosure.

### 6.5 Methods of Treatment, Dosage, and Administration

In certain embodiments, pyridostigmine compositions of the disclosure are supplied in 105 mg, 205, mg, 275 mg, and 340 mg tablet strengths. In certain embodiments, the 105 mg tablet is a peach, oval, coated, un-scored, biconvex tablet, printed with dose strength on one side and plain on the other side. A hole may be visible on one side of the tablet. In certain embodiments, the 205 mg tablet is a powder blue, oval, coated, un-scored, biconvex tablet, printed with dose strength on one side and plain on the other side. A hole may be visible on one side of the tablet. In certain embodiments, the 270 mg tablet is a light pink, oval, coated, un-scored, biconvex tablet, printed with dose strength on one side and plain on the other side. A hole may be visible on one side of the tablet. In certain embodiments, the 340 mg tablet is a yellow, oval, coated, un-scored, biconvex tablet, printed with dose strength on one side and plain on the other side. A hole may be visible on one side of the tablet. In certain embodiments, thirty tablets are packed along with one or more SG CSF canister, in a high density polyethylene (HDPE) screw-cap induction sealed close bottle with a child resistance polypropylene screw cap. In certain embodiments, thirty tablets are packed along with 4 counts of 3 gm SG CSF canister, in a white, wide mouth, pharmaceutical round, 150 cc high density polyethylene (HDPE) screw-cap induction sealed close bottle with a child resistance polypropylene screw cap.

In certain embodiments, the tablets are packed in a blister or strip package. In certain embodiments, the blister or strip package is supplied in a protective sleeve. In certain embodiments, tablets are blister packed using Alu-Alu cold forming material and peel-push/push through lidding material. In certain embodiments, tablets are blister packed using Aclar® TX H300 thermo-forming material and peel-push/push through lidding material. In certain embodiments, protective sleeve is a cardboard sleeve. In certain embodiments, one or more protective sleeves are placed in a Mylar bag with a desiccant. In certain embodiments, the 105 mg pyridostigmine compositions of the disclosure are packed in a blister or strip package supplied in a protective sleeve. In certain embodiments, protective sleeve is a cardboard sleeve. In certain embodiments, one or more protective sleeves are placed in a Mylar bag with a desiccant. In certain embodiments, tablets (e.g., 7 count or 10 count) are blister packed using cold-forming or thermo-forming materials. In certain embodiments, tablets are blister packed using Alu-Alu cold forming material and peel-push/push through lidding material. In certain embodiments, tablets are blister packed using Aclar® TX H300 thermo-forming material and peel-push/push through lidding material. In certain embodiments, 7 tablets are blister packed using Aclar® TX H3000, thermo-forming material and push through lidding material. In certain embodiments, each blister pack is placed in a cardboard sleeve, and 10 cardboard sleeves are placed in a Mylar bag with a desiccant.

In certain embodiments, the disclosure provides for methods of treating a patient or a person in need thereof, Myasthenia Gravis (MG), Lambert-Eaton Myasthenic Syndrome (LEMS), postoperative bowel bloating, urinary retention, orthostatic hypotension (OH), and/or neurogenic orthostatic hypotension (nOH) in patients suffering from autonomic diseases caused by primary autonomic failure, e.g., Parkinson's disease, multiple system atrophy, and pure autonomic failure.

In certain embodiments, the disclosure provides methods for treating Myasthenia Gravis. In certain embodiments, the method comprises symptomatic treatment of Myasthenia Gravis in a patient. In certain embodiments, the symptomatic treatment comprises alleviation, mitigation, and/or decrease in at least one clinical symptom in the patient. In certain embodiments, the clinical symptom can comprise muscle weakness, weakness of the eye muscles (e.g., Ocular Myasthenia), drooping of one or both eyelids (Ptosis), blurred or double vision (e.g., Diplopia), change in facial expression, difficulty swallowing, shortness of breath, impaired speech (dysarthria), and/or weakness in arms, hands, fingers, legs, and/or neck.

In certain embodiments, the disclosure provides methods for treating nOH. In certain embodiments, the method comprises treatment of symptoms of nOH caused by primary autonomic failure [Parkinson's disease (PD), multiple system atrophy and pure autonomic failure, dopamine beta-hydroxylase deficiency, drug induced orthostatic hypotension, and diabetic non-diabetic autonomic neuropathy. In certain embodiments, symptoms of nOH comprise orthostatic dizziness, lightheadedness, feeling like you might black out, cognitive slowing, sleepiness, presyncope, and syncope, increased risk of falls, cognitive impairment, and exercise intolerance. In certain embodiments, the disclosure provides methods for treating symptoms of nOH without worsening supine hypertension (SH). In certain embodiments, the disclosure provides methods for treating symptoms of nOH while reducing the occurrence of SH. In certain embodiments, the disclosure provides for a method for treating neurogenic hypotension without worsening supine hypertension. In certain embodiments, the patients with nOH are advised to elevate the head of the bed when resting or sleeping, to minimize the risk of SH.

In certain embodiments, the disclosure provides a method of pretreatment for exposure to the chemical nerve agent Soman. In certain embodiments, the disclosure provides a method for treating or preventing organophosphorus or nerve gas poisoning or injuries.

In certain embodiments, the disclosure provides a method for treating dementia, including Alzheimer's disease.

In certain embodiments, the methods comprise administering to a patient or a person in need thereof, an extended release pyridostigmine composition of the disclosure. The dosage frequency of administration depends upon the requirement of clinical response of the patient or a person in need thereof. The dosage schedule is adjusted for each patient and changed as the need arises. In certain embodiments, dosage requirements in patients with Myasthenia Gravis may vary from day to day, according to remissions and exacerbations of the disease and physical and emotional stress suffered by the patient. In certain embodiments, the daily dose is administered when the patient is more prone to fatigue. In certain embodiments, the dosage form is suitable for once daily administration. In certain embodiments, the dosage form is suitable for twice daily administration (e.g., where higher doses, e.g., above 270 mg or above 340 mgs are required). In certain embodiments, the dosage forms of the present disclosure are administered QD as a single dosage unit. In certain embodiments, the compositions of the disclosure are administered QD as multiple dosage units (e.g., two, three, or four dosage units). In certain embodiments, the dose strength and dosing frequency is determined based on the condition being treated and the severity of the condition. In certain embodiments, during initial treatment of Myasthenia Gravis/clinical symptoms of Myasthenia Gravis, the treatment is started at a dosage smaller than that required to produce maximum strength, and daily dosage gradually increased to intervals of 48 hours or more. Changes in oral dosage may take several days to show results. In certain embodiments, when a further increase in dosage produces no corresponding increase in muscle strength, dosage should be reduced the previous level so that the patient receives the smallest dose necessary to produce maximum strength.

In certain embodiments, the disclosure provides a method for improving patient compliance by administering extended release pyridostigmine compositions of the disclosure, wherein the compositions provide an extended release, with reduced initial burst release compared to marketed extended release pyridostigmine products. In certain embodiments, the extended release pyridostigmine compositions of the disclosure improve patient compliance by including an IR drug layer that provides a drug plasma concentration sufficient to overcome the lag time in pyridostigmine release seen without application of an IR layer, and sufficient to provide instant therapeutic effects, with reduced or eliminated GI side effects; the extended release component provides extended release of the drug for a period of at least about 14 hours.

In certain embodiments, the disclosure provides a method for improving patient compliance by administering extended release pyridostigmine compositions of the disclosure, wherein the extended release compositions will allow for reduced frequency of administration of the composition. In certain embodiments, the extended release pyridostigmine compositions of the disclosure reduce pill-burden compared to the approved pyridostigmine compositions, e.g., MESTINON® and MESTINON® TIMESPAN®. According to the prescribing information for MESTINON®, the average daily dose of pyridostigmine is ten 60 mg tablets, ten teaspoons of suspension, or between one and three 180 mg ER tablets, spaced to provide maximum relief. The ER 180 mg tablets are administered, as 1-3 tablets, depending upon severity of the condition, once- or twice-daily with an interval of at least 6 hours between doses. Additionally, immediate release pyridostigmine tablets or oral solution may be required in conjunction with MESTINON® TIMESPAN® therapy. Such dosing regimens are challenging for patient adherence, which results in “wearing off” effect, symptom worsening, and acute cholinergic side effects. The extended release compositions of pyridostigmine compositions of the disclosure are suitable for once-daily dosing, thereby significantly reducing the pill-burden compared to marketed pyridostigmine products, e.g., MESTINON and MESTINON TIMESPAN®.

In certain embodiments, the dosage forms of the disclosure reduce initial burst initial burst release while providing a therapeutically effective plasma concentration of pyridostigmine or a pharmaceutically acceptable salt thereof for periods of about 12 hours to about 24 hours.

In certain embodiments, the disclosure provides methods for improving patient compliance, with once-a-day administration of extended release pyridostigmine compositions of the disclosure, by reducing initial burst release of pyridostigmine or a pharmaceutically acceptable salt thereof, and providing the desired therapeutic effect with minimal side effects including nausea, vomiting, diarrhea, abdominal cramps, fasciculations, increased peristalsis, increased salivation, increased bronchial secretions, miosis, and diaphoresis.

In certain embodiments, the extended release pyridostigmine compositions of the disclosure provide 24-hour symptom control, tolerability, and reduced pill-burden by maintaining therapeutic plasma concentration of pyridostigmine over the 24-hour dosing period. In certain embodiments, the extended release pyridostigmine compositions of the disclosure provide 24-hour symptom control, e.g., providing nighttime and early morning functionality, by maintaining therapeutic plasma levels during the 24-hour dosing period. In certain embodiments, the extended release pyridostigmine compositions of the disclosure provide residual plasma levels of the drug in the morning, such that patients wake up feeling more refreshed and more functional before taking the morning dose, as compared with the currently marketed pyridostigmine products.

In certain embodiments, the extended release pyridostigmine compositions of the disclosure provide lower Fluctuation Index over a single dosing period, compared to marketed pyridostigmine products, e.g., MESTINON® and MESTINON® TIMESPAN®, to provide a 24-hour release profile with superior all day coverage and improved tolerability compared to the marketed pyridostigmine products. In certain embodiments, the gastroretentive dosage forms of the present disclosure provide extended release of pyridostigmine or a pharmaceutically acceptable salt thereof for at least about 14 hours, e.g., for up to about 24 hours.

In certain embodiments, the extended release pyridostigmine compositions of the disclosure reduce worsening of symptoms and improve quality of life by increasing the Cmin/trough levels. In certain embodiments, higher Cmin/trough level of extended release pyridostigmine compositions of the disclosure, compared to the marketed pyridostigmine products, e.g., MESTINON® and MESTINON® TIMESPAN®, reduce wearing off effects and worsening of symptoms, e.g., provide a better 24-hour control of Myasthenia Gravis symptoms.

In certain embodiments, the extended release pyridostigmine compositions of the disclosure improve tolerability and reduce side effects by reducing the initial burst release and Fluctuation Index. In certain embodiments, reduced initial burst release in extended release pyridostigmine compositions of the disclosure, as compared to the marketed pyridostigmine products, e.g., MESTINON® and MESTINON® TIMESPAN®, reduce cholinergic side effects, e.g., muscle cramps, and provide better 24-hour control of Myasthenia Gravis symptoms.

In certain embodiments, the present disclosure provides for a therapeutic method for symptom treatment or treatment for Myasthenia Gravis, the method comprising orally administering to a patient or a person in need thereof a single QD gastroretentive pyridostigmine tablet, wherein the tablet provides an extended release, with release of less than 35 wt % of pyridostigmine or a pharmaceutically acceptable salt thereof, within two hours of administration. In certain embodiments, the present disclosure provides for a method for reducing GI side effects in a patient consuming a pyridostigmine composition, the method comprising administering to the patient a pyridostigmine composition of the disclosure comprising an immediate release drug layer and an extended release component, wherein the composition provides a reduced initial burst release comprising release of less than 35 wt % of pyridostigmine or a pharmaceutically acceptable salt thereof, within two hours of administration.

In certain embodiments, the present disclosure provides for a method for improving patient compliance in a patient consuming a pyridostigmine composition, the method comprising administering to the patient a gastroretentive pyridostigmine composition comprising an immediate release layer and an extended release component as a single tablet/day, wherein the composition provides extended release with reduced initial burst release of pyridostigmine or a pharmaceutically acceptable salt thereof, wherein the reduced initial burst release comprises a release of less than 35 wt % of pyridostigmine or a pharmaceutically acceptable salt thereof within 2 hours of administration. In certain embodiments, the reduced initial burst release of less than 35 wt % of pyridostigmine or a pharmaceutically acceptable salt thereof within two hours of administration is equivalent to an in vitro release of less than 35 wt % of pyridostigmine or a pharmaceutically acceptable salt thereof, as measured in 900 ml of a dissolution medium with pH of less than about 5.5 and containing from about 1 mM to about 200 mM of NaCl, measured using USP Apparatus I, at about 100 rpm and about 37° C.

### 7. EXAMPLES

The following examples illustrate the disclosure in a nonlimiting manner. Unless indicated to the contrary, the numerical parameters set forth herein can vary depending upon the desired properties sought to be obtained by the present disclosure.

### Example 1: Pyridostigmine Bromide Matrix Tablet Compositions (180 mg)

The present example provides various formulations for pyridostigmine bromide tablets as outlined in Table 1 and Table 2. Seven different tablets were prepared.

TABLE 1
Formulation of Matrix Tablets

| | Tablet | Tablet | Tablet | Tablet | Tablet |
|---|---|---|---|---|---|
| | 1 | 2 | 3 | 4 | 5 |
| Ingredients | (mg) | (mg) | (mg) | (mg) | (mg) |
| Pyridostigmine | 180.0 | 180.0 | 180.0 | 180.0 | 180.0 |
| bromide | | | | | |
| Stearic acid | — | 180.0 | — | 20.00 | 20.00 |
| Carnauba wax | — | — | — | 160.00 | 80.00 |
| Ethylcellulose | — | — | — | — | — |
| (ETHOCEL ™) | | | | | |
| Silicon dioxide | 180.0 | — | 180.0 | — | 80.00 |
| (SYLOID ® 244FP) | | | | | |
| Fumed silica | — | 20.00 | 20.00 | 20.00 | 20.00 |
| (CAB-O-SIL ®) | | | | | |
| Mannitol (PARTECK ® M200) | 100.0 | 100.0 | 100.0 | 100.0 | 100.0 |
| Magnesium stearate | 5.00 | 5.00 | 5.00 | 5.00 | 5.00 |
| Core Tablet Weight | 465.0 | 485.0 | 485.0 | 485.0 | 485.0 |

Functional Coat

| | | | | | |
|---|---|---|---|---|---|
| Cellulose acetate | 40.40 | 41.73 | 41.73 | 41.73 | 41.73 |
| Polyethylene | 2.02 | 2.08 | 2.08 | 2.08 | 2.08 |
| glycol (PEG 3350) | | | | | |
| Methylcellulose | 4.004 | 4.173 | 4.173 | 4.173 | 4.173 |
| (METHOCEL ™ E5) | | | | | |

Solvent* acetone:water (95:5)

| | | | | | |
|---|---|---|---|---|---|
| Total Tablet Weight | 511.42 | 532.98 | 532.98 | 532.98 | 532.98 |

*Removed during process

TABLE 2
Formulation of Matrix Tablets

| | Tablet 6** | Tablet 7 |
|---|---|---|
| Ingredients | (mg) | (mg) |
| Pyridostigmine bromide | 150.00 | 180.00 |
| Stearic acid | — | 90.00 |
| Ethylcellulose (ETHOCEL ™) | 100.00 | — |
| Silicon dioxide (SYLOID ® 244FP) | 20.00 | — |
| Fumed silica (CAB-O-SIL ®) | — | 10.00 |
| Mannitol (PARTECK ® M200) | 100.00 | 100.00 |
| Magnesium stearate | 5.00 | 5.00 |
| Core Tablet Weight | 375.00 | 385.00 |

Functional Coat

| | | |
|---|---|---|
| Cellulose acetate | 62.50 | 33.40 |
| Polyethylene glycol (PEG 3350) | 6.25 | 1.67 |
| Methylcellulose (METHOCEL ™ E5) | 6.25 | 3.34 |

Solvent* acetone:water (95:5)

| | | |
|---|---|---|
| Total Weight | 450.00 | 423.41 |

*Removed during process
**Tablet 6 can have an IR coat of 30 mg of pyridostigmine bromide

Tablets 1-5 and Tablet 7 contain 180 mg of pyridostigmine bromide and include 10% coating weight gain of uncoated tablet. Tablets 1-5 and 6 do not contain ethylcellulose. Tablet 6 contains 150 mg of pyridostigmine bromide, ethylcellulose, and 20% coating weight gain of uncoated tablet. Tablets 1-7 were made according to the following general procedure.

### Manufacturing Procedure:

1. A uniform blend of pyridostigmine, stearic acid, carnauba wax, ethylcellulose, silicon dioxide, fumed silica, mannitol, and magnesium stearate was made as per Tablets 1-7.
2. For each blend, the drug and the excipients were taken in a V-blender and mixed to obtain a uniform blend.
3. Magnesium stearate was sieved through sieve #30 and mixed with the uniform blend from step #2.
4. Required amount of blend was filled into the die and then compressed as tablet compositions.
5. Cellulose acetate and methylcellulose were added to a stainless steel container charged with an acetone and water mixture in a ratio of 95:5 and mixed to obtain a clear solution.
6. Polyethylene glycol 3350 was added to the solution from step #4 and mixed for not less than about 30 minutes.
7. The tablets from step #4 were taken in a coating pan and coated with the solution from step #6 until the target weight gain was attained.

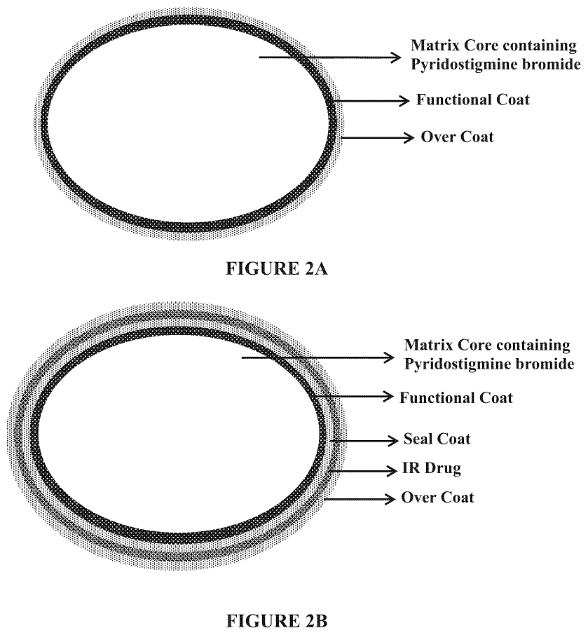

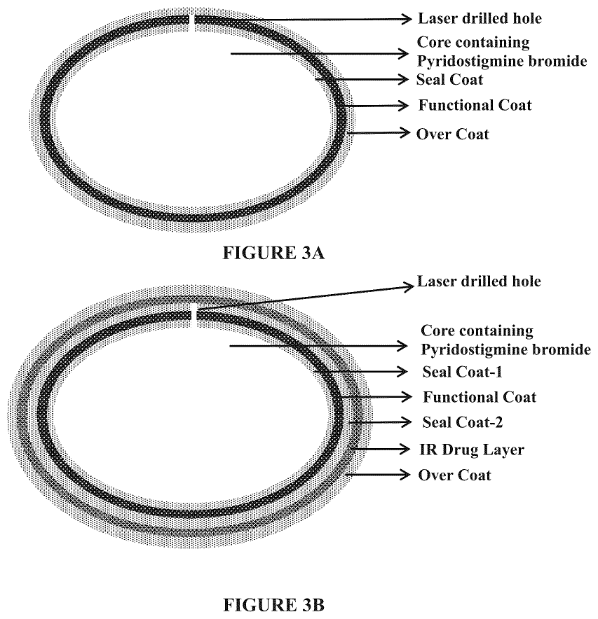

FIG. 2 depicts a schematic representation of pyridostigmine matrix tablets, with and without an immediate release drug layer.

### Example 2: Pyridostigmine Bromide Gastroretentive Tablet Compositions

The present Example provides various formulations for pyridostigmine bromide gastroretentive tablets as outlined in Table 3.

TABLE 3
Formulation of Pyridostigmine Bromide Tablets

| | Tablet | Tablet | Tablet | Tablet | Tablet |
|---|---|---|---|---|---|
| | 8 | 9 | 10 | 11 | 12 |
| | (mg/ | (mg/ | (mg/ | (mg/ | (mg/ |
| Ingredients | dose) | dose) | dose) | dose) | dose |
| Pyridostigmine | 180.0 | 180.0 | 180.0 | 135.0 | 135.0 |
| bromide | | | | | |
| Succinic acid, | 50.0 | 50.0 | 50.0 | 80.0 | 80.0 |
| NF-micronized | | | | | |
| Sodium bicarbonate | 50.00 | 50.0 | 50.0 | 55.0 | 55.0 |
| Calcium carbonate | 125.0 | 125.0 | 125.0 | 65.0 | 65.0 |
| Crospovidone | 100.0 | 100.0 | 100.0 | 200.0 | 100.0 |
| Mannitol (PARTECK ® M200) | 233.0 | 233.0 | 233.0 | 153.0 | 253.0 |
| BENECEL ™ | 200.0 | 300.0 | 200.0 | — | 100.0 |
| K4M PH DC | | | | | |
| BENECEL ™ | — | — | 25.0 | — | — |
| K200M | | | | | |
| METHOCEL ™ | — | — | — | 300.0 | 200.0 |
| K100 Prem LVCR | | | | | |
| CAB-O-SIL ® | 4.00 | 4.00 | 4.00 | 4.00 | 4.00 |
| Magnesium stearate | 8.00 | 8.00 | 8.00 | 8.00 | 8.00 |
| Total weight | 950.0 | 1050.0 | 975.0 | 1000.0 | 1000.0 |

Seal Coat

| | | | | | |
|---|---|---|---|---|---|
| Hydroxypropyl | 33.33 | 33.33 | 33.33 | — | — |
| cellulose | | | | | |
| Talc | 3.33 | 3.33 | 3.33 | — | — |
| Triethyl citrate | 3.33 | 3.33 | 3.33 | — | — |
| Solvent* | — | —. | — | — | —. |
| acetone:water | | | | | |
| (95:5) | | | | | |

Functional Coat

| | | | | | |
|---|---|---|---|---|---|
| EUDRAGIT ® | 148.15 | 148.15 | 148.15 | 148.15 | 148.15 |
| RL PO | | | | | |
| Triethyl citrate | 22.22 | 22.22 | 22.22 | 22.22 | 22.22 |
| Talc | 29.63 | 29.63 | 29.63 | 29.63 | 29.63 |
| Solvent* | — | — | — | — | — |
| acetone:water | | | | | |
| (95:5) | | | | | |

Over Coat

| | | | | | |
|---|---|---|---|---|---|
| OPADRY ® | 15.00 | 15.00 | 15.00 | — | — |
| white | | | | | |
| Solvent*Purified | — | — | — | — | — |
| water, USP | | | | | |
| Total weight | 1205.00 | 1305.00 | 1230.00 | 1200.0 | 1200.0 |

*Removed during process

Tablets 8-10 contain 180 mg of pyridostigmine, 50 mg of succinic acid, 50 mg of sodium bicarbonate, 125 mg of calcium carbonate, and BENECEL™ K4M-DC. Tablet 10 further contains BENECEL™ 200M. Tablets 11-12 contain 135 mg of pyridostigmine bromide, and 80 mg of succinic acid, 55 mg of sodium bicarbonate, and 65 mg of calcium carbonate. Further, Tablet 11 contains METHOCEL™ K100 Premium LVCR and Tablet 12 contains a mixture of METHOCEL™ K100 Premium LVCR and BENECEL™ K4M PH DC. Tablets 8-12 were made according to the following general procedure.

### Manufacturing Procedure:

### A. Core Tablets

1. Pyridostigmine, succinic acid, sodium bicarbonate, calcium carbonate, crospovidone, Mannitol, BENECEL™ K4M-DC, BENECEL™ K200M, METHOCEL™ K100 Premium LVCR, and CAB-O-SIL®, as per Tablets 8-12, were sieved through sieve #20 and mixed to obtain a uniform blend.
2. Magnesium stearate was sieved through sieve #30 and mixed with the uniform blend from step #1.
3. The resulting blend from step #2 was compressed to obtain pyridostigmine tablet cores.

### B. Seal Coating

1. Hydroxypropyl cellulose, triethyl citrate, and talc were added to a mixture of acetone and water (95:5) in a stainless steel container and mixed to form a uniform dispersion.
2. Tablet cores 8-10 were seal coated using a perforated pan coater with an inlet air temperature of 25° C.-60° ° C. at a product temperature of 25-45° C.
3. The coated tablet cores were dried in the coating pan.

### C. Functional Coating and Over Coat

1. EUDRAGIT® RL PO was added to acetone and water mixture (95:5) and mixed to obtain a clear solution.
2. To the solution from step #1, triethyl citrate was added and mixed for at least 45 minutes.
3. To the solution from step #2, talc was added and mixed for at least 60 minutes to obtain a homogeneous dispersion.
4. The homogeneous dispersion from step #3 was sprayed onto the seal coated tablet cores 8-10 and tablet cores without seal coat, e.g., Tablet cores 11-12.
5. The coated tablets from step #4 were dried in a coating pan.
6. An orifice was laser drilled in the coated tablets from step #5 such that the orifice passed through various coating layers.
7. Weighed quantity of opadry white was added into the required amount of purified water. The suspension was mixed until a uniform dispersion was formed.
8. The functional coated tablets from step #6 were further coated with the dispersion from step #7 in a perforated coating pan with inlet air temperature at 25°-45° C.
9. The coated tablets from step #8 were dried in a pan to a moisture content below 3.0%, as measured by loss on drying at 105° C.

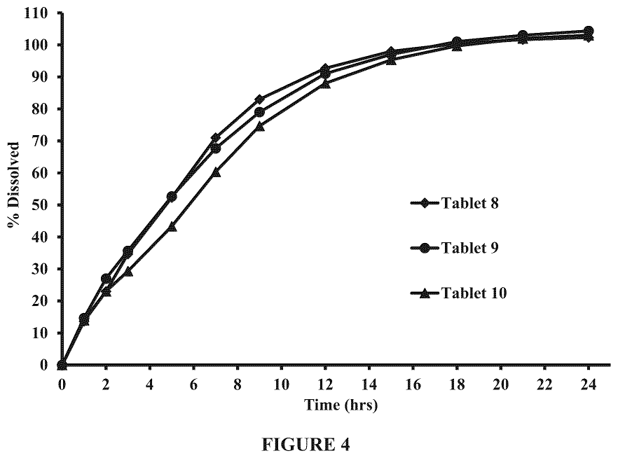

FIG. 4 provides a comparison of dissolution profiles of pyridostigmine bromide gastroretentive Tablets 8, 9 and 10, using USP I-custom basket-dissolution apparatus, in 900 ml 50 mM pH 4.5 acetate buffer, at 100 RPM and 37° C. FIG. 4 demonstrates that Tablets 8-10 provide extended release, with minimized initial burst release, of pyridostigmine bromide for a period of about 22 hours.

### Example 3: Pyridostigmine Bromide Pellet Composition Comprising Pyridostigmine Bromide Granule Core

The present Example provides for a pyridostigmine bromide pellet composition comprising a pyridostigmine bromide core as outlined in Table 4.

TABLE 4
Formulation of Pyridostigmine Bromide Pellet

| | Pellet 1 |
|---|---|
| Ingredients | mg/dose |
| Pyridostigmine bromide granules | 180.00 |

Seal Coat

| | |
|---|---|
| Hydroxypropyl cellulose (KLUCEL ™) | 15.00 |
| Talc | 3.00 |

Functional coat

| | |
|---|---|
| Ethyl cellulose (ETHOCEL ™ 20 cp) | 30.40 |
| Triethyl citrate | 3.00 |
| Talc | 3.00 |
| METHOCEL ™ E5 Premium LV | 3.00 |

Solvent* ethanol:water (90:10)

| | |
|---|---|
| Total Weight | 237.40 |

*Removed during process

Pellet 1 contains pyridostigmine bromide granule as pellet core, and a functional coat comprising ETHOCEL™ 20 cp, METHOCEL™ E5 Premium LV, and triethyl citrate. Pellet 1 was made according to the following general procedure.

### Manufacturing Procedure:

### A. Seal Coating

1. Hydroxypropyl cellulose was added to dehydrated alcohol in a stainless steel container and mixed to form a uniform solution.
2. To the dispersion from step #1, the purified water was added and mixed until a clear solution formed.
3. To the solution from step #2, talc was added and mixed for not less than about 30 minutes to form a homogenous dispersion.
4. Pyridostigmine bromide granules were coated using a Wurster fluid bed coater with an inlet air temperature of 30-50° C., and sufficient air volume for fluidization. When the product temperature reached 30° C., the dispersion from step #3 was sprayed onto the granules while maintaining the product temperature of 25-35° C. and sufficient air volume for the fluidization, until the target coating weight gain was achieved.

### B. Functional Coating

1. Ethyl cellulose and METHOCEL™ E5 Premium LV were added to dehydrated alcohol in a stainless steel container and mixed for about 1 hour to form a uniform dispersion.
2. To the dispersion from step #1, water was added and mixed to obtain a homogeneous dispersion.
3. To the dispersion from step #2, TEC was added and mixed for not less than about 15 minutes.
4. To the dispersion from step #3, talc was added and mixed for not less than about 30 minutes to obtain a uniform dispersion.
5. Seal coated pyridostigmine bromide granules (procedure A, above) were taken in a Wurster chamber and coated with the dispersion from step #4, until target coating weight gain was achieved.

### Example 4: Pyridostigmine Bromide Pellet Composition Comprising Pyridostigmine Bromide Granule Core and Drug Layer Containing Pyridostigmine Bromide

The present Example provides for a pyridostigmine bromide pellet composition comprising a pyridostigmine bromide granule core and a drug layer containing pyridostigmine bromide. Two different pellets were prepared as outlined in Table 5.

TABLE 5
Formulation of Pyridostigmine Bromide Pellet

| | Pellet 2 | Pellet 3 |
|---|---|---|
| Ingredients | mg/dose | mg/dose |
| Pyridostigmine bromide granules | 78.16 | 78.16 |

Drug Layer

| | | |
|---|---|---|
| Pyridostigmine bromide | 101.84 | 101.84 |
| METHOCEL ™ E5 Premium LV | 20.36 | 20.36 |
| Talc | 4.07 | 4.07 |

Solvent* ethanol:water (85:15)
Seal Coat

| | | |
|---|---|---|
| Hydroxypropyl cellulose (KLUCEL ™ LF) | 17.04 | 17.04 |
| Talc | 3.40 | 3.40 |

Solvent* acetone:water (95:5)
Functional Coat

| | | |
|---|---|---|
| Ethyl cellulose (ETHOCEL ™ 20 cp) | 56.20 | — |
| Ethyl cellulose (ETHOCEL ™ 45 cp) | — | 74.90 |
| Triethyl citrate | 5.60 | 7.50 |
| Talc | 5.60 | 7.50 |

Solvent* ethanol:water (90:10)

| | | |
|---|---|---|
| Total Weight | 292.27 | 314.77 |

*Removed during process

Pellets 2 and 3 contain pyridostigmine bromide granules as pellet core and a pyridostigmine bromide drug layer over the pellet core. Pellet 2 contains 30 wt % functional coat, of the seal coated pellet core, and Pellet 3 contains 40 wt % functional coat, of the seal coated pellet core. Pellets 2 and 3 were made according to the following general procedure.

### Manufacturing Procedure:

### A. Drug Layering

1. Pyridostigmine bromide and METHOCEL™ E5 Premium LV were added to a mixture of ethanol and water (85:15) and mixed for not less than about 60 minutes to obtain a solution.
2. To the solution from step #1, talc was added and mixed for not less than about 30 minutes to obtain a uniform dispersion.
3. Pyridostigmine bromide granules were coated using a Wurster fluid bed coater, with an inlet air temperature of about 30-50° C., and sufficient air volume for fluidization.
4. When the product temperature reached 30° C., the dispersion from step #3 was sprayed onto the pyridostigmine bromide granules while maintaining the product temperature of 25-35° C. and sufficient air volume for the fluidization, until the target coating weight gain was achieved.

### B. Seal Coating

1. Hydroxypropyl cellulose was added to acetone in a stainless steel container and mixed to form a uniform solution.
2. To the solution from step #1, the purified water was added and mixed until a clear solution was obtained.
3. To the solution from step #2, talc was added and mixed for not less than about 30 minutes to form a homogenous dispersion.
4. Pyridostigmine granules were coated using a Wurster fluid bed coater with an inlet air temperature of 30-50° C., and sufficient air volume for fluidization. When the product temperature reached 30° C., the dispersion from step #3 was sprayed onto the granules while maintaining the product temperature of 28-30° C. and sufficient air volume for the fluidization, until the target coating weight gain was achieved.

### C. Functional Coating

1. ETHOCEL™ 20 cp/ETHOCEL™ 45 cp were added to dehydrated alcohol in a stainless steel container and mixed for not less than about 60 minutes to obtain a uniform dispersion.
2. To the dispersion from step #1, water was added and mixed for not less than about 30 minutes to obtain a homogeneous dispersion.
3. To the dispersion from step #2, TEC was added and mixed for not less than about 45 minutes.
4. To the dispersion from step #3, talc was added and mixed for not less than about 15 minutes to obtain a uniform dispersion.
5. Seal coated pyridostigmine granules (procedure B) were taken in a Wurster chamber and coated with the dispersion from step #4, until target coating weight gain was achieved.

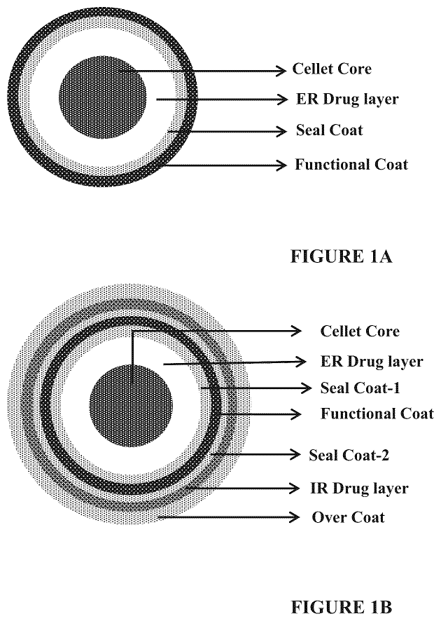

FIG. 1 depicts a schematic representation of pyridostigmine pellets, with and without an immediate release drug layer.

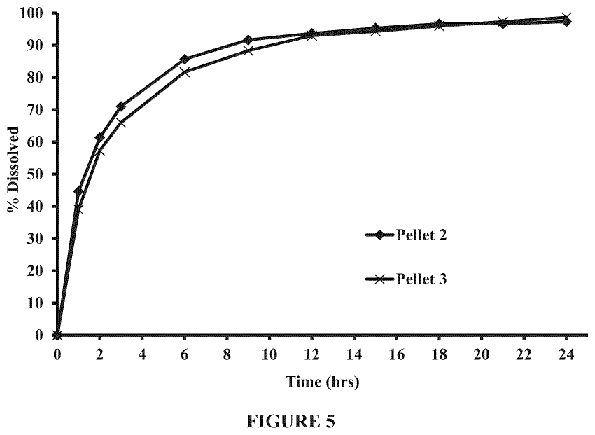

FIG. 5 compares dissolution profiles of pyridostigmine bromide Pellets 2 and 3, using USP Apparatus II, in 200 ml of 50 mM pH 6.8 phosphate buffer, at about 50 rpm and about 37° C.

FIG. 5 demonstrates that pellets containing pyridostigmine bromide granules as pellet core provide fast drug release, irrespective of their functional coat weight gain.

### Example 5: Pyridostigmine Bromide Pellet Composition Comprising Cellet Core

The present Example provides for pyridostigmine bromide pellet compositions comprising cellet cores. Eight different pellets, as outlined in Tables 6, 7, and 8, were prepared.

TABLE 6
Formulation of Pyridostigmine Bromide Pellets

| | Pellet 4 | Pellet 5 | Pellet 6 |
|---|---|---|---|
| Ingredients | mg/dose | mg/dose | mg/dose |
| Cellet core (350 μm) | 100.00 | 100.00 | 100.00 |

Drug Layer

| | | | |
|---|---|---|---|
| Pyridostigmine bromide | 150.00 | 150.00 | 150.00 |
| ETHOCEL ® Standard 20 Premium | 30.00 | 30.00 | 30.00 |
| Dibutyl Sebacate (DBS) | 3.00 | 3.00 | 3.00 |
| Talc | 6.00 | 6.00 | 6.00 |

Solvent* ethanol:water (90:10)
Seal coat

| | | | |
|---|---|---|---|
| METHOCEL ™ E5 Premium LV | 72.05 | 72.05 | 72.05 |
| Talc | 14.41 | 14.41 | 14.41 |
| Solvent* acetone:water (95:5) | NA | NA | NA |

Functional coat

| | | | |
|---|---|---|---|
| ETHOCEL ® Standard 20 Premium | 44.90 | 112.2 | — |
| Dibutyl sebacate | 9.00 | 22.40 | — |
| Talc | 6.70 | 16.86 | — |
| CAB-O-SIL ® | 1.10 | 2.80 | — |
| Cellulose acetate (CA-398-10NF/EP) | — | — | 89.70 |
| PEG 3350 | — | — | 17.90 |
| METHOCEL ™ E5 Premium LV | — | — | 13.50 |
| Solvent* ethanol:water (90:10) | — | — | — |
| Total Weight | 437.16 | 529.72 | 496.56 |

*Removed during process

TABLE 7
Formulation of Pyridostigmine Bromide Pellets

| | Pellet 7 | Pellet 8 |
|---|---|---|
| Ingredients | mg/dose | mg/dose |
| Cellet core (350 μm) | 100.00 | 100.00 |

Drug Layer

| | | |
|---|---|---|
| Pyridostigmine bromide | 150.00 | 150.00 |
| ETHOCEL ® Standard 20 Premium | 30.00 | 30.00 |
| Dibutyl sebacate (DBS) | 3.00 | 3.00 |
| Talc | 6.00 | 6.00 |

Solvent* ethanol:water (90:10) I.
Seal coat

| | | |
|---|---|---|
| METHOCEL ™ E5 Premium LV | 16.20 | 16.20 |
| Talc | 3.20 | 3.20 |

Solvent* acetone:water (95:5) I.
Functional coat

| | | |
|---|---|---|
| ETHOCEL ® Standard 20 Premium | 52.30 | 83.60 |
| Dibutyl sebacate | 10.50 | 16.80 |
| Talc | 7.80 | 12.50 |
| METHOCEL ™ E5 Premium LV | 5.20 | 8.40 |
| CAB-O-SIL ® | 1.30 | 3.10 |

Solvent* Acetone:Water (90:10)

| | | |
|---|---|---|
| Total Weight | 385.50 | 432.80 |

*Removed during process

TABLE 8
Formulation of Pyridostigmine Bromide Pellets

| | Pellet 9 | Pellet 10 | Pellet 11 |
|---|---|---|---|
| Ingredients | mg/dose | mg/dose | mg/dose |
| Cellet core (350/500 μm) | 100.0 | 100.0 | 100.0 |

Drug Layer

| | | | |
|---|---|---|---|
| Pyridostigmine bromide | 180.0 | 180.0 | 180.0 |
| ETHOCEL ® Standard 20 Premium | 36.00 | 36.00 | 36.00 |
| Talc | 7.20 | 7.20 | 7.20 |
| Dibutyl Sebacate (DBS) | 3.60 | 3.60 | 3.60 |

Solvent* ethanol:water (90:10) I.
Seal Coat

| | | | |
|---|---|---|---|
| METHOCEL ™ E5 Premium LV | 19.06 | 19.06 | 19.06 |
| Talc | 3.82 | 3.82 | 3.82 |

Solvent* acetone:water (95:5) I.
Functional Coat

| | | | |
|---|---|---|---|
| ETHOCEL ® Standard 20 Premium | 56.20 | 74.94 | 99.91 |
| Dibutyl Sebacate | 11.24 | 14.99 | 19.98 |
| Talc | 8.43 | 11.24 | 14.98 |
| METHOCEL ™ E5 Premium LV | 1.40 | 1.87 | 2.50 |
| CAB-O-SIL ® | 1.40 | 1.87 | 2.50 |

Solvent* ethanol:water (90:10) I.

| | | | |
|---|---|---|---|
| Total Weight | 428.35 | 454.59 | 489.55 |

*Removed during process

Pellets 4-11 contain a cellet core coated with a drug layer containing pyridostigmine bromide and a functional coat over the drug layer; Pellets 4 and 5 contain a functional coat comprising ETHOCEL® Standard 20 Premium, dibutyl sebacate, talc, and CAB-O-SIL®; Pellet 6 contains a cellet core and a functional coat comprising cellulose acetate 398, polyethylene glycol, and METHOCEL™ E5 Premium LV; and Pellets 7-11 contain functional coat comprising ETHOCEL® Standard 20 Premium, dibutyl sebacate, talc, METHOCEL™ E5 Premium LV, and CAB-O-SIL®. Pellets 4-11 were made according to the following general procedure.

### Manufacturing Procedure:

### A. Drug Layering

1. Pyridostigmine bromide and ETHOCEL® Standard 20 Premium were added to a mixture of ethanol and water (90:10) and mixed for not less than about 60 minutes to obtain a solution, followed by addition of dibutyl sebacate.
2. To the solution from step #1, talc was added and mixed for not less than about 30 minutes to obtain a uniform dispersion.
3. Cellet core was coated using a Wurster fluid bed coater, with an inlet air temperature of about 25-50° C., and sufficient air volume for fluidization. When the product temperature reached 30° C., the dispersion from step #2 was sprayed onto the cellets while maintaining the product temperature of 25-30° C. and sufficient air volume for the fluidization, until the target coating weight gain was achieved.

### B. Seal Coating

1. METHOCEL™ E5 Premium LV was added to a mixture of acetone and water (95:5) in a stainless steel container and mixed to form a uniform solution.
2. To the solution from step #2, talc was added and mixed for not less than about 30 minutes to obtain a homogeneous dispersion.
3. Pyridostigmine bromine drug-layered granules (procedure A) were coated using Wurster fluid bed coater with an inlet air temperature of 30-50° C., and sufficient air volume for fluidization. When the product temperature reached 30° C., the dispersion from step #2 was sprayed onto the drug-layered granules while maintaining the product temperature of about 28° C. and sufficient air volume for the fluidization, until the target coating weight gain was achieved.

### C. Functional Coating

1. ETHOCEL® Standard 20 Premium or cellulose acetate 398 (as per Pellets 4-11) was added to dehydrated alcohol and water or acetone and water mixture in a stainless steel container and mixed for not less than about 60 minutes to obtain a uniform solution.
2. To the solution from step #1, METHOCEL™ E5 Premium LV, and DBS/PEG were added and mixed until a clear solution was formed.
3. To the dispersion from step #2, talc and CAB-O-SIL® were added, and mixed for not less than about 30 minutes to obtain a uniform dispersion.
4. Seal coated pyridostigmine pellets (Step B) were taken in a Wurster chamber and coated with the dispersion from step #3, until target coating weight gain was achieved.

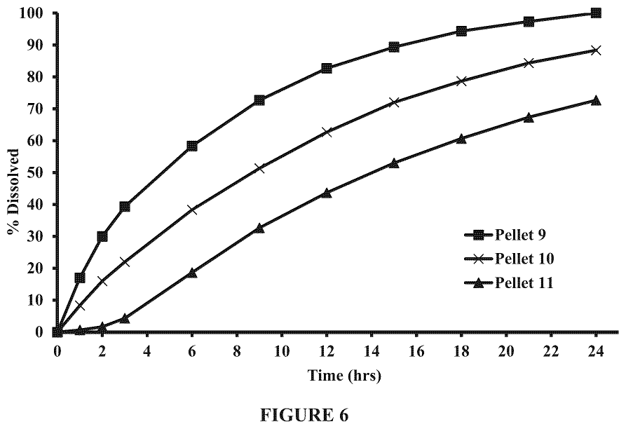

FIG. 6 compares dissolution profiles of pyridostigmine bromide Pellets 9-11, using USP Apparatus II (Paddle), at about 50 rpm and about 37° C., in 200 ml of 50 mM of pH 6.8 phosphate buffer.

FIG. 6 demonstrates that Pellets 10 and 11, containing higher functional coat weight gain, provide better controlled release of pyridostigmine bromide for a period of about 22 hours.

### Example 6: Effect of the Presence of Orifice in Functional Coat on Release Rate of Gastroretentive Pyridostigmine Compositions

The present Example provides for comparison of dissolution profiles of tablets comprising pyridostigmine bromide. Three different tablets were prepared as outlined in Table 9. Tablets 10 were made with and without an orifice in the functional coat to evaluate the effect an orifice has on dissolution profiles.

TABLE 9
Formulation of Pyridostigmine Bromide Gastroretentive Tablets

| | | | |
|---|---|---|---|
| | Tablet 8 | Tablet 13 | Tablet 14 |
| Ingredients | (mg/dose) | (mg/dose) | (mg/dose) |

Tablet Core

| | | | |
|---|---|---|---|
| Pyridostigmine bromide | 180.0 | 135.0 | 135.0 |
| Succinic acid | 50.0 | 80.0 | 80.0 |
| Sodium bicarbonate | 50.0 | 55.0 | 55.0 |
| Calcium carbonate | 125.0 | 65.0 | 65.0 |
| Crospovidone | 100.0 | 100.0 | 100.0 |
| Mannitol | 233.0 | 253.0 | 253.0 |
| BENECEL ™ K4M PH DC | 200.0 | — | 150.0 |
| METHOCEL ™ K100 | — | 300.0 | 150.0 |
| Prem LVCR | | | |
| CAB-O-SIL ® | 4.00 | 4.00 | 4.00 |
| Magnesium stearate | 8.00 | 8.00 | 8.00 |
| Total Weight | 950.0 | 1000.0 | 1000.0 |

Seal Coat

| | | | |
|---|---|---|---|
| Hydroxypropyl | 33.33 | — | — |
| cellulose | | | |
| Talc | 3.33 | — | — |
| Triethyl citrate | 3.33 | — | — |
| Solvent* | I. | — | — |
| acetone:water (95:5) | | | |

Functional Coat

| | | | |
|---|---|---|---|
| EUDRAGIT ® RL PO | 148.15 | 148.15 | 148.15 |
| Triethyl citrate | 22.22 | 22.22 | 22.22 |
| Talc | 29.63 | 29.63 | 29.63 |
| Solvent* | — | — | — |
| acetone:water (95:5) | | | |

Over Coat

| | | | |
|---|---|---|---|
| OPADRY ® white | 15.0 | — | — |
| Solvent*Purified | —. | — | — |
| water, USP | | | |
| Total Weight | 1205.0 | 1200.0 | 1200.0 |

*Removed during process

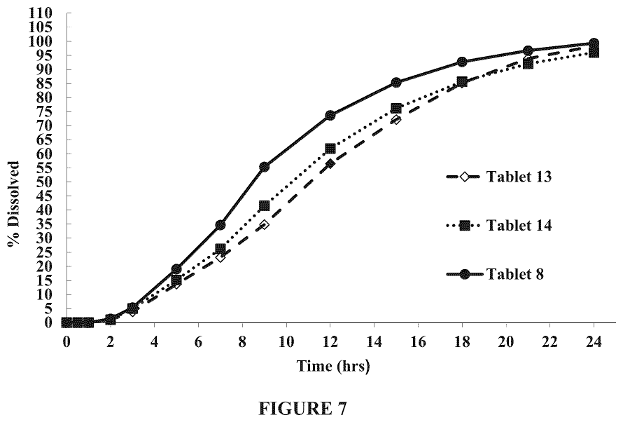

Tablet 8 contains 180 mg of pyridostigmine, 50 mg of succinic acid, 50 mg of sodium bicarbonate, 125 mg of calcium carbonate, and BENECEL™ K4M-DC. Tablets 13 and 14 contain 135 mg of pyridostigmine bromide, 80.0 mg of succinic acid, 55.0 mg of sodium bicarbonate, and 65.0 mg of calcium carbonate. Further, Tablet 13 contains METHOCEL™ K100 Prem LVCR and Tablet 14 contains a mixture of METHOCEL™ K100 Prem L VCR and BENECEL™ K4M-DC. Tablets 8, 13 and 14, each containing an orifice in fluid communication with the pull layer, were made according to the procedure as per Example 2, and without a seal coat step for Tablets 13 and 14. FIG. 7 compares dissolution profiles of Tablets 8, 13 and 14 in about 900 ml of 50 mM pH 5.0 acetate buffer containing 150 mM NaCl, using USP Apparatus I (Custom Basket), at 100 rpm and 37° C. FIG. 7 shows that Tablets 13 and 14 provide 10-15% slower drug release compared to Tablet 8.

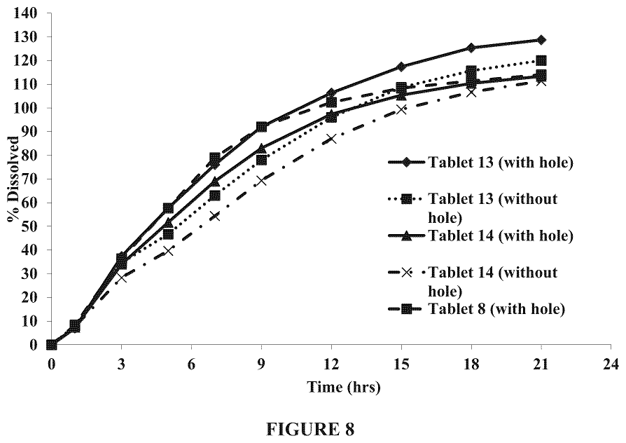

FIG. 8 compares dissolution profiles, of Tablets 13 and 14 containing an orifice/hole in the membrane/functional coat and Tablets 13 and 14 without orifice/hole in the membrane/functional coat. The dissolution testing was conducted in about 250 ml of 0.001 N HCl containing 100 mM NaCl, using USP Apparatus III (BIO-DIS), at 25 dpm and 37° C. FIG. 8 demonstrates that Tablets 13 and 14 without any orifice/hole in the functional coat provided reduced drug recovery compared to the Tablets 8, 13, and 14 containing an orifice/hole in the functional coat.

### Example 7: Effect of Coating Level of Functional Coat and Presence of Orifice/Hole in the Functional Coat on Release Rate of Gastroretentive Pyridostigmine Compositions

The present Example provides for comparison of dissolution profiles of tablets comprising pyridostigmine bromide and various functional coating compositions. Three different tablets, as outlined in Table 10, were prepared. The tablets were tested with and without an orifice/in their functional coat.

TABLE 10
Formulation of Pyridostigmine Bromide Tablets

| | | | |
|---|---|---|---|
| | Tablet 8 | Tablet 14 | Tablet 14A |
| Ingredients | (mg/dose) | (mg/dose) | (mg/dose) |

Tablet Core

| | | | |
|---|---|---|---|
| Pyridostigmine bromide | 180.0 | 135.0 | 135.0 |
| Succinic acid | 50.0 | 80.0 | 80.0 |
| Sodium bicarbonate | 50.0 | 55.0 | 55.0 |
| Calcium carbonate | 125.0 | 65.0 | 65.0 |
| Crospovidone | 100.0 | 100.0 | 100.0 |
| Mannitol (PARTECK ® M200) | 233.0 | 253.0 | 253.0 |
| BENECEL ™ K4M PH DC | 200.0 | — | 150.0 |
| METHOCEL ™ K100 | — | 300.0 | 150.0 |
| Prem LVCR | | | |
| CAB-O-SIL ® | 4.0 | 4.00 | 4.00 |
| Magnesium stearate | 8.0 | 8.00 | 8.00 |
| Total Weight | 950.0 | 1000.0 | 1000.0 |

Seal Coat

| | | | |
|---|---|---|---|
| Hydroxypropyl | 33.33 | — | — |
| Cellulose | | | |
| Talc | 3.33 | — | — |
| Triethyl citrate | 3.33 | — | — |
| Solvent* | I. | — | — |
| acetone:water (95:5) | | | |

Functional Coat

| | | | |
|---|---|---|---|
| EUDRAGIT ® RL PO | 148.15 | 148.15 | 185.18 |
| Triethyl citrate | 22.22 | 22.22 | 27.77 |
| Talc | 29.63 | 29.63 | 37.03 |
| Solvent* | — | — | — |
| acetone:water (95:5) | | | |
| Total Weight | 1190.0 | 1200.0 | 1250.0 |

*Removed during process

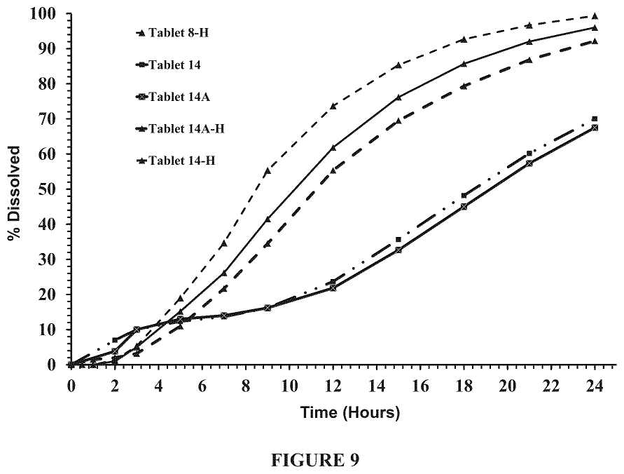

Tablets 8 and 14 contain 200 mg coating weight gain of the functional coat and Tablet 14A contains 250 mg coating weight gain of the functional coat. Tablets 8, 14, and 14A were made according to the procedure as per Example 2 (and without a seal coat step for Tablets 14 and 14A). FIG. 9 compares dissolution profiles, of Tablets 8, 14, and 14A containing an orifice/hole in the functional coat and Tablets 14 and 14A without orifice/hole in the functional coat. The dissolution testing was conducted in about 900 ml of 50 Mm pH 5.0 acetate buffer containing 150 mM NaCl, using USP Apparatus I (Custom Basket), at 100 rpm and 37° C. FIG. 9 demonstrates that coating weight gain has no significant effect on release rate of the tablets. The figure further demonstrates that tablets with orifice/hole provided higher release rate compared to tablets without orifice/hole.

### Example 8: Effect of Coating Level of Functional Coat and Presence of an Orifice/Hole in the Functional Coat on Floating Lag Time and Volume Expansion of Gastroretentive Pyridostigmine Compositions

The present example provides for evaluation of floating lag time and volume expansion of various tablets comprising pyridostigmine bromide. Eight different tablets, as outlined in Tables 11 and 12, were prepared with various levels of functional coating. The tablets were tested with and without an orifice in their functional coats.

TABLE 11
Formulation of Pyridostigmine Bromide Tablets

| | | | | |
|---|---|---|---|---|
| | Tablet 8 | Tablet 8A | Tablet 11 | Tablet 11A |
| Ingredients | (mg/dose) | (mg/dose) | (mg/dose) | (mg/dose) |

Tablet Core

| | | | | |
|---|---|---|---|---|
| Pyridostigmine | 180.0 | 180.0 | 135.0 | 135.0 |
| bromide | | | | |
| Succinic acid | 50.0 | 50.0 | 80.0 | 80.0 |
| Sodium bicarbonate | 50.0 | 50.0 | 55.0 | 55.0 |
| Calcium carbonate | 125.0 | 125.0 | 65.0 | 65.0 |
| Crospovidone | 100.0 | 100.0 | 200.0 | 200.0 |
| Mannitol | 233.0 | 233.0 | 153.0 | 253.0 |
| BENECEL ™ K4M | 200.0 | 200.0 | — | 150.0 |
| PH DC | | | | |
| METHOCEL ™ | — | — | 300.0 | 150.0 |
| K100 PREM LVCR | | | | |
| CAB-O-SIL ® | 4.00 | 4.00 | 4.00 | 4.00 |
| Magnesium stearate | 8.00 | 8.00 | 8.00 | 8.00 |
| Total Weight | 950.0 | 950.0 | 1000.0 | 1000.0 |

Seal Coat

| | | | | |
|---|---|---|---|---|
| Hydroxypropyl | 33.33 | 33.33 | — | — |
| cellulose | | | | |
| Talc | 3.33 | 3.33 | — | — |
| Triethyl citrate | 3.33 | 3.33 | — | — |
| Solvent* | — | — | — | — |
| acetone:water (95:5) | | | | |

Functional Coat

| | | | | |
|---|---|---|---|---|
| EUDRAGIT ® RL | 148.15 | 185.18 | 148.15 | 185.18 |
| PO | | | | |
| Triethyl citrate | 22.22 | 27.77 | 22.22 | 27.77 |
| Talc | 29.63 | 37.03 | 29.63 | 37.03 |
| Solvent* | — | — | — | — |
| acetone:water (95:5) | | | | |

Over Coat

| | | | | |
|---|---|---|---|---|
| Opadry white | 15.0 | 15.0 | — | — |
| Total Weight | 1205.0 | 1255.0 | 1200.0 | 1250.0 |

*Removed during process

TABLE 12
Formulation of Pyridostigmine Bromide Tablets

| | | | | |
|---|---|---|---|---|
| | Tablet 13 | Tablet 13A | Tablet 15 | Tablet 15A |
| Ingredients | (mg/dose) | (mg/dose) | (mg/dose) | (mg/dose) |

Tablet Core

| | | | | |
|---|---|---|---|---|
| Pyridostigmine | 135.0.0 | 135.0 | 135.0 | 135.0 |
| bromide | | | | |
| Succinic acid | 80.0 | 80.0 | 125.0 | 125.0 |
| Sodium bicarbonate | 55.0 | 55.0 | 75.0 | 75.0 |
| Calcium carbonate | 65.0 | 65.0 | 100.0 | 100.0 |
| Crospovidone | 100.0 | 100.0 | 200.0 | 200.0 |
| Mannitol | 253.0 | 253.0 | 153.0 | 153.0 |
| BENECEL ™ K4M | — | — | 100.0 | 100.0 |
| PH DC | | | | |
| METHOCEL ™ | 300.0 | 300.0 | 100.0 | 100.0 |
| K100 Prem LVCR | | | | |
| CAB-O-SIL ® | 4.00 | 4.00 | 4.00 | 4.00 |
| Magnesium stearate | 8.00 | 8.00 | 8.00 | 8.00 |
| Total Weight | 1000.0 | 1000.0 | 1000.0 | 1000.0 |

Functional Coat

| | | | | |
|---|---|---|---|---|
| EUDRAGIT ® RL | 148.15 | 185.18 | 148.15 | 185.18 |
| PO | | | | |
| Triethyl citrate | 22.22 | 27.77 | 22.22 | 27.77 |
| Talc | 29.63 | 37.03 | 29.63 | 37.03 |
| Solvent* acetone:water (95:5) | — | — | — | — |
| Total Weight | 1200.0 | 1250.0 | 1200.0 | 1250.0 |

*Removed during process

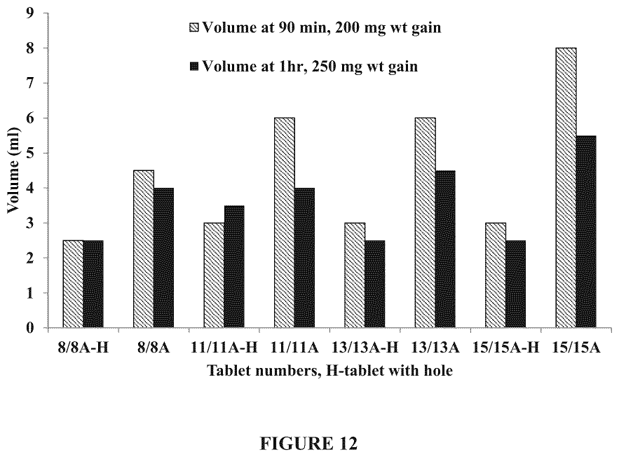

Tablets 8 and 8A contain 180 mg of pyridostigmine bromide, 50 mg of succinic acid, 50 mg of sodium bicarbonate, 125 mg of calcium carbonate, and a seal coat. Tablets 11 and 11A contain 135 mg of pyridostigmine bromide, 80 mg of succinic acid, 55 mg of sodium bicarbonate, and 65 mg of calcium carbonate. Tablets 13 and 13A contain 135 mg of pyridostigmine bromide, 80 mg of succinic acid, 55 mg of sodium bicarbonate, and 65 mg of calcium carbonate. Tablets 15 and 15A contain 135 mg of pyridostigmine bromide, 125 mg of succinic acid, 75 mg of sodium bicarbonate, and 100 mg of calcium carbonate. Tablets 8/8A and Tablets 13/13A contain 100 mg of crospovidone, and Tablets 11/11A and tablets 15/15A contain 200 mg of crospovidone. Tablets 8, 8A, 11, 11A, 13, 13A, 15, and 15A were made according to the procedure as per Example 2 (and without a seal coat step for Tablets 11, 11A, 13, 13A, 15, and 15A). FIG. 10 compares floating lag time of Tablets 8, 11, 13, and 15, with and without orifice/hole, at 200 mg functional coating weight gain, and Tablets 8A, 11A, 13A, and 15A, with and without orifice/hole, at 250 mg functional coating weight gain. The flotation studies were performed, using rotating bottle method at 5 rpm and 37° C., in 200 ml of 50 mM pH 4.5 acetate buffer containing 100 mM NaCl. The figure demonstrates that tablets with 200 mg functional coating weight gain exhibit shorter lag time compared to tablets with 250 mg functional coating weight gain. The figure further demonstrates that Tablets 8/8A containing a seal coat exhibit longer floating lag time compared to tablets without a seal coat (Tablets 11/11A, 13/13A, and 15/15A). FIG. 11 compares volumetric expansion at flotation of Tablets 8, 11, 13, and 15, with and without orifice/hole, at 200 mg functional coating weight gain, and Tablets 8A, 11A, 13A, and 15A, with and without orifice/hole, at 250 mg functional coating weight gain. The volume expansion studies were performed, using rotating bottle method at 5 rpm and 37° C., in 200 ml of 50 mM pH 4.5 acetate buffer containing 10 mM of NaCl. The figure demonstrates that tablets without orifice/hole exhibit higher volume expansion compared to tablets with orifice/hole. FIG. 12 compares volumetric expansion, at 90 minutes and at one hour, of Tablets 8, 11, 13, and 15, with and without orifice/hole, at 200 mg functional coating weight gain, and Tablets 8A, 11A, 13A, and 15A, with and without orifice/hole, at 250 mg functional coating weight gain. The volume expansion studies were performed, using rotating bottle method, at 5 rpm and 37° C., in 200 ml of 50 mM pH 4.5 acetate buffer containing 10 mM of NaCl. The figure demonstrates that tablets without orifice/hole exhibit higher volume expansion compared to tablets with orifice/hole. The figure further demonstrates that the difference in volume expansion between tablets with and without a hole is more prominent in tablets with 200 mg coating weight gain (Tablets 8, 11, 13, 15) compared to tablets with 250 mg coating weight gain (Tablets 8A, 11A, 13A, 15A).

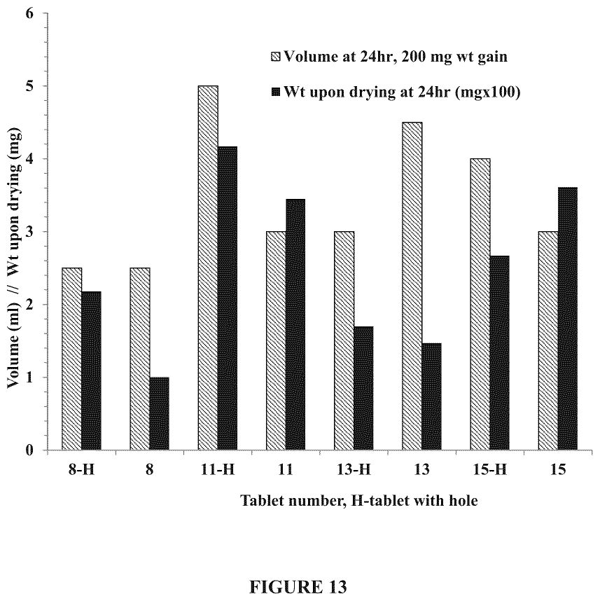

FIG. 13 compares volumetric expansion and weight gain, at 24 hours, of Tablets 8, 11, 13, and 15, with orifice/hole and without orifice/hole, at 200 mg functional coating weight gain. The volume expansion and weight gain studies were performed, using rotating bottle method at 5 rpm and 37° C., in 200 ml of 50 mM pH 4.5 acetate buffer containing 100 mM of NaCl. FIG. 13 demonstrates that tablets containing 200 mg of crospovidone (e.g., Tablets 11/11-H and 15/15-H) exhibit higher weight upon drying compared with tablets containing 100 mg of crospovidone (e.g., Tablets 8/8-H and 13/13-H).

### Example 9: Dissolution Profiles of Gastroretentive Pyridostigmine Compositions Using BIO-DIS Method

The present Example provides for measurements of dissolution profiles of various gastroretentive pyridostigmine compositions. Five compositions, as outlined in Table 13, were prepared, and tested using BIO-DIS method.

TABLE 13
Formulation of Pyridostigmine Bromide Tablets

| | | | | | |
|---|---|---|---|---|---|
| | Tablet 8 | Tablet 8B | Tablet 15 | Tablet 16 | Tablet 17 |
| Ingredients | (mg/dose) | (mg/dose) | (mg/dose) | (mg/dose) | (mg/dose) |

Tablet Core

| | | | | | |
|---|---|---|---|---|---|
| Pyridostigmine | 180.0 | 180.0 | 135.0 | 135.0 | 135.0 |
| bromide | | | | | |
| Succinic acid | 50.0 | 50.0 | 125.0 | 125.0 | 85.0 |
| Sodium | 50.0 | 50.0 | 75.0 | 75.0 | 56.0 |
| bicarbonate | | | | | |
| Calcium | 125.0 | 125.0 | 100.0 | 100.0 | 67.0 |
| carbonate | | | | | |
| Crospovidone | 100.0 | 100.0 | 200.0 | 200.0 | 200.0 |
| Mannitol | 233.0 | 233.0 | 153.0 | 153.0 | 45.0 |
| BENECEL ™ | 200.0 | 200.0 | 100.0 | — | — |
| K4M PH DC | | | | | |
| METHOCEL ™ | — | — | 100.0 | 200.0 | 400.0 |
| K100 Prem | | | | | |
| LVCR | | | | | |
| CAB-O-SIL ® | 4.00 | 4.00 | 4.00 | 4.00 | 4.00 |
| Magnesium | 8.00 | 8.00 | 8.00 | 8.00 | 8.00 |
| stearate | | | | | |
| Total Weight | 950.0 | 950.0 | 1000.0 | 1000.0 | 1000.0 |

Seal Coat

| | | | | | |
|---|---|---|---|---|---|
| Hydroxypropyl | 33.33 | 33.33 | — | — | — |
| cellulose | | | | | |
| Talc | 3.33 | 3.33 | — | — | — |
| Triethyl citrate | 3.33 | 3.33 | — | — | — |
| Solvent* | —. | — | — | — | — |
| acetone:water | | | | | |
| (95:5) | | | | | |

Functional Coat

| | | | | | |
|---|---|---|---|---|---|
| EUDRAGIT ® RL | 148.15 | 296.3 | 148.15 | 148.15 | 148.15 |
| PO | | | | | |
| Triethyl citrate | 22.22 | 44.44 | 22.22 | 22.22 | 22.22 |
| Talc | 29.63 | 59.26 | 29.63 | 29.63 | 29.63 |
| Solvent* | — | — | — | — | — |
| acetone:water | | | | | |
| (95:5) | | | | | |

Over Coat

| | | | | | |
|---|---|---|---|---|---|
| OPADRY ® white | 15.0 | 15.0 | — | — | — |
| Solvent*Purified | —. | — | — | — | — |
| water, USP | | | | | |
| Total Weight | 1205.0 | 1405.0 | 1200.0 | 1200.0 | 1200.0 |

*Removed during process

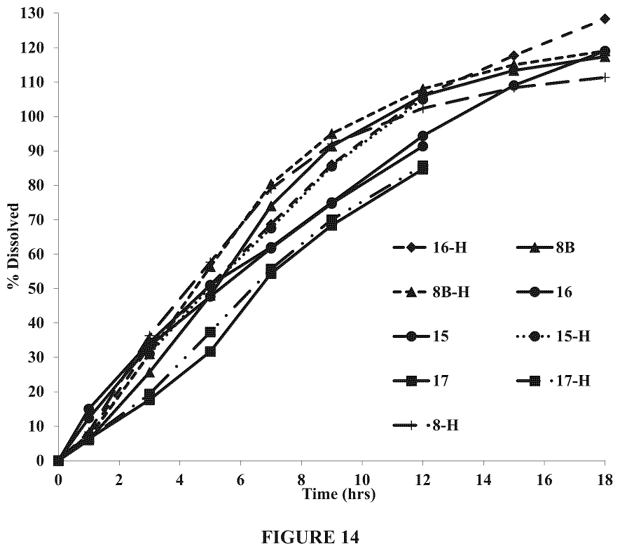

Tablets 8, 15, 16, and 17 contain 200 mg functional coating weight gain and Tablet 8B contains 400 mg functional coating weight gain. Tablets 8, 8B, 15, 16, and 17 were made according to the procedure as per Example 2 (and without the seal coat step for Tablets 15-17). FIG. 14 compares dissolution profiles of Tablets 8B, 15, 16, and 17 without an orifice/hole and Tablets 8, 8B, 15, 16, and 17 with an orifice/hole. Dissolution studies were performed using BIO-DIS method at 20 dpm and 37° C., in 250 ml of 0.001 N HCl containing 100 mM NaCl. FIG. 14 demonstrates that tablets without an orifice/hole exhibit slower drug release compared to tablets with an orifice/hole.

### Example 10: Dissolution Profiles of Gastroretentive Pyridostigmine Compositions Using USP-I Method

The present Example provides for measurements of dissolution profiles of various gastroretentive pyridostigmine compositions. Three compositions were prepared as outlined in Table 14 and tested using USP-I method.

TABLE 14
Formulation of Pyridostigmine Bromide Tablets

| | | | |
|---|---|---|---|
| | Tablet 8 | Tablet 18 | Tablet 19 |
| Ingredients | (mg/dose) | (mg/dose) | (mg/dose) |

Tablet Core

| | | | |
|---|---|---|---|
| Pyridostigmine bromide | 180.0 | 180.0 | 180.0 |
| Succinic acid | 50.0 | 125.0 | 125.0 |
| Sodium bicarbonate | 50.0 | 75.0 | 75.0 |
| Calcium carbonate | 125.0 | 100.0 | 100.0 |
| Crospovidone | 100.0 | 200.0 | 200.0 |
| Mannitol | 233.0 | 108.0 | 108.0 |
| BENECEL ™ K4M PH DC | 200.0 | — | 100.0 |
| METHOCEL ™ K100 | — | 200.0 | 100.0 |
| Premium LVCR | | | |
| CAB-O-SIL ® | 4.00 | 4.00 | 4.00 |
| Magnesium stearate | 8.00 | 8.00 | 8.00 |
| Total Weight | 950.0 | 1000.0 | 1000.0 |

Seal Coat

| | | | |
|---|---|---|---|
| Hydroxypropyl cellulose | 33.33 | — | — |
| Talc | 3.33 | — | — |
| Triethyl citrate | 3.33 | — | — |
| Solvent* | — | —. | — |
| acetone:water (95:5) | | | |

Functional Coat

| | | | |
|---|---|---|---|
| EUDRAGIT ® RL PO | 148.15 | 107.15 | 107.15 |
| Triethyl citrate | 22.22 | 21.42 | 21.42 |
| Talc | 29.63 | 21.42 | 21.42 |
| Solvent* | — | — | — |
| acetone:water (95:5) | | | |

Over Coat

| | | | |
|---|---|---|---|
| OPADRY ® white | 15.0 | — | — |
| Solvent*Purified | — | — | — |
| water, USP | | | |
| Total Weight | 1205.0 | 1150.0 | 1150.0 |

*Removed during process

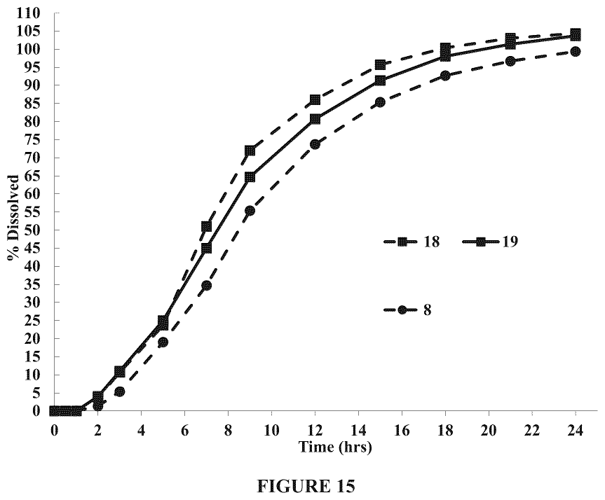

Tablet 8 contains 200 mg of BENECEL™ K4M PH DC and 100 mg of crospovidone, Tablet 18 contains 200 mg of METHOCEL™ and 200 mg of crospovidone, and Tablet 19 contains a mixture of 100 mg of METHOCEL™ K100 Premium LVCR, and 100 mg of BENECEL™ K4M PH DC, and 200 mg of crospovidone. Tablets 8, 18, and 19 were made according to the procedure as per Example 2 (and without a seal coat step for Tablets 18 and 19). Tablets 8, 18, and 19 were tested for dissolution in 900 ml of 50 mM pH 5.0 acetate buffer containing 150 mM NaCl, using USP Apparatus I (Custom Basket), at about 100 rpm and about 37° C. FIG. 15 demonstrates that tablets containing 200 mg of crospovidone (Tablets 18 and 19) exhibit faster drug release and better drug recovery compared to Tablet 8 containing 100 mg of crospovidone.

### Example 11: Dissolution Profiles of Gastroretentive Pyridostigmine Compositions Using USP-I Method

The present Example provides for measurements of dissolution profiles of various gastroretentive pyridostigmine compositions. Three compositions, as outlined in Table 15, were prepared and tested using USP-I method.

TABLE 15
Formulation of Pyridostigmine Bromide Tablets

| | | | |
|---|---|---|---|
| | Tablet 8 | Tablet 20 | Tablet 21 |
| Ingredients | (mg/dose) | (mg/dose) | (mg/dose) |

Tablet Core

| | | | |
|---|---|---|---|
| Pyridostigmine | 180.0 | 305.0 | 255.0 |
| bromide | | | |
| Succinic acid | 50.0 | 80.0 | 80.0 |
| Sodium bicarbonate | 50.0 | 55.0 | 55.0 |
| Calcium carbonate | 125.0 | 65.0 | 65.0 |
| Crospovidone | 100.0 | 100.0 | 100.0 |
| Mannitol (PARTECK ® M200) | 233.0 | 73.0.0 | 123.0 |
| BENECEL ™ K4M PH DC | 200.0 | 150.0 | 150.0 |
| METHOCEL ™ K100 | — | 150.0 | 150.0 |
| Prem LVCR | | | |
| CAB-O-SIL ® | 4.00 | 10.00 | 10.00 |
| Magnesium stearate | 8.00 | 12.00 | 12.00 |
| Total Weight | 950.0 | 1000.0 | 1000.0 |

Seal Coat

| | | | |
|---|---|---|---|
| Hydroxypropyl | 33.33 | — | — |
| cellulose | | | |
| Talc | 3.33 | — | — |
| Triethyl citrate | 3.33 | — | — |
| Solvent* | — | — | — |
| acetone:water (95:5) | | | |

Functional Coat

| | | | |
|---|---|---|---|
| EUDRAGIT ® RL PO | 148.15 | 148.15 | 148.15 |
| Triethyl citrate | 22.22 | 22.22 | 22.22 |
| Talc | 29.63 | 29.63 | 29.63 |
| Solvent* | — | — | — |
| acetone:water (95:5) | | | |

Over Coat

| | | | |
|---|---|---|---|
| OPADRY ® white | 15.0 | — | — |
| Solvent*Purified | — | — | — |
| water, USP | | | |
| Total Weight | 1205.0 | 1200.0 | 1200.0 |

*Removed during process

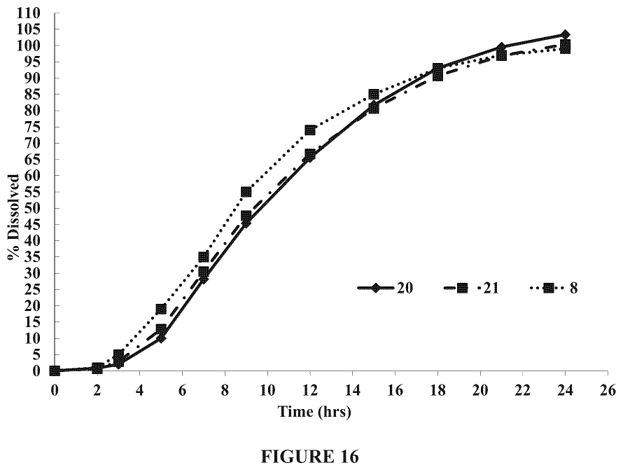

Tablet 8 contains 200 mg of BENECEL™, Tablets 20 and 21 contain 150 mg each of BENECEL™ and METHOCEL™. Tablets 8, 20, and 21 were made according to the procedure as per Example 2 (and without a seal coat step for Tablets 20 and 21). Tablets 8, 20, and 21 were tested for dissolution in about 900 ml of 50 mM pH 5.0 acetate buffer containing 150 mM NaCl, using USP Apparatus I (Custom Basket), at 100 rpm and 37° C. FIG. 16 demonstrates that tablets containing a mixture of 150 mg each of BENECEL™ and METHOCEL™ (Tablets 20 and 21) provide more controlled release compared to Tablet 8 containing 200 mg of BENECEL™.

### Example 12: Dissolution Profiles of Gastroretentive Pyridostigmine Compositions Using USP-I Method

The present Example provides for measurements of dissolution profiles of various gastroretentive pyridostigmine compositions. Three compositions were prepared as outlined in Table 15 and tested using USP-I method.

TABLE 16
Formulation of Pyridostigmine Bromide Tablets

| | | | |
|---|---|---|---|
| | Tablet 8 | Tablet 22 | Tablet23 |
| Ingredients | (mg/dose) | (mg/dose) | (mg/dose) |

Tablet Core

| | | | |
|---|---|---|---|
| Pyridostigmine | 180.0 | 135.0 | 135.0 |
| bromide | | | |
| Succinic acid | 50.0 | 80.0 | 80.0 |
| Sodium bicarbonate | 50.0 | 55.0 | 55.0 |
| Calcium carbonate | 125.0 | 65.0 | 65.0 |
| Crospovidone | 100.0 | 100.0 | 100.0 |
| Mannitol | 233.0 | 249.0 | 299.0 |
| Oxide Pigment | — | — | 12.0 |
| Black | | | |
| BENECEL ™ K4M | 200.0 | 150.0 | 150.0 |
| PH DC | | | |
| METHOCEL ™ K100 | — | 150.0 | 150.0 |
| Prem LVCR | | | |
| CAB-O-SIL ® | 4.00 | 10.00 | 10.00 |
| Magnesium | 8.00 | 12.00 | 12.00 |
| stearate | | | |
| Total Weight | 950.0 | 1006.0 | 1068.0 |

Seal Coat-1

| | | | |
|---|---|---|---|
| Hydroxypropyl | 33.33 | — | — |
| cellulose | | | |
| Talc | 3.33 | — | — |
| Triethyl citrate | 3.33 | — | — |
| Solvent* | — | — | — |
| acetone:water (95:5) | | | |

Functional Coat

| | | | |
|---|---|---|---|
| EUDRAGIT ® RL PO | 148.15 | 148.15 | 148.15 |
| Triethyl citrate | 22.22 | 22.22 | 22.22 |
| Talc | 29.63 | 29.63 | 29.63 |
| Solvent* | — | — | — |
| acetone:water (95:5) | | | |

Seal Coat-2

| | | | |
|---|---|---|---|
| OPADRY ® II, Clear | — | — | 10.0 |
| Solvent*Purified | — | — | I. |
| water, USP | | | |

Drug Layer

| | | | |
|---|---|---|---|
| Pyridostigmine | — | — | 45.0 |
| bromide | | | |
| Hydroxypropyl | — | — | 9.0 |
| cellulose | | | |
| Solvent*Dehydrated | — | — | — |
| alcohol, | | | |

Over Coat

| | | | |
|---|---|---|---|
| OPADRY ® white | 15.0 | — | 40.0 |
| Solvent*Purified | —. | — | —. |
| water, USP | | | |
| Total Weight | 1205.0 | 1206.0 | 1372.0 |

*Removed during process

Tablet 23 contains an immediate release drug layer. Tablet 8 contains 200 mg of BENECEL™ K4M PH DC, Tablets 22 and 23 contain 150 mg each of BENECEL™ K4M PH DC and METHOCEL™ K100 Prem LVCR. Tablets 8, 22, and 23 were made according to the procedure as per Example 2, with the following variations: Tablet 22 and Tablet 23 do not include a seal coat between the tablet core and the functional coat, and Tablet 23 was further coated with a seal coat (over the functional coat), an IR drug layer coat, and an over coat as follows:

### D. Seal Coat

1. OPADRY® Clear was added to purified water in a stainless steel container and mixed to form a uniform dispersion.
2. Tablet core 23 with a functional coat was seal coated with the dispersion from Step 1, using a perforated pan coater with an inlet air temperature of 25° C.-60° ° C. at a product temperature of 30-45° C.

### E. IR Drug Layer

1. Seal coated pyridostigmine bromide tablets from Step D were further coated with a solution of pyridostigmine bromide, hydroxypropyl cellulose in dehydrated alcohol, using a perforated pan coater with an inlet air temperature of 25° C.-60° C. at a product temperature of 30-45° C.

### F. Over Coat

1. Weighed quantity of OPADRY® white was added to a required amount of purified water and mixed to obtain a uniform dispersion.
2. The tablets with IR drug layer from Step E were further coated with the dispersion from step #1 in a perforated coating pan with inlet air temperature at 25° C.-45° C.
3. The coated tablets from step #2 were dried in the coating pan to a moisture content of below 1.5%.

Tablets 8, 22, and 23 were tested for dissolution in about 900 ml of 50 mM pH 5.0 acetate buffer containing 150 mM of NaCl, using USP Apparatus I (Custom Basket), at 100 rpm and 37° C. FIG. 17 demonstrates that the tablet containing an immediate release drug layer (Tablet 23) eliminates lag time compared to tablets that do not contain an immediate release drug layer (Tablets 8 and 22).

### Example 13: Additional Gastroretentive Pyridostigmine Compositions

The present Example provides for various gastroretentive pyridostigmine compositions. Ten different compositions were prepared as outlined in Tables 17 and 18.

TABLE 17
Formulation of Pyridostigmine Bromide Tablets

| | | | | | |
|---|---|---|---|---|---|
| | Tablet 24 | Tablet 25 | Tablet 26 | Tablet 27 | Tablet 28 |
| Ingredients | (mg/dose) | (mg/dose) | (mg/dose) | (mg/dose) | (mg/dose) |

Tablet Core

| | | | | | |
|---|---|---|---|---|---|
| Pyridostigmine | 305.0 | 305.0 | 305.0 | 255.0 | 255.0 |
| bromide | | | | | |
| Succinic acid | 80.0 | 80.0 | 80.0 | 80.0 | 80.0 |
| Sodium | 55.0 | 55.0 | 55.0 | 55.0 | 55.0 |
| bicarbonate | | | | | |
| Calcium carbonate | 65.0 | 65.0 | 65.0 | 65.0 | 65.0 |
| Crospovidone | 100.0 | 100.0 | 100.0 | 100.0 | 100.0 |
| Mannitol | 73.0 | — | — | 123.0 | — |
| BENECEL ™ K4M | 150.0 | 186.5.0 | 236.5 | 150.0 | 211.5 |
| PH DC | | | | | |
| METHOCEL ™ | 150.0 | 186.5 | 236.5 | 150.0 | 211.5 |
| K100 Prem LVCR | | | | | |
| CAB-O-SIL ® | 10.00 | 10.00 | 10.00 | 10.00 | 10.00 |
| Magnesium | 12.00 | 12.00 | 12.00 | 12.00 | 12.00 |
| stearate | | | | | |
| Total Weight | 1000.0 | 1000.0 | 1100.0 | 1000.0 | 1000.0 |

Functional Coat

| | | | | | |
|---|---|---|---|---|---|
| EUDRAGIT ® | 148.15 | 148.15 | 148.15 | 148.15 | 148.15 |
| RL PO | | | | | |
| Triethyl | 22.22 | 22.22 | 22.22 | 22.22 | 22.22 |
| citrate | | | | | |
| Talc | 29.63 | 29.63 | 29.63 | 29.63 | 29.63 |
| Solvent* | — | — | — | —. | — |
| acetone:water (95:5) | | | | | |
| Total Weight | 1200.0 | 1200.0 | 1300.0 | 1200.0 | 1200.0 |

TABLE 18
Formulation of Pyridostigmine Bromide Tablets

| | | | | | |
|---|---|---|---|---|---|
| | Tablet 29 | Tablet 30 | Tablet 31 | Tablet 32 | Tablet 33 |
| Ingredients | (mg/dose) | (mg/dose) | (mg/dose) | (mg/dose) | (mg/dose) |

Tablet Core

| | | | | | |
|---|---|---|---|---|---|
| Pyridostigmine | 70.0 | 155.0 | 205.0 | 305.0 | 100.0 |
| bromide | | | | | |
| Succinic acid | 80.0 | 80.0 | 80.0 | 80.0 | 80.0 |
| Sodium | 55.0 | 55.0 | 55.0 | 55.0 | 55.0 |
| bicarbonate | | | | | |
| Calcium | 65.0 | 65.0 | 65.0 | 65.0 | 65.0 |
| carbonate | | | | | |
| Crospovidone | 100.0 | 100.0 | 100.0 | 100.0 | 100.0 |
| Mannitol | 308.0 | 223.0 | 173.0 | — | 278.0 |
| BENECEL ™ | 150.0 | 150.0 | 150.0 | 150.0 | 150.0 |
| K4M PH DC | | | | | |
| METHOCEL ™ K100 | 150.0 | 150.5 | 150.0 | 223.0 | 150.0 |
| Prem LVCR | | | | | |
| CAB-O-SIL ® | 10.00 | 10.00 | 10.00 | 10.00 | 10.0 |
| Magnesium | 12.00 | 12.00 | 12.00 | 12.00 | 12.0 |
| stearate | | | | | |
| Total Weight | 1000.0 | 1000.5 | 1000.0 | 1000.0 | 1000.0 |

Functional Coat

| | | | | | |
|---|---|---|---|---|---|
| EUDRAGIT ® | 148.15 | 148.15 | 148.15 | 148.15 | 148.15 |
| RL PO | | | | | |
| Triethyl citrate | 22.22 | 22.22 | 22.22 | 22.22 | 22.22 |
| Talc | 29.63 | 29.63 | 29.63 | 29.63 | 29.63 |
| Solvent* | — | — | —. | — | — |
| acetone:water (95:5) | | | | | |
| Total Weight | 1200.0 | 1200.5 | 1200.0 | 1200.0 | 1200.0 |

*Removed during process

Tablets 24-26, and 32 contain 305 mg of pyridostigmine bromide; Tablets 27 and 28 contain 255 mg of pyridostigmine bromide, Tablet 29 contains 70 mg of pyridostigmine bromide, Tablet 30 contains 155 mg of pyridostigmine bromide, Tablet 31 contains 205 mg of pyridostigmine bromide, and Tablet 33 contains 100 mg of pyridostigmine bromide. Tablets 24, 27, 29-31, and Tablet 33 contain 150 mg each of BENECEL™ K4M PH DC and METHOCEL™ K100 Premium LVCR, Tablet 25 contains 186.5 mg each of BENECEL™ K4M PH DC and METHOCEL™ K100 Premium LVCR, Tablet 26 contains 236.5 mg each of BENECEL™ K4M PH DC and METHOCEL™ K100 Premium LVCR, Tablet 28 contains 211.5 mg each of BENECEL™ K4M PH DC and METHOCEL™ K100 Premium LVCR, and Tablet 32 contains 150.0 mg of BENECEL™ K4M PH DC and 223.0 mg of METHOCEL™ K100 Premium LVCR. Tablets 24-33 were made according to the procedure as per Example 2.

### Example 14: Additional Gastroretentive Pyridostigmine Compositions with IR Drug Layer

The present Example provides for gastroretentive pyridostigmine compositions that comprise an immediate release drug layer. Four different compositions were prepared as outlined in Table 19.

TABLE 19
Formulation of Pyridostigmine Bromide Tablets

| | | | | |
|---|---|---|---|---|
| | Tablet 34 (with | | | |
| Ingredients | orifice/hole) | Tablet 35 | Tablet 36 | Tablet 37 |

Tablet Core

| | | | | |
|---|---|---|---|---|
| Pyridostigmine | 135.0 | 135.0 | 70.00 | 160.0 |
| bromide | | | | |
| Succinic acid | 80.0 | 80.0 | 80.0 | 80.0 |
| Sodium bicarbonate | 55.0 | 55.0 | 55.0 | 55.0 |
| Calcium carbonate | 65.0 | 65.0 | 65.0 | 65.0 |
| Crospovidone | 100.0 | 100.0 | 100.0 | 100.0 |
| Mannitol | 235.5 | 231.0 | 278.0 | 218.0 |
| Oxide Pigment | 7.5 | 12.0 | 12.0 | 0 |
| Black | | | | |
| BENECEL ™ K4M | 150.0 | 150.0 | 150.0 | 150.0 |
| PH DC | | | | |
| METHOCEL ™ K100 | 150.0 | 150.0 | 150.0 | 150.0 |
| Prem LVCR | | | | |
| CAB-O-SIL ® | 10.00 | 10.00 | 10.0 | 10.0 |
| Magnesium stearate | 12.00 | 12.00 | 12.0 | 12.0 |
| Total Weight | 1000.0 | 1000.0 | 982.0 | 1000.0 |

Functional Coat

| | | | | |
|---|---|---|---|---|
| EUDRAGIT ® RL PO | 148.15 | 148.15 | 148.15 | 148.15 |
| Triethyl citrate | 22.22 | 22.22 | 22.22 | 22.22 |
| Talc | 29.63 | 29.63 | 29.63 | 29.63 |
| Solvent* | — | — | — | — |
| acetone:water (95:5) | | | | |

Seal Coat

| | | | | |
|---|---|---|---|---|
| OPADRY ® II, Clear | 10.0 | 10.0 | 10.0 | 10.0 |
| Solvent*Purified | — | — | —. | — |
| water, USP | | | | |

Drug Layer

| | | | | |
|---|---|---|---|---|
| Pyridostigmine | 45.0 | 45.0 | 30.0 | 20.0 |
| bromide | | | | |
| Hydroxypropyl | 9.0 | 9.0 | 9.0 | 4.0 |
| cellulose | | | | |
| Solvent*Dehydrated | — | — | — | —. |
| alcohol, USP | | | | |

Over Coat

| | | | | |
|---|---|---|---|---|
| OPADRY ® White | 40.0 | 40.0 | 40.0 | 40.0 |
| Solvent*Purified | —. | I. | —. | — |
| water, USP | | | | |
| Total Weight | 1304.0 | 1304.0 | 1271.0 | 1274.0 |

*Removed during process

Tablets 34 and 35 contain an immediate release drug layer containing 45 mg of pyridostigmine bromide and an extended release component/tablet core containing 135 mg of pyridostigmine bromide. Tablet 36 contains an immediate release drug layer containing 30 mg of pyridostigmine bromide and an extended release component/tablet core containing 70 mg of pyridostigmine bromide. Tablet 37 contains an immediate release drug layer containing 20 mg of pyridostigmine bromide and an extended release component/tablet core containing 160 mg of pyridostigmine bromide. Tablets 34-37 contain 150 mg each of BENECEL™ K4M PH DC and METHOCEL™ K100 Prem LVCR. Tablets 34, 36, and 37 contain a laser drilled hole in the functional coat and Tablet 35 is without a hole. Tablets 34, 35, and 37 were made as per Tablet 23 in Example 12. Tablet 36 is made as per Tablet 23 in Example 12.

### Example 15: Oral Bioavailability of Pyridostigmine for Tablet 34 (Gastroretentive Dosage Form with Hole)

A single dose pharmacokinetic (PK) study was conducted in healthy volunteers under fed conditions (under low fat-low calorie and high fat-high calorie conditions) to evaluate the PK performance of extended release compositions of the disclosure using Tablet 34. An open-label, balanced, nonrandomized, single-dose, two-treatment, one-way crossover, comparative bioavailability study was conducted in 15 normal, healthy, adult, human subjects under high-fat high-calorie (HF-HC) breakfast conditions and under low fat-low calorie (LF-LC) breakfast conditions.

Pharmacokinetic parameters for pyridostigmine are summarized in Table 20.

TABLE 20
Pharmacokinetics Results of Pyridostigmine

| | Mean ± SD (CV %) (N = 15) | |
|---|---|---|
| Pharmacokinetic | LF-LC | HF-HC |
| parameters (units) | (Condition I) | (Condition II) |
| Cₘₐₓ (ng/mL) | 42.178 ± 9.890 | 45.073 ± 6.094 |
| | (23.448) | (13.520) |
| AUC₀₋ₜ (ng · hr/mL) | 670.921 ± 287.971 | 735.1391 ± 173.317 |
| | (42.922) | (23.576) |
| AUC0-inf (ng · hr/mL) | 684.726 ± 292.086 | 749.674 ± 174.634 |
| | (42.657) | (23.295) |
| Tₘₐₓ (hr)* | 7.10 ± 3.80 | 11.0 ± 2.77 |
| | (53.55) | (25.19) |
| Kₑₗ (hr−1) | 0.15 ± 0.03 | 0.15 ± 0.03 |
| | (18.90) | (22.02) |
| t_{1/2} (hr) | 4.77 ± 1.03 | 4.70 ± 1.06 |
| | (21.65) | (22.52) |
| AUC Extrapolated | 2.209 ± 1.383 | 2.084 ± 1.657 |
| (%) | (62.610) | (79.549) |

The data from this study (Table 20/FIG. 20) demonstrates that Tablet 34 provides a therapeutic plasma concentration of pyridostigmine for at least about 22 hours.

### Example 16: Oral Bioavailability of Pyridostigmine for Tablet 35 (Gastroretentive Dosage Form without Hole)

A single dose pharmacokinetic (PK) study was conducted in healthy volunteers under fed conditions to evaluate the PK performance of extended release compositions of the disclosure using Tablet 35. An open-label, balanced, nonrandomized, single-dose, two-treatment, one-way crossover, comparative bioavailability study was conducted in 15 normal, healthy, adult, human subjects under high-fat high-calorie (HF-HC) breakfast conditions and under low fat-low calorie (LF-LC) breakfast conditions.

Pharmacokinetic parameters for pyridostigmine are summarized in Table 21.

TABLE 21
Pharmacokinetics Results of Pyridostigmine

| | Mean ± SD (CV %) (N = 15) | |
|---|---|---|
| Pharmacokinetic | LF-LC | HF-HC |
| parameters (units) | (Condition I) | (Condition II) |
| Cₘₐₓ (ng/mL) | 47.444 ± 12.070 | 43.204 ± 13.455 |
| | (25.440) | (31.144) |
| AUC₀₋ₜ (ng · hr/mL) | 628.282 ± 322.601 | 761.807 ± 297.513 |
| | (51.346) | (39.054) |
| AUC0-inf (ng · hr/mL) | 635.600 ± 336.928 | 643.820 ± 161.166 |
| | (53.009) | (25.033) |
| Tₘₐₓ (hr)* | 8.10 ± 6.25 | 15.44 ± 4.92 |
| | (77.14) | (31.85) |
| Kₑₗ (hr−1) | 0.14 ± 0.04 | 0.15 ± 0.05 |
| | (27.80) | (31.12) |
| t_{1/2} (hr) | 5.23 ± 1.61 | 5.20 ± 2.03 |
| | (30.77) | (39.15) |
| AUC Extrapolated | 2.654 ± 2.429 | 3.621 ± 4.396 |
| (%) | (91.528) | (121.392) |

The data from this study (Table 21/FIG. 21) demonstrate that Tablet 35 provides a therapeutic plasma concentration of pyridostigmine for at least about 22 hours.

### Example 17: Volume Expansion and Texture Analysis/Compressibility for Tablet 34 (Gastroretentive Dosage Form with Hole)

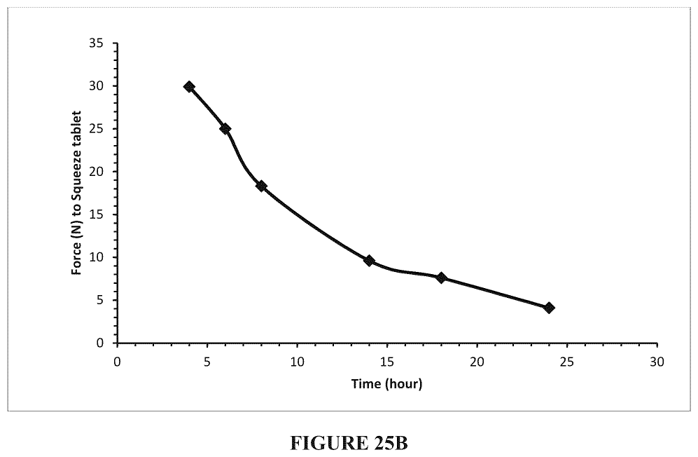

Tablet 34 was tested for volume expansion and texture analysis/compressibility. The volume expansion studies were performed using rotating bottle method, at 5 rpm and 37° C., in 200 ml of 0.001 N HCL containing 10 mM NaCl. FIG. 25A demonstrates that Tablet 34 exhibits 100% volume gain at about 30 minutes, 200% volume gain at about 1 hour, and 300% volume gain at 8 about hours post-administration of the tablet into the dissolution medium. Tablet 34 was simultaneously tested for its texture/compressibility at various time points using TA.XT^{Plus} apparatus. FIG. 25B demonstrates that the compression force required to squeeze out the matrix core at 2 hours post-administration, at about 200% volume gain, was 30 N; at 8 hours post-administration, at about 300% volume gain, was 18.3 N; and at 24 hours post-administration, at about 250% volume gain, was 4.1 N. It was observed that the tablet maintained its GRS attributes of floatation and expansion for at least about 14 hours, e.g., about 24 hours. The experiment demonstrates that Tablet 34, in its expanded state, e.g., between about 250% and about 300% volume gain, can withstand forces of about 10 N until 14 hours, and the matrix core can be subsequently squeezed thereafter even with force less than 5 N, after at least about 20 hours, e.g., about 24 hours, post-administration.

### Example 18: Oral Bioavailability of Pyridostigmine Under Medium Fat-Medium Calorie and High Fat-High Calorie Conditions

A single dose and multiple dose cross-over pharmacokinetic (PK) study was conducted in healthy volunteers under fed conditions (MF-MC and HF-HC conditions) to evaluate and compare PK performance of extended release compositions of the disclosure using Tablet 37, administered once-a-day, and marketed pyridostigmine bromide tablet, MESTINON® (60 mg), administered thrice-a-day. An open-label, randomized, two treatment, four period, four-arm, single dose and multiple dose, cross-over, bioavailability study was conducted in 16 normal, healthy, adult, human subjects under medium-fat-medium calorie (600-650 Kcal; ˜40% fat) and high-fat high-calorie (800-900 Kcal; ˜50% fat) breakfast conditions.

Pharmacokinetic parameters for pyridostigmine are summarized in Table 22.

TABLE 22
Pharmacokinetics Results (N = 16 subjects)

| | MF-MC | | HF-HC | |
|---|---|---|---|---|
| | 600-650 Kcal; ~40% fat | | 800-900 Kcal; ~50% fat | |
| | Tablet 37 | MESTINON ® | Tablet 37 | MESTINON ® |
| PK Parameters | (180 mg) | (60 mg × 3) | (180 mg) | (60 mg × 3) |
| AUC_{0-∞} (ng · | 668 | 877 | 853 | 906.5 |
| hr/mL) (% CV) | (51.5) | (30.7) | (26.5) | (30) |
| Cₘₐₓ (ng/mL) | 38.6 | 51.6 | 43.5 | 48.2 |
| (% CV) | (40.2) | (24.2) | (23.1) | (37) |

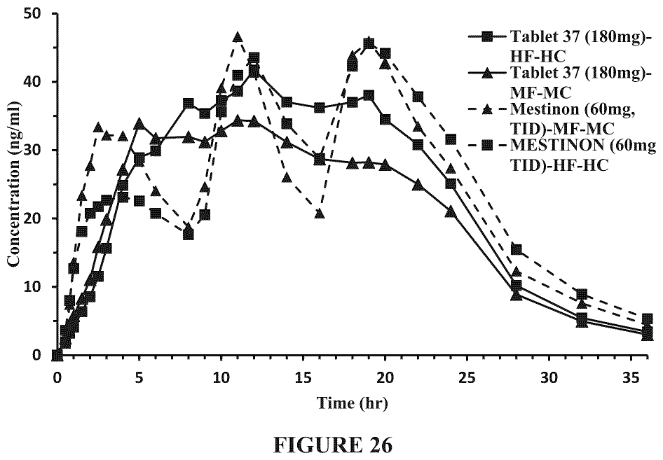

The data from this study (Table 22/FIG. 26) demonstrates that Tablet 37 provides a therapeutic plasma concentration of pyridostigmine for at least about 22 hours under MF-MC conditions and under HF-HC conditions. The data further indicates that the pyridostigmine composition of the disclosure (Tablet 37) exhibits minimal variability in PK profile, specially under HF-HC conditions, as compared to the marketed MESTINON® product (60 mg×3). The test (Tablet 37) and reference products (Mestinon® (60×3)) were well tolerated by the subjects. No serious adverse events were reported during the conduct of the study.

### Example 19: Comparison of Fluctuation Index of Pyridostigmine Compositions of the Disclosure and MESTINON® Under Medium Fat-Medium Calorie and High Fat-High Calorie Conditions

A single dose and multiple dose cross-over pharmacokinetic (PK) study was conducted in healthy volunteers under fed conditions (MF-MC and HF-HC conditions) to evaluate and compare PK performance of extended release compositions of the disclosure using Tablet 37, administered once-a-day, and marketed pyridostigmine bromide tablet, MESTINON® (60 mg), administered thrice-a-day. An open-label, randomized, two treatment, four period, four-arm, single dose and multiple dose, cross-over, bioavailability study was conducted in 16 normal, healthy, adult, human subjects under medium-fat-medium calorie (600-650 Kcal; ˜40% fat) and high-fat high-calorie (800-900 Kcal; ˜50% fat) breakfast conditions.

Pharmacokinetic parameters for pyridostigmine are summarized in Table 22 and 23.

TABLE 23
Pharmacokinetics Results (N = 16 subjects)

| | MF-MC | | HF-HC | |
|---|---|---|---|---|
| | 600-650 Kcal; ~40% fat | | 800-900 Kcal; ~50% fat | |
| | Tablet 37 | MESTINON ® | Tablet 37 | MESTINON ® |
| PK Parameters | (180 mg) | (60 mg × 3) | (180 mg) | (60 mg × 3) |
| Cₘₐₓ (ng/mL) | 38.568 | 51.593 | 43.528 | 48.212 |
| Cₘᵢₙ (ng/mL) | 21.08 | 18.75 | 25.09 | 17.64 |
| (% CV) | | | | |
| Cₐᵥ | 22.40 | 27.60 | 24.50 | 25.50 |
| Cmax:Cmin | 1.82 | 2.75 | 1.73 | 2.73 |
| Fluctuation | 0.78 | 1.18 | 0.75 | 1.198 |
| Index (FI) | | | | |

The data from this study (Table 22/FIG. 26) and Table 23 demonstrates that Tablet 37 exhibits a lower FI, within a 24 hour dosing period, under MF-MC conditions and under HF-HC conditions, compared to the marketed pyridostigmine product, e.g., MESTINON® (60 mg×3).

### Example 20: Alcohol-Induced Dose Dumping of Pyridostigmine from the Gastroretentive Compositions of the Disclosure

The present example provides evaluation of alcohol-induced dose dumping of Tablets 38 and 39 comprising pyridostigmine bromide.

TABLE 24
Formulation of Pyridostigmine Bromide Tablet

| | Tablet 38 | Tablet 39 |
|---|---|---|
| Ingredients | (mg/dose) | (mg/dose) |
| Tablet Core | | |
| Pyridostigmine bromide | 94.50 | 306.0 |
| Succinic acid | 80.0 | 80.0 |
| Sodium bicarbonate | 55.0 | 55.0 |
| Calcium carbonate | 65.0 | 65.0 |
| Crospovidone | 100.0 | 100.0 |
| Mannitol | 310.7 | 72.70 |
| BENECEL ™ K4M PH DC | 150.0 | 150.0 |
| METHOCEL ™ K100 Prem | 150.0 | 150.0 |
| LVCR | | |
| CAB-O-SIL ® | 15.0 | 15.0 |
| Talc | 10.0 | 10.0 |
| Magnesium stearate | 8.00 | 8.0 |
| Total Core Weight | 1050.0 | 1050.0 |
| Functional Coat | | |
| EUDRAGIT ® RL PO | 148.15 | 148.15 |
| Triethyl citrate | 22.22 | 22.22 |
| Talc | 29.63 | 29.63 |
| Total Functional Coat | 200 | 200 |
| Weight | | |
| Seal Coat | | |
| OPADRY ® white | 2.0 | 2.0 |
| Drug Layer | | |
| Pyridostigmine bromide | 10.5 | 34.0 |
| Hydroxypropyl cellulose | 2.1 | 6.8 |
| Total Drug Layer Weight | 12.6 | 40.8 |
| Over Coat | | |
| OPADRY ® Clear | 20.4 | 22.2 |
| Cosmetic Coat | | |
| OPADRY ® Beige | 20.0 | |
| OPADRY ® Yellow | | 20.0 |
| Total Tablet Weight | 1305.0 | 1335.0 |

Tablets 38 and 39 were made as per Tablet 23 in Example 12. Tablets 38 and 39 were tested for dissolution in various dissolution mediums, e.g., 0.1N HCl; 0.1N HCl containing 5% dissolved alcohol, 20% dissolved alcohol, and 40% dissolved alcohol; 50 mM pH 5 buffer; and 50 mM pH 5 buffer containing 5% dissolved alcohol, 20% dissolved alcohol, and 40% dissolved alcohol. The tablets were tested for dissolution using USP Apparatus I, Custom basket, at 100 rpm and 37° C., in 900 ml of the dissolution medium. Tables 25-28 summarize the data obtained from the dissolution studies in various mediums.

TABLE 25
Dissolution of Tablet 38

| | | % Dissolution | % Dissolution | % Dissolution |
|---|---|---|---|---|
| Time | % Dissolution | 0.1N HCl + | 0.1N HCl + | 0.1N HCl + |
| (Hrs) | 0.1N HCl | 5% EtOH | 20% EtOH | 40% EtOH |
| 0 | 0 | 0 | 0 | 0 |
| 0.25 | 9 | 9 | 9 | 9 |
| 0.5 | 9 | 9 | 10 | 11 |
| 0.75 | 10 | 11 | 14 | 18 |
| 1.0 | 11 | 13 | 20 | 25 |
| 1.25 | 13 | 15 | 27 | 30 |
| 1.5 | 15 | 18 | 32 | 34 |
| 2 | 19 | 23 | 40 | 39 |

TABLE 26
Dissolution of Tablet 38

| | | % Dissolution | % Dissolution | % Dissolution |
|---|---|---|---|---|
| | % Dissolution | 50 mM pH 5 | 50 mM pH 5 | 50 mM pH 5 |
| Time | 50 mM pH 5 | Buffer + 5% | Buffer + 20% | Buffer + 40% |
| (Hrs) | Buffer | EtOH | EtOH | EtOH |
| 0 | 0 | 0 | 0 | 0 |
| 0.25 | 9 | 10 | 10 | 11 |
| 0.5 | 13 | 13 | 16 | 15 |
| 0.75 | 15 | 15 | 22 | 23 |
| 1.0 | 16 | 16 | 27 | 29 |
| 1.25 | 18 | 17 | 31 | 33 |
| 1.5 | 20 | 18 | 34 | 36 |
| 2 | 24 | 20 | 39 | 42 |

TABLE 27
Dissolution of Tablet 39

| | | % Dissolution | % Dissolution | % Dissolution |
|---|---|---|---|---|
| Time | % Dissolution | 0.1N HCl + | 0.1N HCl + | 0.1N HCl + |
| (Hrs) | 0.1N HCl | 5% EtOH | 20% EtOH | 40% EtOH |
| 0 | 0 | 0 | 0 | 0 |
| 0.25 | 10 | 9 | 10 | 10 |
| 0.5 | 10 | 9 | 11 | 13 |
| 0.75 | 9 | 9 | 16 | 22 |
| 1.0 | 9 | 10 | 23 | 28 |
| 1.25 | 10 | 11 | 28 | 34 |
| 1.5 | 11 | 14 | 33 | 38 |
| 2 | 13 | 20 | 41 | 45 |

TABLE 28
Dissolution of Tablet 39

| | | % Dissolution | % Dissolution | % Dissolution |
|---|---|---|---|---|
| | % Dissolution | 50 mM pH 5 | 50 mM pH 5 | 50 mM pH 5 |
| Time | 50 mM pH 5 | Buffer + 5% | Buffer + 20% | Buffer + 40% |
| (Hrs) | Buffer | EtOH | EtOH | EtOH |
| 0 | 0 | 0 | 0 | 0 |
| 0.25 | 9 | 11 | 10 | 9 |
| 0.5 | 11 | 12 | 17 | 16 |
| 0.75 | 13 | 15 | 23 | 24 |
| 1.0 | 17 | 17 | 27 | 30 |
| 1.25 | 20 | 19 | 30 | 34 |
| 1.5 | 24 | 21 | 33 | 38 |
| 2 | 31 | 25 | 38 | 43 |

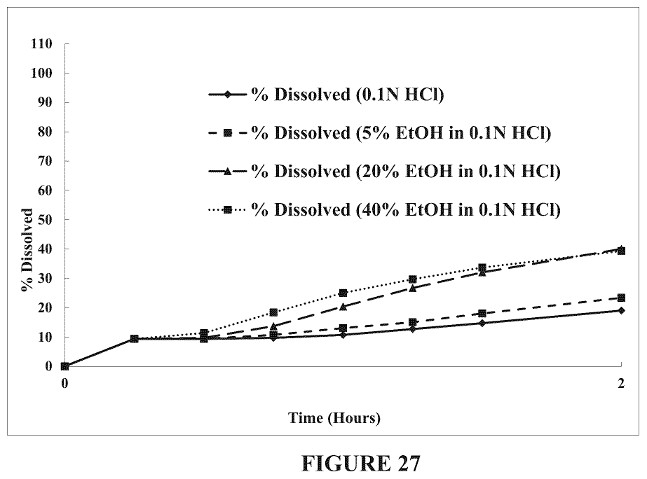

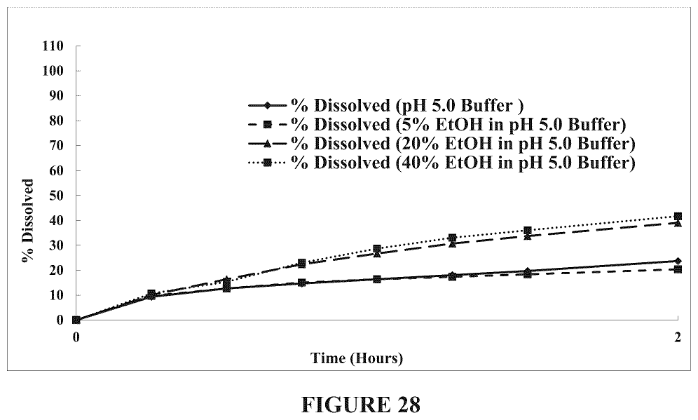

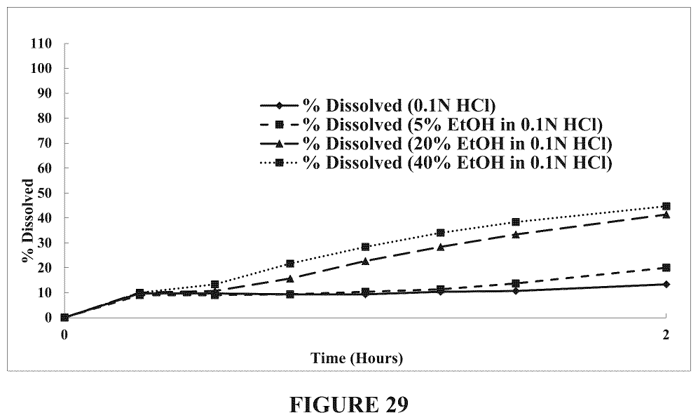

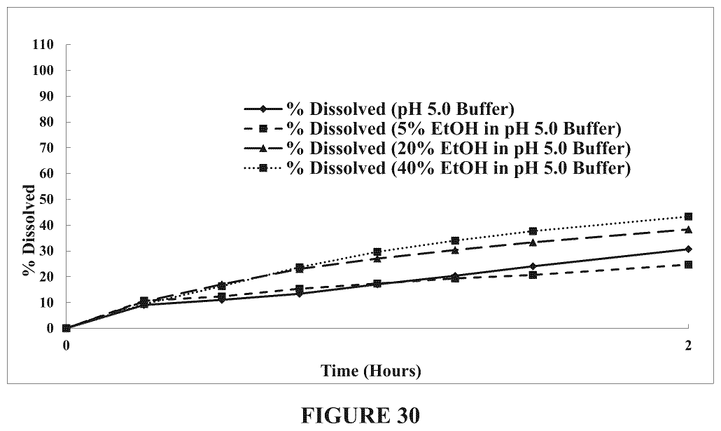

The data from Tables 25-28/FIGS. 27-30 clearly demonstrates that the gastroretentive tablet compositions of the disclosure, e.g., Tablets 38 and 39, provide resistance against alcohol dose dumping.

### Example 21 Steady State Simulation Over a 24 Hour Period from Day 5 to Day 6, Under Medium Fat-Medium Calorie and High Fat-High Calorie Conditions

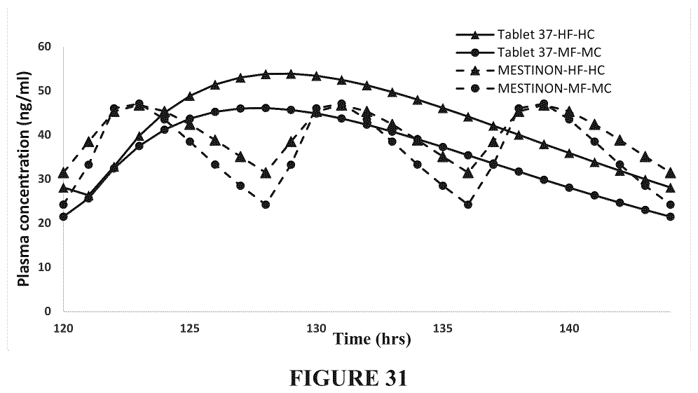

A single dose and multiple dose cross-over pharmacokinetic (PK) study was conducted in healthy volunteers under fed conditions (MF-MC and HF-HC conditions) to evaluate and compare PK performance of extended release compositions of the disclosure using Tablet 37, administered once-a-day, and marketed pyridostigmine bromide tablet, MESTINON® (60 mg), administered thrice-a-day. An open-label, randomized, two treatment, four period, four-arm, single dose and multiple dose, cross-over, bioavailability study was conducted in 16 normal, healthy, adult, human subjects under medium-fat-medium calorie (600-650 Kcal; ˜40% fat) and high-fat high-calorie (800-900 Kcal; ˜50% fat) breakfast conditions. FIG. 31 provides a steady state plasma concentration, under fed conditions (MF-MC and HF-HC conditions), of pyridostigmine bromide from Tablet 37 and MESTINON (60 mg×3), based on a steady state simulation over a 24 hour period from day 5 to day 6.

Pharmacokinetic parameters for pyridostigmine are summarized in Table 29.

TABLE 29
Pharmacokinetics Results (N = 16 subjects)

| | MF-MC | | HF-HC | |
|---|---|---|---|---|
| | 600-650 Kcal; ~40% fat | | 800-900 Kcal; ~50% fat | |
| | Tablet 37 | MESTINON ® | Tablet 37 | MESTINON ® |
| PK Parameters | (180 mg) | (60 mg × 3) | (180 mg) | (60 mg × 3) |
| Cₘₐₓ (ng/mL) | 46.0 | 47.0 | 54.0 | 47.0 |
| Cₘᵢₙ (ng/mL) | 21.0 | 24.0 | 26.0 | 31.0 |
| (% CV) | | | | |
| Cₐᵥ | 35.5 | 36.3 | 42.3 | 40.1 |

### Example 22: Gastroretentive Pyridostigmine Compositions with IR Drug Layer

The present Example provides gastroretentive pyridostigmine compositions comprising an extended release core and an immediate release drug layer. The extended release core comprises drug intermediate blend comprising pyridostigmine or a pharmaceutically acceptable salt thereof, Cab-O-Sil, and talc. Four different compositions were prepared as outlined in Table 30.

TABLE 30
Formulation of Pyridostigmine Bromide Tablets

| | | | | |
|---|---|---|---|---|
| | Tablet 38 | Tablet 39 | Tablet 40 | Tablet 41 |
| Ingredients | 105 mg | 205 mg | 275 mg | 340 mg |

Tablet Core
Drug Intermediate Blend

| Pyridostigmine bromide | 94.5 | 184.5 | 247.5 | 306 |
|---|---|---|---|---|
| CAB-O-SIL ® | 7.09 | 13.84 | 18.56 | 22.95 |
| Talc | 4.73 | 9.23 | 12.38 | 15.30 |
| Excipients for Final Blend | | | | |
| Succinic acid | 80.0 | 80.0 | 80.0 | 80.0 |
| Sodium bicarbonate | 55.0 | 55.0 | 55.0 | 55.0 |
| Calcium carbonate | 65.0 | 65.0 | 65.0 | 65.0 |
| Crospovidone | 100.0 | 100.0 | 100.0 | 100.0 |
| MANNOGEM ®XL | 310.7 | 209.4 | 138.6 | 72.7 |
| BENECEL ™ K4M PH DC | 150.0 | 150.0 | 150.0 | 150.0 |
| METHOCEL ™ K100 Prem | 150.0 | 150.0 | 150.0 | 150.0 |
| LVCR | | | | |
| CAB-O-SIL ® | 15.00 | 15.00 | 15.0 | 15.0 |
| Talc | 10.0 | 10.0 | 10.0 | 10.0 |
| Magnesium stearate | 8.00 | 8.00 | 8.0 | 8.0 |
| Total Weight | 1050.0 | 1050.0 | 1050.0 | 1050.0 |
| Functional Coat | | | | |
| EUDRAGIT ® RL PO | 148.15 | 148.15 | 148.15 | 148.15 |
| Triethyl citrate | 22.22 | 22.22 | 22.22 | 22.22 |
| Talc | 29.63 | 29.63 | 29.63 | 29.63 |
| Solvent* acetone:water (95:5) | — | — | — | — |
| Seal Coat-1 | | | | |
| OPADRY ® White | 2.0 | 2.0 | 2.0 | 2.0 |
| Solvent*Isopropyl | — | — | — | — |
| alcohol:purified water (50:50) | | | | |
| Drug Layer | | | | |
| Pyridostigmine bromide | 10.5 | 20.5 | 27.5 | 34.0 |
| Hydroxypropyl cellulose | 2.1 | 4.1 | 95.5 | 6.8 |
| Solvent*Acetone:Dehydrated | — | — | — | — |
| alcohol (80:20) | | | | |
| Seal Coat-2 | | | | |
| OPADRY ® Clear | 20.4 | 18.4 | 20.0 | 22.2 |
| Solvent*Acetone | — | — | — | — |
| Cosmetic Coat | — | — | — | — |
| OPADRY ® Beige | 20.0 | — | — | — |
| OPADRY ® Blue | — | 20.0 | — | — |
| OPADRY ® Pink | — | — | 20.0 | — |
| OPADRY ® Yellow | — | — | — | 20.0 |
| Solvent*Isopropyl alcohol, | — | — | — | — |
| and Water (70:30) | | | | |
| Total Weight | 1305.0 | 1315.0 | 1325.0 | 1335.0 |

*Removed during process

Tablets 38-41 were made according to the following general procedure.

### Manufacturing Procedure:

### A. Tablet Cores

1. Pyridostigmine bromide, talc and Cabo-O-Sil were blended into drug intermediate blend using a high shear mixer.
2. BENECEL™ K4M-DC and METHOCEL™ K100 Premium LVCR sieved through a suitably sized mesh screen, and drug intermediate blend form step #1 were blended using a V-blender to obtain a uniform blend.
3. Succinic acid (micronized), sodium bicarbonate, and calcium carbonate were sieved through a suitably sized mesh screen, added to the V-blender containing the blend from step #2, and further blended to obtain a uniform blend.
4. Crospovidone, MANNOGEM® XL, talc, and CAB-O-SIL® were sieved through a suitably sized mesh screen, added to the V-blender containing the blend from step #3, and further blended to obtain a uniform blend.
5. Magnesium stearate was sieved through a suitably sized mesh screen, added to the V-blender containing the blend from step #4, and further blended to obtain a final blend.
6. The resulting blend from step #5 was compressed using a suitable tablet press to obtain pyridostigmine tablet cores.

### B. Functional Coating

1. EUDRAGIT® RL PO was taken in acetone and water mixture (95:5) and mixed in a stainless-steel container equipped with air-mixer to obtain a clear solution.
2. To the solution from step #1, triethyl citrate was added and mixed for at least 45 minutes.
3. To the solution from step #2, talc was added and mixed for at least 30 minutes to obtain a homogeneous dispersion.
4. The pyridostigmine tablet cores from Step A were taken in a perforated coating pan (O'Hara Coater) and sprayed with the homogeneous dispersion from step #3 to obtain functional coated tablets.
5. The functional coated tablets from step #4 were dried in the perforated coating pan.

### C. Seal Coat-1

1. A solvent mixture of Isopropyl alcohol and purified water (50:50) was prepared in a stainless steel container equipped with air mixer.
2. OPADRY® White was gradually added to the solvent mixture from step #1 and mixed for not less than 60 minutes to obtain a uniform dispersion.
3. Functional coated tablet cores from Step B were seal coated, using a perforated coating (O'Hara Coater), with the dispersion from step #2 to obtain seal coated tablet cores.

### D. IR Drug Layer

1. A solvent mixture of acetone and dehydrated alcohol (80:20) was prepared in a stainless steel container equipped with air mixer.
2. Hydroxypropyl cellulose was added to the solvent mixture from step #1 and mixed for not less than 60 minutes to obtain a coating solution.
3. Seal coated pyridostigmine tablet cores from Step C were further coated, using a perforated coating pan (O'Hara Coater), with the coating solution from step #2 to obtain IR coated tablet cores.

### E. Seal Coat-2

1. Acetone was taken in a stainless steel container equipped with air mixer.
2. OPADRY® Clear was gradually added to the solvent from step #1 and mixed for not less than 60 minutes to obtain a uniform dispersion.
3. IR coated tablet cores from Step D were seal coated, using a perforated coating pan (O'Hara Coater), with the dispersion from step #2 to obtain seal coated tablet cores.

### F. Over Coat/Cosmetic Coat

1. A solvent mixture of Isopropyl alcohol and purified water (70:30) was prepared in a stainless steel container equipped with air mixer.
2. Opadry® Beige, or Opadry® Blue, Opadry® Pink, and Opadry® Yellow were added to the solvent mixture from step #1 for pyridostigmine tablets, 105 mg, 205 mg, 275 mg and 340 mg, respectively, and mixed for not less than 60 minutes.
3. Tablet cores with Seal Coat-2 were further coated, using a perforated coating pan (O'Hara Coater), with the coating solution from step #2, and dried in the coating pan to a moisture content of below 1.5%.

### Example 23: Gastroretentive Pyridostigmine Compositions with IR Drug Layer

The present Example provides gastroretentive pyridostigmine compositions comprising an extended release core and an immediate release drug layer. The extended release core comprises drug intermediate blend comprising pyridostigmine or a pharmaceutically acceptable salt thereof, and Cab-O-Sil. Four different compositions were prepared as outlined in Table 31.

TABLE 31
Formulation of Pyridostigmine Bromide Tablets

| | | | | |
|---|---|---|---|---|
| | Tablet 42 | Tablet 43 | Tablet 44 | Tablet 45 |
| Ingredients | 105 mg | 205 mg | 275 mg | 340 mg |

Tablet Core
Drug Intermediate Blend

| Pyridostigmine bromide | 95.445 | 186.345 | 249.975 | 309.06 |
|---|---|---|---|---|
| CAB-O-SIL ® | 9.555 | 18.655 | 25.025 | 30.94 |
| Excipients for Final Blend | | | | |
| Succinic acid | 80.0 | 80.0 | 80.0 | 80.0 |
| Sodium bicarbonate | 55.0 | 55.0 | 55.0 | 55.0 |
| Calcium carbonate | 65.0 | 65.0 | 65.0 | 65.0 |
| Crospovidone | 100.0 | 100.0 | 100.0 | 100.0 |
| MANNOGEM ® XL | 293 | 194 | 124.7 | 60.4 |
| BENECEL ™ K4M PH DC | 150.0 | 150.0 | 150.0 | 150.0 |
| METHOCEL ™ K100 Prem | 150.0 | 150.0 | 150.0 | 150.0 |
| LVCR | | | | |
| CAB-O-SIL ® | 5 | 5 | 5 | 5 |
| Talc | 40 | 40 | 40 | 40 |
| Magnesium stearate | 8.00 | 8.00 | 8.0 | 8.0 |
| Total Weight | 1050.0 | 1050.0 | 1050.0 | 1050.0 |
| Functional Coat | | | | |
| EUDRAGIT ® RL PO | 148.15 | 148.15 | 148.15 | 148.15 |
| Triethyl citrate | 22.22 | 22.22 | 22.22 | 22.22 |
| Talc | 29.63 | 29.63 | 29.63 | 29.63 |
| Solvent* acetone:water (95:5) | — | — | — | — |
| Seal Coat-1 | | | | |
| OPADRY ® White | 2.0 | 2.0 | 2.0 | 2.0 |
| Solvent*Isopropyl | — | — | — | — |
| alcohol:Purified water | | | | |
| (50:50) | | | | |
| Drug Layer | | | | |
| Pyridostigmine bromide | 10.5 | 20.5 | 27.5 | 34.0 |
| Hydroxypropyl cellulose | 2.1 | 4.1 | 95.5 | 6.8 |
| Solvent*Acetone:Dehydrated | — | — | — | — |
| alcohol (80:20) | | | | |
| Seal Coat-2 | | | | |
| OPADRY ® Clear | 20.4 | 18.4 | 20.0 | 22.2 |
| Solvent*Acetone | — | — | — | — |
| Cosmetic Coat/Over Coat | — | — | — | — |
| OPADRY ® Beige | 20.0 | — | — | — |
| OPADRY ® Blue | — | 20.0 | — | — |
| OPADRY ® Pink | — | — | 20.0 | — |
| OPADRY ® Yellow | — | — | — | 20.0 |
| Solvent*Isopropyl alcohol, | — | — | — | — |
| and Water (70:30) | | | | |
| Total Weight | 1305.0 | 1315.0 | 1325.0 | 1335.0 |

*Removed during process

Tablets 38-41 were made according to the following general procedure.

### Manufacturing Procedure:

### A. Tablet Cores

1. Pyridostigmine bromide, and Cabo-O-Sil were blended into drug intermediate blend using a high shear mixer.
2. BENECEL™ K4M-DC and METHOCEL™ K100 Premium LVCR, sieved through a suitably sized mesh screen, and drug intermediate blend from step #1 were added to a V-blender and blended to obtain a uniform blend.
3. Succinic acid (micronized), sodium bicarbonate, and calcium carbonate were sieved through a suitably sized mesh screen, added to the V-blender containing the blend from step #2, and further blended to obtain a uniform blend.
4. Crospovidone, MANNOGEM® XL, talc, and CAB-O-SIL® were sieved through a suitably sized mesh screen, added to the V-blender containing the blend from step #3, and further blended to obtain a uniform blend.
5. Magnesium stearate was sieved through a suitably sized mesh screen, added to the V-blender containing the blend from step #4, and further blended to obtain a final blend.
6. The final blend from step #5 was compressed using a suitable tablet press to obtain pyridostigmine tablet cores.

### B. Functional Coating

1. EUDRAGIT® RL PO was taken in acetone and water mixture (95:5) and mixed in a stainless-steel container equipped with air-mixer to obtain a clear solution.
2. To the solution from step #1, triethyl citrate was added and mixed for at least 45 minutes.
3. To the solution from step #2, talc was added and mixed for at least 30 minutes to obtain a homogeneous dispersion.
4. The pyridostigmine tablet cores from Step A were taken in a perforated coating pan (O'Hara Coater) and sprayed with the homogeneous dispersion from step #3 to obtain functional coated tablets.
5. The functional coated tablets from step #4 were dried in the perforated coating pan.

### C. Seal Coat-1

1. A solvent mixture of Isopropyl alcohol and purified water (50:50) was prepared in a stainless steel container equipped with air mixer.
2. OPADRY® White was gradually added to the solvent mixture from step #1 and mixed for not less than 60 minutes to obtain a uniform dispersion.
3. Functional coated tablet cores from Step B were seal coated, using a perforated coating pan (O'Hara Coater), with the dispersion from step #2 to obtain seal coated tablet core.

### D. Tray Drying

Tablets with Seal Coat-1 from Step C were dried on trays lined with polyethylene bags for not less than 10 hours.

### E. Laser Drill

Dried tablets from Step D were laser drilled on one side of the tablet to provide an with orifice size of from about 0.3 mm to about 0.6 mm.

### F. IR Drug Layer

1. A solvent mixture of acetone and dehydrated alcohol (80:20) was prepared in a stainless steel container equipped with air mixer.
2. Hydroxypropyl cellulose was added to the solvent mixture from step #1 and mixed for not less than 60 minutes to obtain a coating solution.
3. Laser drilled pyridostigmine tablet cores from Step E were further coated, using a perforated coating pan (O'Hara Coater), with the coating solution from step #2 to obtain IR coated tablet cores.

### G. Seal Coat-2

1. Acetone was taken in a stainless steel container equipped with air mixer.
2. OPADRY® Clear was gradually added to the solvent from step #1 and mixed for not less than 60 minutes to obtain a uniform dispersion.
3. IR coated tablet cores from Step F were seal coated, using a perforated coating (O'Hara Coater), with the dispersion from step #2 to obtain seal coated tablet cores.

### H. Over Coat/Cosmetic Coat

1. A solvent mixture of Isopropyl alcohol and purified water (70:30) was prepared in a stainless steel container equipped with air mixer.
2. Opadry® Beige, or Opadry® Blue, Opadry® Pink, and Opadry® Yellow were added to the solvent mixture from step #1 for pyridostigmine tablets, 105 mg, 205 mg, 275 mg and 340 mg, respectively, and mixed for not less than 60 minutes.
3. Tablet cores with Seal Coat-2 were further coated, using a perforated coating pan (O'Hara Coater), with the coating solution from step #2, and dried in the coating pan to a moisture content of below 1.5%.

### Example 24: Single Ascending Dose (SAD) Study of Pyridostigmine Under Fed Conditions

An open-label, non-randomized, four dose levels, four period, single ascending dose (SAD) study was conducted in 14 healthy, adult human subjects under fed (HFHC) conditions, to evaluate dose proportionality of the proposed pyridostigmine bromide tablets, 105 mg, 205 mg, 275 mg and 340 mg.

The primary objective of this study was to assess dose proportionality of the proposed pyridostigmine bromide tablets, 105 mg, 205 mg, 275 mg and 340 mg, in healthy, adult, human subjects under fed conditions.

The secondary objective of this study was to monitor the safety and tolerability of single of the proposed pyridostigmine bromide tablets, 105 mg, 205 mg, 275 mg and 340 mg in healthy, adult, human subjects under fed conditions.

### Methodology

An open-label, non-randomized, four dose levels, four period, single ascending dose study to evaluate the dose proportionality of the proposed pyridostigmine bromide tablets, 105 mg, 205 mg, 275 mg and 340 mg in healthy, adult, human subjects under high fat high calorie (HFHC) in fed conditions [high fat and caloric content was defined as each meal containing (800-900 Kcal with ˜fat 50%)]. As per protocol, 14 subjects (EID 001-EID 014) were enrolled in the study after obtaining written informed consent in the language best understood by the volunteer.

11 Subjects complete all the treatment period of the study. One subject was dropped out prior to dosing in period 3. One subject was withdrawn due to due to serious adverse event (Drug Reaction Fasciculation) after dosing in period 4. One subject was withdrawn due to AE (Fasciculation) after dosing in period 4. The outcome of all drug related adverse events (AEs) and serious adverse events (SAE) was moderate in nature and the relationship to the treatment product was certain. The outcome of all drug related AEs and SAE was recovered/resolved. Based on evaluation of adverse events, clinical laboratory evaluation and vital signs examination, it was concluded that all the Test products (Tablets 38-41) were well tolerated and were found to be safe.

A washout period of at least 05 days was maintained in between the IP administrations of each treatment periods for both Group 1 and Group 2 subjects. Subjects received either Tablet 38 [1× Tablet 38 (105 mg)] or Tablet 39 [1× Tablet 39 (205 mg)] or Tablet 40) [1× Tablet 40 (275 mg)] or Tablet 41 [1× Tablet 41 (340 mg)], under yellow monochromatic light in each study period.

The dose proportionality of pyridostigmine bromide Tablets-105 mg, 205 mg, 275 mg and 340 mg were calculated using primary PK parameters Cₘₐₓ, AUC₀₋ₜ and AUC_{0-∞} by analysis of variance and 90% confidence intervals.

Pharmacokinetic parameters of pyridostigmine from Tablet 38 (105 mg), Tablet 39 (205 mg), Tablet 40 (275 mg), and Tablet 41 (340 mg) are summarized in Table 32.

TABLE 32
Mean ± SD

| | Treatment | | | |
|---|---|---|---|---|
| | Tablet 38 | Tablet 39 | Tablet 40 | Tablet 41 |
| Parameters | (105 mg) | (205 mg) | (275 mg) | (340 mg) |
| Cₘₐₓ | 22.538 ± 4.857 | 42.864 ± 12.856 | 55.385 ± 16.037 | 71.281 ± 21.713 |
| AUC₀₋ₜ (ng · hr/mL) | 449.414 ± 89.662 | 791.726 ± 212.099 | 1004.464 ± 329.660 | 1101.821 ± 364.812 |
| AUC_{0-inf} (ng · hr/mL) | 465.620 ± 94.254 | 818.645 ± 218.560 | 1040.107 ± 333.953 | 1138.644 ± 387.213 |
| Tₘₐₓ (hr) | 17.583 ± 3.188 | 14.285 ± 3.930 | 15.307 ± 3.660 | 13.000 ± 3.286 |
| Kₑₗ (hr⁻¹) | 0.151 ± 0.030 | 0.139 ± 0.024 | 0.134 ± 0.038 | 0.132 ± 0.022 |
| t_{1/2} (hr) | 4.752 ± 0.855 | 5.115 ± 0.924 | 5.652 ± 1.987 | 5.361 ± 0.959 |
| AUC Extrapolated (%) | 3.428 ± 1.408 | 3.349 ± 1.452 | 3.649 ± 2.543 | 3.040 ± 1.307 |

Note:
EID 002 failed to complete the Period 3 in the study. Hence, EID 002 was excluded from the Pharmacokinetic and statistical analysis. EID 002, 012 and 013 failed to complete the Period 4 in the study. Hence, subject's data in period 4 was not considered for the Pharmacokinetic and statistical analysis.

To assess dose proportionality for different strengths the primary PK parameters, e.g., Cₘₐₓ, AUC₀₋ₜ and AUC_{0-∞} were calculated and summarized. Graphs for these PK parameters (e.g., mean value of Cₘₐₓ, AUC₀₋ₜ and AUC_{0-∞}) versus dose were presented. In addition, a power model (Proc-Mixed) with log (PK parameter) as response variable and log (dose) as predictor was fitted to the data for primary PK parameters. The slope was estimated with its 2-sided 90% and 95% CI.

Table 34 provides a summary of dose proportionality assessment for pyridostigmine 90% Confidence Interval and 95% Confidence Interval of Slope.

TABLE 34

| | Estimate | Estimate | P-Value | 90% CI | 95% CI |
|---|---|---|---|---|---|
| PK | of | of | of | for | for |
| Parameter | Intercept | Slope | Slope | Slope | Slope |
| Cₘₐₓ | −1.1966 | 0.9216 | <0.0001 | (0.8591, | (0.8465, |
| | | | | 0.9841) | 0.9967) |
| AUC₀₋ₜ | 2.7122 | 0.7316 | <0.0001 | (0.6484, | (0.6316, |
| | | | | 0.8149) | 0.8317) |
| AUC_{0-∞} | 2.7575 | 0.7298 | <0.0001 | (0.6455, | (0.6285, |
| | | | | 0.8141) | 0.8310) |

Data from Table 37 shows that estimate of slope for Cₘₐₓ is almost near 1 (e.g., 0.9216), and 90% and 95% Confidence Interval for the slope include 1. Hence, dose proportionality is concluded for Cₘₐₓ.

### Example 25: Multiple Ascending Dose (MAD) Study of Pyridostigmine Under Fed Conditions

An open-label, non-randomized, four treatment, four cohort, single period, sequential multiple ascending study to characterize pharmacokinetics, safety, and tolerability of pyridostigmine after once-daily administration of pyridostigmine bromide Tablet 38 (105 mg), Tablet 39 (205 mg), Tablet 40 (275 mg) and Tablet 41 (340 mg), for six consecutive days in healthy, adult, human subjects under fed (HFHC) conditions.

The primary objective of this study was characterizing the pharmacokinetics of pyridostigmine upon once-daily administration of pyridostigmine Tablet 38 (105 mg), Tablet 39 (205 mg), Tablet 40 (275 mg) and Tablet 41 (340 mg), for six consecutive days in healthy, adult, human subjects under fed conditions.

The secondary objective of this study was to monitor the safety and tolerability of pyridostigmine upon once-daily administration of Tablet 38 (105 mg), Tablet 39 (205 mg), Tablet 40 (275 mg) and Tablet 41 (340 mg), for six consecutive days in healthy, adult, human subjects under fed conditions.

Twelve healthy volunteers were enrolled in Cohorts 1-3, and 2 subjects were enrolled in Cohort 4. Cohort 1 was given Pyridostigmine bromide Tablet 38, 105 mg; Cohort 2 was given Pyridostigmine bromide Tablet 39, 205 mg; Cohort 3 was given Pyridostigmine bromide Tablet 40, 275 mg; and Cohort 4 was given Pyridostigmine bromide Tablet 41, 340 mg.

### Methodology

An open label, non-randomized, four treatment, four cohort, single period, sequential multiple ascending dose study to characterize the pharmacokinetics, safety and tolerability of pyridostigmine after once-daily administration of pyridostigmine bromide Tablet 38 (105 mg), Tablet 39 (205 mg), Tablet 40 (275 mg) and Tablet 41 (340 mg), for six consecutive days in healthy, adult, human subjects under fed conditions [high fat and caloric content was defined as each meal containing (800-900 Kcal with ˜fat 50%)]. The study was conducted in sequentially order i.e., cohorts 1, 2, 3 and 4 were executed sequentially. Thirty-eight normal healthy, human adult subjects between 18-45 years of age (both ages inclusive) were enrolled in the study-12 subjects (EID 001 to EID 012) in cohort 1, 12 subjects in (EID 013 to EID 024) cohort 2, 12 subjects (EID 025 to EID 036) in cohort 3 and 02 subjects (EID 037 and EID 038) were enrolled in cohort 4.

The Subjects received 1× Tablet 38 (105 mg) in cohort 1; 1× Tablet 39 (205 mg) in cohort 2; 1× Tablet 40 (275 mg) in cohort 3; and 1× Tablet 41 (340 mg) in cohort 4, under yellow monochromatic light.

Initially two subjects were enrolled (EID 037 and 038) in Cohort 4, as per the protocol requirement. However, both the subjects were withdrawn from the study due to multiple adverse events. (fasciculation, loose stool, muscle pain, abdomen pain) after the first dose on Day 1, related to the study drug. According to the study PI, adverse event numbers and severity could increase with consecutive days of dosing. Considering that was only first dose, serious adverse reaction or severe non-serious adverse reaction could be anticipated with consecutive days dosing. In light of patient safety, it was decided to not proceed with remaining enrolment and dosing of Cohort 4 subjects.

Vital signs [blood pressure, pulse rate, respiratory rate and temperature measurement (by infrared thermometer)] and general wellbeing was assessed before check-in, prior to dosing on Day 01 to Day 06 and during check-out of study. From Day 01 to Day 05, vital signs [blood pressure, pulse rate and temperature measurement (by infrared thermometer)] and general wellbeing was assessed after each dosing at 01.00, 02.00, 03.00, 04.00, 05.00, 06.00 and 13.00 hours after dosing. On Day 06, vital signs [blood pressure, pulse rate and temperature measurement (by infrared thermometer)] and general wellbeing was assessed at 01.00, 02.00, 03.00, 04.00, 05.00, 06.00, 13.00 and 25.00 hours after dosing.

TABLE 35
Pharmacokinetic Parameters of Pyridostigmine in Each Arm on Day 1
Mean ± SD

| | Treatment | | |
|---|---|---|---|
| | Tablet 38 | Tablet 39 | Tablet 40 |
| | (105 mg) | (205 mg) | (275 mg) |
| Parameters | (N = 12) | (N = 11)* | (N = 12) |
| Cₘₐₓ (ng/mL) | 29.617 ± 6.775 | 46.721 ± 7.827 | 72.198 ± 21.108 |
| AUC₀₋ₜ (ng · hr/mL) | 440.783 ± 120.528 | 669.972 ± 113.181 | 984.609 ± 247.835 |
| AUC_{0-inf} (ng · hr/mL) | 539.316 ± 234.958* | 878.425 ± 220.321 | 1706.261 ± 983.902\hat{ } |
| Tₘₐₓ (hr) | 16.666 ± 3.651 | 11.818 ± 0.603 | 12.585 ± 3.633 |
| Kₑₗ (hr⁻¹) | 0.109 ± 0.046* | 0.111 ± 0.039 | 0.073 ± 0.0386\hat{ } |
| t_{1/2} (hr) | 7.342 ± 2.906* | 7.582 ± 4.783 | 13.351 ± 10.335\hat{ } |
| AUC Extrapolated | 22.452 ± 13.022* | 21.225 ± 13.538 | 34.692 ± 20.234\hat{ } |
| (%) | | | |

*N = 5 There were no log-linear relationship in the terminal elimination phase to estimate secondary pharmacokinetic parameter for EID 001, 002, 004, 005, 006, 009 and 011.
#N = 11, EID 015 was withdrawn from the study due to adverse event (COVID 19 disease).
\hat{ }There were no log-linear relationship in the terminal elimination phase to estimate secondary pharmacokinetic parameter for EID 027 and 032.

TABLE 36
Pharmacokinetic Parameters of Pyridostigmine in Each Arm-Multiple Dose
Mean ± SD

| | Treatment | | |
|---|---|---|---|
| | Tablet 38 | Tablet 39 | Tablet 40 |
| | (105 mg) | (205 mg) | (275 mg) |
| Parameters | (N = 12) | (N = 11)* | (N = 12) |
| C_{max ss} (ng/mL) | 37.557 ± 10.247 | 66.167 ± 11.579 | 94.660 ± 17.540 |
| C_{τ} (ng/mL) | 18.371 ± 6.554 | 17.150 ± 5.493 | 36.921 ± 20.851 |
| Cₐᵥ (ng/mL) | 24.539 ± 6.453 | 37.402 ± 6.639 | 58.868 ± 16.085 |
| T_{max ss} (hr) | 132.666 ± 1.775 | 132.000 ± 0.000 | 132.378 ± 3.983 |
| C_{min ss} (ng/mL) | 12.622 ± 5.462 | 15.480 ± 2.941 | 28.366 ± 12.386 |
| AUC_{0-τ ss} (hr*ng/mL) | 588.958 ± 154.879 | 897.654 ± 159.341 | 1412.850 ± 386.054 |
| swing | 2.798 ± 2.660 | 3.340 ± 0.701 | 2.881 ± 1.518 |
| Fluctuation (%) | 104.831 ± 44.067 | 135.985 ± 16.348 | 120.280 ± 40.857 |

Steady state was achieved for Pyridostigmine Bromide Tablets 105 mg, 205 mg, and 275 mg (Tablets 39-41) for all the subjects except for one subject EID #027 (as on day 5 to day 6 trough ratio was not within the interval 80 to 120) in Pyridostigmine Bromide Tablets 275 mg strength. No significant accumulation was observed on Day 6 (accumulation ratio range 0.6938-1.7166) except for subject EID #030 (275 mg).

### Safety Results:

Subjects were monitored for adverse events throughout the study. At the end of the study, post study safety evaluation was done which included haematology and clinical biochemistry.

TABLE 37
List of Adverse Events

| | | | | | Relationship |
|---|---|---|---|---|---|
| | | | Adverse | Severity/ | of AE with |
| EID | Cohort | Period | event | Rating | drug |
| 001 | 1 | PSA | High ALT | Mild | Possible |
| 006 | 1 | PSA | High | Mild | Possible |
| | | | Serum | | |
| | | | Potassium | | |
| 015 | 2 | 1 | COVID 19 | Mild | Unlikely |
| | | | Disease | | |
| 014 | 2 | PSA | High ALT | Mild | Possible |
| 032 | 3 | 1 | Pain | Mild | Possible |
| | | | Abdomen | | |
| 035 | 3 | PSA | High | Mild | Unlikely |
| | | | Blood | | |
| | | | Glucose | | |
| 037 | 4 | 1 | Fasciculation | Mild | Certain |
| 037 | 4 | 1 | Abdominal | Mild | Certain |
| | | | Pain | | |
| 037 | 4 | 1 | Loose Stool | Mild | Certain |
| 037 | 4 | PSA | High ALT | Mild | Possible |
| 038 | 4 | 1 | Fasciculation | Mild | Certain |
| 038 | 4 | 1 | Muscle Pain | Mild | Certain |

PSA: Post-study analysis. Subject EID#015 (Cohort-2) was withdrawn from the study. Cohort-4 subjects were withdrawn from the study and dosing was not continued.

The outcome of all treatment related AEs was recovered/resolved. There were no deaths, other serious adverse events or other clinically relevant adverse events reported in the study. Based on evaluation of adverse events, clinical laboratory evaluation and vital signs examination, it was concluded that pyridostigmine bromide SR/ER Tablets 105 mg, pyridostigmine bromide SR/ER tablet 205 mg, and pyridostigmine bromide SR/ER Tablets 275 mg were well tolerated and found to be safe.

### Example 26: Nonclinical Study to Determine Maximum Tolerated Dose

Nonclinical study was conducted with 48 Beagle dogs (24 males+24 females), distributed into 4 main groups and 4 recovery groups [GRS Test Control (G1), Reference (G2), GRS Test-low and high dose groups (G3 and G4), GRS Control recovery (G1R), Reference recovery (G2R), GRS Test-low and high dose recovery groups (G3R and G4R)]. Each main group was comprised of 4 male and 4 female dogs and each recovery group was comprised of 2 male and 2 female dogs. The GRS Test includes proposed compositions of the disclosure, and GRS reference includes Reference product MESTINON® (60 mg).

### Treatment

The respective compositions were administered orally to G1 and G1R (Dose: 0 mg/dog/day—one pyridostigmine control tablet once a day), G2 and G2R (Dose: 120 mg/dog/day or 60 mg/dog—one tablet BID), G3 and G3R (Dose: 105 mg/dog/day—one tablet QD) and G4 and G4R (Dose: 275 mg/dog/day—one tablet QD) group animals, respectively for minimum of 90 days. The animals were in fed condition at each administration of doses and the feed was offered approximately 30 minutes prior at each administration. The 10 mL/kg body weight of RO water (Water purified by reverse osmosis technique) was administered immediately after each tablet administration to facilitate size expansion.

### Observations

The key assessments included clinical signs, changes in body weight and food consumption, feces observations for the presence of tablets, changes in the electrocardiogram parameters, clinical/physical/ophthalmological examination, hematology, coagulation, clinical chemistry, urinalysis, toxicokinetics, gross pathology, organ weights and histopathology. All main group dogs were euthanized a day after their final dose and recovery group dogs were euthanized after completion of the recovery period. Blood was collected for plasma concentration analyses of MESTINON®/Reference product or pyridostigmine compositions of the disclosure/Test product, at the following time points: predose, 0.5, 1, 2, 4, 8, 12, 14, 16, 20 and 24 h post dose on Day 1, 45 and Day 90, while at pre-dose and 8 h post dose (2 points) samples were collected in Test product control animals (G1).

### Results

No mortalities and/or abnormalities were detected during physical and ophthalmological examination. In the MESTINON® group (120 mg/dog/day), muscle fasciculation (slight to moderate) was observed in forelimb and hindlimb muscles in most of the dogs throughout the treatment period. Few incidences of emesis (slight to severe) with froth (yellow)/undigested food and poorly formed feces containing water and all water, very little solid content (red and mucus). Also, few incidences of salivation, slight was observed.

In Test group (105 mg/dog/day-G3), muscle fasciculation (slight to moderate) was observed in forelimbs and hindlimb muscles. Occasionally, emesis (slight) with partially digested tablet/intact tablet and poorly formed feces containing water was observed in few dogs. Also, single incidence of slight salivation was observed during the treatment period.

In Test group (275 mg/dog/day-G4), muscle fasciculation of slight to severe intensity was observed in forelimbs and hindlimb muscles in most of the dogs throughout the treatment period. Few incidences of emesis (slight to moderate) with froth (yellow)/undigested food/partially digested tablets/intact tablets and poorly formed feces containing water and weakness was observed. Two incidences of salivation were observed.

Test control tablets were observed in the feces of dogs dosed throughout the treatment period and most of the tablets were partial. Test tablet remains were found in the feces at the dose levels of 105 mg/dog/day during the treatment period and most of the tablets were partial in nature. In comparison to 105 mg/dog/day, more tablets, e.g., intact tablets, were observed in feces at the Test group G4 at the dose of 275 mg/dog/day. Additionally, tablets in the feces were more in Test groups when compared to control group. No MESTINON® tablets/reference tablets were observed.

Decrease in body weight/body weight gains were observed in female dogs at Test dose of 275 mg/dog/day (G4 group). The changes in the body weight/body weight gain were almost comparable between MESTINON® group/reference group at the dose of 120 mg/dog/day and Test group at the dose of 105 mg/dog/day (G3 group),

No changes in the food consumption were observed in male and female dogs treated with MESTINON® at the dose of 120 mg/dog/day and Test tablets at the dose of 105 and 275 mg/dog/day during the treatment period.

### Test Tablets:

Plasma concentrations time profile were consistent with oral administration and were quantifiable until 24 h. The mean Tmax was observed around 12.0 to 18.0 h. Dose related increase in plasma exposure (AUClast) and peak plasma concentration (Cmax), was observed in less than dose proportional manner in both genders on day 1 and Day 45 and dose proportional on day 90. Gender related differences in pharmacokinetics were nonsignificant. Repeated administration of the Test product, for 45 days and 90 days, did not show a tendency of drug accumulation at tested dose levels. Pyridostigmine dose normalized exposure ratio following administration of Test and Reference (Test Product/Mestinon) were ranged from 1.54 to 2.90 in both genders across the tested dose levels on both study days, suggesting Pyridostigmine exposure following administration of Test tablet is about 1.5 to 2.9-fold higher compared to reference product Mestinon.

### Mestinon/Reference Tablets

Plasma concentration time profile was consistent with BID dosing and were quantifiable until 24 h post dose. The mean Tmax was observed around 2.0 to 16.0 h. Gender related differences were insignificant. Following repeated administration for 45 days and 90 days, it did not show a tendency of drug accumulation.

The toxicokinetic parameters are presented in the following table.

TABLE 38
Toxicokinetic Parameters

| | | | Dose | Tₘₐₓ | Cₘₐₓ | AUC_{0-12 h} | AUCₗₐₛₜ | Cₗₐₛₜ | Tₗₐₛₜ | T_{1/2} |
|---|---|---|---|---|---|---|---|---|---|---|
| Drug | Day | Gender | (mg/dog/day) | (hr) | (ng/mL) | (ng · h/mL) | (ng · h/mL) | (ng/mL) | (h) | (h) |
| Mestinon | 1 | Male | 120 | 14.0 ± | 191 ± | 1150 ± | 2780 ± | 90.8 ± | 24 ± | 8.19 ± |
| | | | | 5.75 | 20.5 | 233 | 310 | 14 | 0 | 1.94 |
| | | Female | 120 | 16.0 ± | 238 ± | 1240 ± | 3240 ± | 100 ± | 24 ± | 4.75 ± |
| | | | | 1.03 | 70.4 | 370 | 903 | 41.5 | 0 | NA |
| | 45 | Male | 120 | 2.00 ± | 313 ± | 2160 ± | 4210 ± | 96.5 ± | 24 ± | 5.7 ± |
| | | | | 5.72 | 76.6 | 620 | 983 | 45.3 | 0 | 1.95 |
| | | Female | 120 | 10.0 ± | 304 ± | 2230 ± | 4350 ± | 73.2 ± | 24 ± | 5.16 ± |
| | | | | 7.34 | 102 | 642 | 1070 | 27.4 | 0 | 0.817 |
| | 90 | Male | 120 | 14.0 ± | 250 ± | 1620 ± | 3480 ± | 76.5 ± | 24 ± | 5.15 ± |
| | | | | 4.32 | 35.6 | 286 | 516 | 15.1 | 0 | 0.626 |
| | | Female | 120 | 16.0 ± | 373 ± | 2180 ± | 4920 ± | 137 ± | 24 ± | 5.65 ± |
| | | | | 6.84 | 161 | 1030 | 1410 | 50.8 | 0 | 0.936 |
| Test | 1 | Male | 105 | 13.0 ± | 205 ± | 962 ± | 2920 ± | 141 ± | 24 ± | 12.55 ± |
| | | | | 4.69 | 67.4 | 418 | 874 | 52.4 | 0 | NA |
| | | Female | 105 | 18.0 ± | 220 ± | 758 ± | 2930 ± | 144 ± | 24 ± | 5.18 ± |
| | | | | 3.01 | 37.2 | 95.8 | 455 | 44 | 0 | NA |
| | 45 | Male | 105 | 16.0 ± | 244 ± | 1670 ± | 3840 ± | 132 ± | 24 ± | 5.78 ± |
| | | | | 2.45 | 86.1 | 223 | 713 | 49 | 0 | NA |
| | | Female | 105 | 13.0 ± | 233 ± | 1780 ± | 3810 ± | 115 ± | 24 ± | 10.3 ± |
| | | | | 8.43 | 55.6 | 655 | 947 | 31.5 | 0 | 4.22 |
| | 90 | Male | 105 | 15.0 ± | 210 ± | 1470 ± | 3320 ± | 99.4 ± | 24 ± | 8.87 ± |
| | | | | 4.08 | 28 | 285 | 490 | 38.6 | 0 | 0.294 |
| | | Female | 105 | 16.0 ± | 266 ± | 1280 ± | 3640 ± | 130 ± | 24 ± | NC |
| | | | | 2.07 | 70.5 | 173 | 389 | 15.6 | 0 | |
| | 1 | Male | 275 | 12.0 ± | 400 ± | 1780 ± | 5400 ± | 226 ± | 24 ± | 11.81 ± |
| | | | | 3.2 | 79.7 | 145 | 1120 | 103 | 0 | 8.66 |
| | | Female | 275 | 15.0 ± | 414 ± | 1710 ± | 5720 ± | 257 ± | 24 ± | 11.0 ± |
| | | | | 3.67 | 98.6 | 373 | 1260 | 118 | 0 | NA |
| | 45 | Male | 275 | 15.0 ± | 485 ± | 3480 ± | 7630 ± | 219 ± | 24 ± | 5.92 ± |
| | | | | H | = 80 | = 936 | + 1530 | = 106 | ± 0 | = 1.4 |
| | | | | 2.73 | | | | | | |
| | | Female | 275 | 15.0 ± | 566 ± | 4170 ± | 9170 ± | 334 ± | 24 ± | 5.80 ± |
| | | | | 7.62 | 90.8 | 1510 | 2690 | 213 | 0 | NA |
| | 90 | Male | 275 | 16.0 ± | 593 ± | 3060 ± | 8520 ± | 268 ± | 24 ± | 5.90 ± |
| | | | | 1.63 | 127 | 462 | 1780 | 104 | 0 | NA |
| | | Female | 275 | 15.0 ± | 626 ± | 3470 ± | 9630 ± | 324 ± | 24 ± | 5.19 ± |
| | | | | 2.34 | 154 | 1470 | 2930 | 127 | 0 | NA |

^{a} Tₘₐₓ, and Tₗₐₛₜ presented as median;
Data presented as mean ± SD,
n = 6 animals/ gender/dose level at 105, 275 and 120 mg/dog/day dose level;
NA—not applicable;
NC: Not Calculated due to non-linear elimination Phase where r² < 0.8

### CONCLUSIONS

A 90 day repeat dose administration of Test product to Beagle dogs at a dose of up to 275 mg/dog/day did not result in any adverse changes in any of the parameters evaluated in this study. Dogs on Reference product dose of 120 mg/dog/day showed no adverse changes in any of the parameters evaluated in this study. Based on the above findings, the No Observed Adverse Effect Level (NOAEL) of Test tablets is considered to be 275 mg/day/dog, which is equivalent to approximately 27.5 mg/kg/day.

1-45. (canceled)

46. A method of treating at least one symptom of neurogenic orthostatic hypotension, the method comprising orally administering to a person in need thereof an extended release composition comprising an immediate release component and an extended release component, wherein the immediate release component comprises an immediate release drug layer comprising from about 10 mg to about 100 mg of pyridostigmine or a pharmaceutically acceptable salt thereof;
wherein the extended release component comprises a core and a permeable elastic membrane comprising an orifice and surrounding the core;
wherein the core comprises from about 50 mg to about 200 mg of pyridostigmine or a pharmaceutically acceptable salt thereof,
wherein the permeable elastic membrane comprises a copolymer based on ethyl acrylate, methyl methacrylate, and trimethylammonioethyl methacrylate chloride, and
wherein the copolymer is present in an amount of from about 70 wt % to about 95 wt %, based on the total weight of the membrane.

47. The method of claim 46, wherein the at least one symptom is retinal hypoperfusion, muscle hypoperfusion, lung hypoperfusion, cerebral hypoperfusion, myocardial hypoperfusion, nonspecific symptoms, or a combination thereof.

48. The method of claim 47, wherein the retinal hypoperfusion is impaired vision.

49. The method of claim 47, wherein the muscle hypoperfusion is neck pain, shoulder pain, or a combination thereof.

50. The method of claim 47, wherein the lung hypoperfusion is orthostatic dyspnea.

51. The method of claim 47, wherein the cerebral hypoperfusion is dizziness, light headedness, pre-syncope, syncope, difficulty concentrating, headache, cognition, or a combination thereof.

52. The method of claim 47, wherein the myocardial hypoperfusion is angina.

53. The method of claim 47, wherein the nonspecific symptoms are generalized weakness, falls, leg buckling, lethargy, fatigue, nausea, or a combination thereof.

54. A method of treating neurogenic orthostatic hypotension, the method comprising orally administering to a person in need thereof an extended release composition comprising an immediate release component and an extended release component,
wherein the immediate release component comprises an immediate release drug layer comprising from about 10 mg to about 100 mg of pyridostigmine or a pharmaceutically acceptable salt thereof;
wherein the extended release component comprises a core and a permeable elastic membrane comprising an orifice and surrounding the core;
wherein the core comprises from about 50 mg to about 200 mg of pyridostigmine or a pharmaceutically acceptable salt thereof,
wherein the permeable elastic membrane comprises a copolymer based on ethyl acrylate, methyl methacrylate, and trimethylammonioethyl methacrylate chloride, and
wherein the copolymer is present in an amount of from about 70 wt % to about 95 wt %, based on the total weight of the membrane.

55. A method for treating neurogenic orthostatic hypotension in a person that did not respond to other treatments, the method comprising orally administering to a person in need thereof an extended release composition comprising an immediate release component and an extended release component,
wherein the immediate release component comprises an immediate release drug layer comprising from about 10 mg to about 100 mg of pyridostigmine or a pharmaceutically acceptable salt thereof;
wherein the extended release component comprises a core and a permeable elastic membrane comprising an orifice and surrounding the core;
wherein the core comprises from about 50 mg to about 200 mg of pyridostigmine or a pharmaceutically acceptable salt thereof,
wherein the permeable elastic membrane comprises a copolymer based on ethyl acrylate, methyl methacrylate, and trimethylammonioethyl methacrylate chloride, and
wherein the copolymer is present in an amount of from about 70 wt % to about 95 wt %, based on the total weight of the membrane.

56. A method for treating neurogenic orthostatic hypotension in persons that discontinued other treatments because of supine hypertension, the method comprising orally administering to a person in need thereof an extended release composition comprising an immediate release component and an extended release component,
wherein the immediate release component comprises an immediate release drug layer comprising from about 10 mg to about 100 mg of pyridostigmine or a pharmaceutically acceptable salt thereof;
wherein the extended release component comprises a core and a permeable elastic membrane comprising an orifice and surrounding the core;
wherein the core comprises from about 50 mg to about 200 mg of pyridostigmine or a pharmaceutically acceptable salt thereof,
wherein the permeable elastic membrane comprises a copolymer based on ethyl acrylate, methyl methacrylate, and trimethylammonioethyl methacrylate chloride, and
wherein the copolymer is present in an amount of from about 70 wt % to about 95 wt %, based on the total weight of the membrane.

57. A method for treating at least one symptom of neurogenic orthostatic hypotension caused by primary autonomic failure beta-hydroxylase deficiency, diabetic autonomic neuropathy, and/or non-diabetic autonomic neuropathy, the method comprising orally administering to a person in need thereof an extended release composition comprising an immediate release component and an extended release component,
wherein the immediate release component comprises an immediate release drug layer comprising from about 10 mg to about 100 mg of pyridostigmine or a pharmaceutically acceptable salt thereof;
wherein the extended release component comprises a core and a permeable elastic membrane comprising an orifice and surrounding the core;
wherein the core comprises from about 50 mg to about 200 mg of pyridostigmine or a pharmaceutically acceptable salt thereof,
wherein the permeable elastic membrane comprises a copolymer based on ethyl acrylate, methyl methacrylate, and trimethylammonioethyl methacrylate chloride, and
wherein the copolymer is present in an amount of from about 70 wt % to about 95 wt %, based on the total weight of the membrane.

58. The method of claim 57, wherein the primary autonomic failure comprises autonomic failure associated with Parkinson's disease (PD), autonomic failure associated with multiple system atrophy, or pure autonomic failure.

59. The method of claim 57, wherein the at least one symptom comprises orthostatic dizziness, lightheadedness, feeling like you might black out, cognitive slowing, sleepiness, presyncope, and syncope, increased risk of falls, cognitive impairment, or exercise intolerance.

60. A method for treating neurogenic orthostatic hypotension without worsening supine hypertension, the method comprising orally administering to a person in need thereof an extended release composition comprising an immediate release component and an extended release component,
wherein the immediate release component comprises an immediate release drug layer comprising from about 10 mg to about 100 mg of pyridostigmine or a pharmaceutically acceptable salt thereof;
wherein the extended release component comprises a core and a permeable elastic membrane comprising an orifice and surrounding the core;
wherein the core comprises from about 50 mg to about 200 mg of pyridostigmine or a pharmaceutically acceptable salt thereof,
wherein the permeable elastic membrane comprises a copolymer based on ethyl acrylate, methyl methacrylate, and trimethylammonioethyl methacrylate chloride, and
wherein the copolymer is present in an amount of from about 70 wt % to about 95 wt %, based on the total weight of the membrane.

61. The method of claim 60, wherein the core further comprises an acid, a gas generating agent, a filler, a wicking agent, a swellable water-soluble hydrophilic polymer, or combinations thereof.

62. The method of claim 61, wherein the acid is an organic acid selected from the group consisting of succinic acid, citric acid, acetic acid, malic acid, fumaric acid, stearic acid, tartaric acid, boric acid, benzoic acid, and a combination thereof.

63. The method of claim 61, wherein the gas generating agent is selected from the group consisting of sodium bicarbonate, sodium carbonate, magnesium carbonate, and calcium carbonate.

64. The method of claim 61, wherein the wicking agent is crospovidone.

65. The method of claim 61, wherein the filler is selected from the group consisting of lactose monohydrate, anhydrous lactose, directly compressible starches, hydrolyzed starches, pregelatinized starch, microcrystalline cellulose, silicified microcrystalline cellulose, carboxymethylcellulose and other cellulose polymers, sucrose and sucrose-based materials, dextrose, dibasic calcium phosphate anhydrous, dibasic calcium phosphate dihydrate, tricalcium phosphate, calcium sulfate dihydrate, and other alkaline inorganic salts, sugar alcohols such as mannitol, sorbitol, and xylitol, confectioner's sugar, and a combination thereof.

66. The method of claim 65, wherein the swellable water-soluble hydrophilic polymer is hydroxypropyl methylcellulose.

67. The method of claim 66, wherein the hydroxypropyl methylcellulose is a mixture low viscosity hydroxypropyl methylcellulose and a high viscosity hydroxypropyl methylcellulose.

68. The method of claim 67, wherein the low viscosity hydroxypropyl methylcellulose has a viscosity of from about 50 mPa·s to about 2,400 mPa·s, and a weight average molecular weight of from about 150,000 Da to about 300,000 Da; and wherein the high viscosity hydroxypropyl methylcellulose has a viscosity of from about 2,500 mPa·s to about 300,000 mPa·s, and a weight average molecular weight of from about 350,000 Da and about 1,500,000 Da.

69. The method of claim 60, wherein the treatment comprises an increase in diastolic blood pressure by at least about 5 mmHg post standing and an increase in systolic blood pressure by at least about 10 mmHg post standing.
